(19) United States
(12) Patent Application Publication
(10) Pub. No.: US 2021/0214731 A1
(43) Pub. Date: Jul. 15, 2021

(54) METHODS FOR TREATING CANCER

(71) Applicant: President and Fellows of Harvard College, Cambrigde, WA (US)

(72) Inventors: David T. Scadden, Weston, MA (US); Cheuk-Him Man, Kowloon (HK)

(21) Appl. No.: 17/016,209

(22) Filed: Sep. 9, 2020

Related U.S. Application Data
(60) Provisional application No. 62/926,301, filed on Oct. 25, 2019.

(51) Int. Cl. C12N 15/113; A61P 35/02; A61K 31/192; A61K 31/551

(52) U.S. Cl. CPC C12N 15/1138; A61P 35/02; C12N 2310/531; A61K 31/551; A61K 31/192

(57) ABSTRACT

Disclosed are methods for treating cancers (e.g., AML) having increased intracellular pH, including AML overexpressing MCT4. Also disclosed are methods of modulating cell growth by modulating intracellular pH.

### RELATED APPLICATION(S)

This application claims the benefit of U.S. Provisional Application No. 62/926,301, filed on Oct. 25, 2019, the entire teachings of which are incorporated herein by reference.

### GOVERNMENT SUPPORT

This invention was made with government support under CA193461 and CA194596 awarded by the National Institutes of Health. The government has certain rights in the invention.

### BACKGROUND OF THE INVENTION

Partitioning ions across membranes is a fundamental property of cellular life. Electrical charge gradients accompanying ion partitioning are a mechanism for storing energy and therefore are central to kinetic events in both prokaryotic and eukaryotic cells. Ion shifts serve to regulate cell programs such as apoptosis, ligand-receptor-based activation, migration and myofibril contraction. In plants, H+ ions are mediators of cell growth, though not proliferation. Auxins activate proton pumps that lower pH in the cell wall activating the proteins, expansins, that allow for relaxing cell wall stiffness and cell growth: the ‘acid growth theory’ of Cleland and colleagues (Rayle and Cleland, 1992). However, there is no clear corollary of acid enabling growth in mammalian cells.

Ions rarely travel alone and their movement in cells is coupled to other charged entities such as amino acids, proteins, drugs and products of carbon metabolism. Solute carrier proteins (SLC) are a ˜400-member family of integral membrane proteins many of whom transport ions. Among these, the monocarboxylate transporters (MCT) co-transport protons and monocarboxylates such as lactate and pyruvate (Adijanto and Philp, 2012). MCT1 (SLC16A1) and MCT4 (SLC16A3) are the major co-transporters for lactate uptake and efflux respectively. MCT4 is highly expressed and essential in glycolytic cells and its activity is driven by lactate gradients. As such, it is a means of limiting lactate intracellular accumulation with a secondary consequence of proton shifting extracellularly. It can therefore be viewed as a transporter whose activity is linked to a glucose replete environment: a cellular context of nutrient availability conducive to growth.

### SUMMARY OF THE INVENTION

The inventors have surprisingly discovered that increasing intracellular pH (pHi) increases the growth of a number of different cell types including AML via increasing glycolysis and PPP activity. In AML, the inventors have found that this increased growth is mediated via over-expression of MCT4. Inhibiting MCT4 preferentially inhibited the growth and viability of leukemic cells and surprisingly eliminated leukemic initiating cells (LICs) without affecting HSPC growth. Modulating pHi via proton exporters such as MCT4 or NHE1 can provide new therapeutic modalities, especially for cancer.

Some aspects of the present invention are directed to a method of treating leukemia in a subject in need thereof, comprising administering to the subject a therapeutically effective amount of an agent that inhibits the activity or expression of a proton exporter.

In some embodiments, the proton exporter is Monocarboxylate Transporter 4 (MCT4). In some embodiments, the agent does not inhibit Monocarboxylate Transporter 1 (MCT1) activity or expression. In some embodiments, the proton exporter is NHE1. In some embodiments, the agent inhibits the growth, viability or clonogenic ability of leukemia initiating cells (LICs). In some embodiments, the agent preferentially inhibits the growth, viability or clonogenic ability of leukemia cells in the bone marrow. In some embodiments, the leukemia exhibits increased intracellular pH (pHi) as compared to non-leukemic blood cells. In some embodiments, the leukemic cells exhibit increased transcriptional activation marks in the MCT4 promoter region as compared to non-leukemic blood cells. In some embodiments, the leukemic cells exhibit increased MCT4 expression or activity. In some embodiments, the agent does not inhibit the growth, viability, or clonogenic ability of non-leukemic blood cells. In some embodiments, the agent comprises a protein, nucleic acid, or small molecule.

In some embodiments, the subject is administered a second anti-leukemic (anti-cancer) agent. In some embodiments, the second anti-leukemic agent is a glycolysis inhibitor, a histone deacetylase inhibitor, or a pentose phosphate pathway (PPP) inhibitor. In some embodiments, the subject is administered a second agent selected from a pro-apototic agent (e.g., venetoclax), an agent that enhances non-caspase dependent cell death (e.g., GPX4 inhibitor), an immunotherapeutic agent (e.g., CD47 inhibitor, checkpoint inhibitor), a antibody drug conjugate, a Bi-specific T-cell engager (BiTE), dual ipilimumab and nivolumab therapy (DART), or an immunologic cell therapy (e.g., NK-CAR, CAR-T).

In some embodiments, administration of the agent substantially or completely eliminates LICs from the subject.

Some aspects of the present invention are directed to a method of inhibiting the growth, viability, or clonogenic ability of a cancer cell, comprising contacting the cancer cell with an agent that decreases the intracellular pH (pHi) of the cancer cell.

In some embodiments, the cancer cell exhibits increased intracellular pH (pHi) as compared to a non-cancer cell. In some embodiments, the cancer is glycolysis dependent. In some embodiments, the cancer is not OXPHOS-dependent. In some embodiments, the cancer cell comprises an oncogenic protein having increased activity at increased pHi. In some embodiments, the cancer cell exhibits increased activity or expression of a proton exporter as compared to a non-cancer cell. In some embodiments, the proton exporter is Monocarboxylate Transporter 4 (MCT4). In some embodiments, the proton exporter is NHE1. In some embodiments, the agent inhibits the activity or expression of the proton exporter in the cancer cell.

In some embodiments, the agent does not inhibit MCT4 activity or expression. In some embodiments, the agent does not inhibit MCT1 activity or expression. In some embodiments, the agent preferentially inhibits the growth, viability, or clonogenic ability of a cancer cell in a low oxygen (e.g., hypoxic) environment. In some embodiments, the agent does not inhibit the growth or viability of non-cancerous cells. In some embodiments, the agent preferentially inhibits the growth or viability of cancerous cells as compared to non-cancerous cells. In some embodiments, the cancer is leukemia.

In some embodiments, the agent is administered to a subject having cancer. In some embodiments, the subject is administered a second anti-cancer agent. In some embodiments, the second anti-cancer agent is a glycolysis inhibitor, a histone deacetylase inhibitor, or a pentose phosphate pathway (PPP) inhibitor. In some embodiments, the subject is administered a second agent selected from a pro-apototic agent (e.g., venetoclax), an agent that enhances non-caspase dependent cell death (e.g., GPX4 inhibitor), an immunotherapeutic agent (e.g., CD47 inhibitor, checkpoint inhibitor), a antibody drug conjugate, a Bi-specific T-cell engager (BiTE), dual ipilimumab and nivolumab therapy (DART), or an immunologic cell therapy (e.g., NK-CAR, CAR-T).

Some aspects of the present invention are directed to a method of preventing, delaying, reducing the likelihood of relapse of, or reducing the likelihood of leukemia in a subject in need thereof, comprising administering to the patient a therapeutically effective amount of a proton exporter (e.g., MCT4, NHE1) inhibitor.

In some embodiments, the subject has one or more risk factors associated with the development of leukemia. In some embodiments, the one or more risk factors include advanced age or the presence of a gene mutation. In some embodiments, the one or more risk factors is previous leukemia in the subject.

Some aspects of the present invention are directed to a method of determining if a cancer is responsive to MCT4 inhibition therapy, comprising determining if the level of transcriptional activation marks on the MCT4 promoter of the cancer cell is elevated as compared to a control non-cancerous cell. In some embodiments, the cancer is leukemia. In some embodiments, the level of transcriptional activation marks on the MCT4 promoter is determined by ChIP-PCR. In some embodiments, if the level of transcriptional activation marks on the MCT4 promoter of the cancer cell is elevated, then the cancer cell is contacted with an agent as described herein.

Some aspects of the present invention are directed toward a method of determining if a cancer is responsive to MCT4 inhibition therapy, comprising determining if the level of MCT4 expression in the cancer cell is elevated as compared to a control non-cancerous cell. In some embodiments, the cancer is leukemia. In some embodiments, if the level of MCT4 expression or activity is elevated, then the cancer cell is contacted with an agent as described herein.

Some aspects of the present invention are directed toward a method of increasing the growth or proliferation of a cell comprising contacting the cell with an agent that increases the expression or activity of Monocarboxylate Transporter 4 (MCT4). Some aspects of the present invention are directed toward a method of increasing the growth or proliferation of a cell comprising contacting the cell with an agent that increases the expression or activity of NHE1. In some embodiments, the cell is a hematopoietic stem or progenitor cell, myeloid hematopoietic cell, pre-osteoblast, primary tracheal epithelial cell, or primary bronchial epithelial cell.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1J show alkaline pHi and MCT4 upregulation are common features in AML. (FIG. 1A) Ex vivo pHi of mouse AML and normal blood cells examined using SNARF-1 by FACS (n=3-4). Ex vivo pHi of (FIG. 1B) human leukemic cell lines (n=3), (FIG. 1C) primary human AML (n=12) and primary AML LIC (n=7) with CB HSC1 (n=3), CB HSC2 (n=3), CB (n=7) and BMMC (n=7) examined using SNARF-1 by FACS. (FIG. 1D) In vivo pHi of 100 normal blood cells and 100 MLL-AF9 AML (from 3 mice) imaged by multiphoton fluorescent microscope were determined based on the calibrated standard cell shown Fig. S1D. (FIG. 1E) Q-PCR analysis of transcriptional expression of various pH regulators in MLL-AF9, HoxA9-Meis1, PML-RARα mouse AML and mouse WBM (n=3). (FIG. 1F) Q-PCR analysis of transcriptional expression of MCT4 in mouse LT-HSC (n=6), ST-HSC (n=6), MLL-AF9 AML LIC (n=3) and HoxA9-Meis1 AML LIC (n=3). (FIG. 1G) Q-PCR analysis of transcriptional expression of MCT4 in human CB HSC1 (n=3), CB HSC2 (n=3), CB MPP (n=3), CB progenitor (n=3), primary AML LIC (n=6) and primary bulk AML (n=6). (FIG. 1H) Western blot of MCT4 protein in primary human AML (n=16) and CB (n=3). (FIG. 1I) Mann-Whitney U test analysis of the MCT4 protein expression in AML and CB from FIG. 1F. (FIG. 1J) Kaplan Meier survival analysis of AML patients with either high or low MCT4 expression (TCGA-LAML).

FIGS. 2A-2I also show alkaline pHi and MCT4 upregulation are common features in AML. (FIG. 2A) FACS analysis of the mCherry-SEpHluorin expressing AML calibrated at pH 7.2 and 7.6 buffers in vitro. The intensity of mCherry was consistent at both pH, while SEpHluorin decreased at more acidic pH. (FIG. 2B) In vitro pHi of MLL-AF9 AML and normal LKS as determined by FACS using mCherry-SEpHluorin pH reporter (n=3). (FIG. 2C) mCherry-SEpHluorin expressing MLL-AF9 was calibrated at different pH conditions and imaged by in vitro multiphoton fluorescent microscope. At pH7.0, the cell was reddish-yellow color. At pH7.5, the color was yellowish-green. At pH8.0, the color was green. (FIG. 2D) Representative images examined by in vivo multiphoton fluorescent microscope showing that mouse MLL-AF9 AML was more greenish color, while normal blood cells were more reddish in mouse calvarial BM cavity. (FIG. 2E) Re-analysis of GSE20377 showing the relative mRNA expression of various pH regulators in MLL-AF9 AML and normal HSPCs. (FIG. 2F) Analysis of MCT4 protein in MLL-AF9 AML and normal GMP examined by FACS (n=3). (FIG. 2G) Relative mRNA expression of MCT4 in human leukemic cell lines and CB by Q-PCR (n=3). (FIG. 2H) Re-analysis of GSE9476 showing the mRNA expression of MCT4 in primary AML samples (n=26) and CB (n=18). (FIG. 2I) Relative mRNA expression of MCT4 in normal mouse HSPC and various hematologic malignancies by Q-PCR (n=3).

FIGS. 3A-3Q show MCT4 inhibition suppressed AML growth. (FIG. 3A) Western blot of MCT4 protein in MLL-AF9 mouse AML with indicated MCT4-knockout by gRNAs in vitro. (FIG. 3B) pHi and (FIG. 3C) intracellular lactate in mouse AML with MCT4-KO in vitro (n=4). (FIG. 3D) In vitro growth of MLL-AF9 AML upon MCT4-KO (n=4). (FIG. 3E) Apoptosis analysis and (FIG. 3F) BrdU incorporation assay in MCT4-KO AML in vitro (n=4). (FIG. 3G) Western blot of cell cycle related proteins in MCT4-KO AML. (FIG. 3H) In vivo pHi of MLL-AF9 AML with scrambled shRNA control (n=70) and MCT4-KD (n=70) (from 3 mice) imaged by multiphoton fluorescent microscope were determined based on the calibrated standard cell shown in FIG. 2D. (FIG. 3I) Kaplan Meier survival analysis of mice transplanted with MLL-AF9 AML upon in vivo induced MCT4 (n=8 and 9) or scrambled shRNA (n=8). Red area indicates doxycycline induction. (FIG. 3J) FASC plots showing the proportion of host leukocytes (CD45.2) and AML (CD45.1) in recipient BM at 60-day post doxycycline withdrawal and 24 weeks post-secondary transplantation. (FIG. 3K) Serial in vitro colony forming assay of mouse cKit⁺ MLL-AF9 AML with MCT4-KO (n=3, triplicate wells for each experiment). (FIG. 3L) In vivo engraftment of mouse HoxA9-Meis1 and PML-RARα AML with MCT4-KD (n=7) or scrambled shRNA control (n=7). (FIG. 3M) FACS analysis of MCT4 protein expression in primary human AML samples (n=6) with MCT4-KD by shRNA and scrambled shRNA control. (FIG. 3N) pHi and (FIG. 3O) intracellular lactate in primary human AML with MCT4-KD (n=4) ex vivo. (FIG. 3P) In vivo engraftment of primary human AML with MCT4-KD by shRNA or scrambled shRNA in NSG (9 individual AML patient samples, 1-2 mice per each sample). (FIG. 3Q) Serial in vitro colony forming assay of human CD34⁺CD38⁻ primary AML with MCT4-KO (5 individual AML patient samples, triplicate wells for each experiment).

FIGS. 4A-4AC further show MCT4 inhibition suppressed AML growth. (FIG. 4A) HPLC analysis of extracellular lactate from the media cultured with MCT4-KO AML in vitro after 2 hours incubation (n=3). (FIG. 4B) In vitro colony forming assay of mouse MLL-AF9 AML with MCT4-KO (n=3, triplicate wells for each experiment). (FIG. 4C) In vitro pHi change, (FIG. 4D) in vitro growth change and (FIG. 4E) in vitro change of BrdU incorporation of MLL-AF9 AML with MCT4 normalized to non-targeting gRNA control (n=4). (FIG. 4F) Western blot of HIF-1α and MCT4 protein of MLL-AF9 AML in 20% and 2% O₂ in vitro. (FIG. 4G) In vitro glucose uptake and (FIG. 4H) extracellular glucose of AML cultured in 2% O₂ (n=3). (FIG. 4I) MFI of CellTrace™ in MLL-AF9 AML upon MCT4-KO after 2 days in vitro culture. (FIG. 4J) Distribution of cell populations in G1, S and G2/M phase in cell cycle upon MCT4-KO in MLL-AF9 AML in vitro (n=4). (FIG. 4K) Representative images of Wright-Giemsa staining showing the morphology of AML at day-6 post Cas9 activation in vitro. (FIG. 4L) Correlation between extracellular pH and intracellular pH in MLL-AF9 mouse AML (n=3). (FIG. 4M) Level of intracellular lactate in AML cultured at pH 7.3 media supplemented with 2 mM and 10 mM lactate in vitro (n=3). (FIG. 4N) Fractional enrichment of intracellular lactate and pyruvate in MLL-AF9 AML cultured at pH 7.3 media supplemented with 2 mM and 10 mM ¹³C-labelled lactate in vitro (n=3). (FIG. 4O) Growth and (FIG. 4P) BrdU incorporation of MLL-AF9 AML cultured in media with different pH in vitro (n=3). (FIG. 4Q) Western blot analysis of protein expression of MCT4 in mouse MLL-AF9 AML with MCT4-knockdown and MCT4-overexpression in vitro (n=3). (FIG. 4R) In vitro growth of mouse MLL-AF9 AML with MCT4-knockdown and MCT4-overexpression (n=3). (FIG. 4S) Representative images of multiphoton fluorescent microscope showing in vivo pHi from pH reporter expressing AML upon 2-day post in vivo induced MCT4-KD by shRNA. (FIG. 4T) Correlation between MCT4 mRNA expression and the growth inhibition by MCT4-KD (n=10). (FIG. 4U) Western blot of MCT4 protein in THP-1 with MCT4-KD in vitro. (FIG. 4V) pHi and (FIG. 4W) intracellular lactate in THP-1 with MCT4-KD in vitro (n=3). (FIG. 4X) Western blot of MCT4 protein in THP-1 with MCT4-overexpression and -knockdown in vitro. (FIG. 4Y) In vitro growth of THP-1 with MCT4-overexpression and -knockdown. (FIG. 4Z) THP-1 engraftment with scrambled or MCT4 shRNA in NSG mice (n=4). (FIG. 4AA) MOLM-14 engraftment with scrambled or MCT4 shRNA in NSG mice (n=4). GSEA showing differential expressed genes of (FIG. 4AB) Hypoxia and (FIG. 4AC) E2F Targets gene sets in MCT4-KO MLL-AF9 AML.

FIGS. 5A-5M show upregulation of MCT4 is essential in AML-adapted glucose metabolism. (FIG. 5A) An overview of glucose metabolism. (FIG. 5B) Extracellular acidification rate (ECAR) and (FIG. 5C) oxygen consumption rate (OCR) in MLL-AF9 mouse AML upon MCT4-KO in vitro (n=5). (FIG. 5D) In vitro (MCT4-KO by CRISPR-Cas9, n=3) and (FIG. 5E) in vivo glucose uptake (MCT4-KD by shRNA, n=6-8) in MLL-AF9 AML. (FIG. 5F) In vitro intracellular metabolite profiling by LC-MS showing the relative levels of glycolytic metabolites and ATP:ADP ratio in MCT4-KO AML in 2% O₂ (n=3-5). (FIG. 5G) In vitro enzymatic activities of HK1, PFK1, GAPDH, PGK, PGM and PKM2 at different pH (n=3). Red area indicates the range of pH change from leukemic (pH7.6) to normal (pH7.3). Paired t-test comparison between pH7.3 with various pH levels. (FIG. 5H) In vitro intracellular metabolite profiling by LC-MS showing the relative levels of PPP metabolites, ratio of NADP⁺:NADPH and nucleotides of MCT4-KO AML in 2% O₂ (n=3-5). (FIG. 5I) In vitro cell growth of MCT4-KO AML supplemented with 100 mM nucleosides [Nu], 10 mM ribose [R], 0.5 mM pyruvate [P] or combination of ribose and pyruvate [R+P] (n=3). (FIG. 5J) In vitro glucose uptake of human primary AML with scrambled shRNA (control) or MCT4-knockdown (8 individual AML patient samples). (FIG. 5K) In vitro cell growth of MCT4-knockdown in primary AML samples supplemented with combination of 10 mM ribose and 0.5 mM pyruvate (5 individual AML patient samples). (FIG. 5L) In vitro enzymatic activities of G6PDH and PGD at different pH (n=3). Red area indicates the range of pH change from leukemic (pH7.6) to normal pHi (pH7.3). Paired t-test comparison between pH7.3 with various pH levels. (FIG. 5M) Percentage enrichment of ¹³C from ¹³C-U6-glucose in glycolytic, PPP metabolites and amino acids in 2% oxygen for 15- and 30 minutes in in vitro culture (n=3-4).

FIGS. 6A-6V further show upregulation of MCT4 is essential in AML-adapted glucose metabolism. (FIG. 6A) ECAR and OCR of mouse MLL-AF9 AML treated with LDHA inhibitor (FX11), hexokinase inhibitor (2DG) and pan-MCT inhibitor (aCHC) in vitro (n=3). (FIG. 6B) Cellular ROS and (FIG. 6C) mitochondrial ROS analysis in MLL-AF9 AML with MCT4 or LDHA-KO in vitro (n=4). (FIG. 6D) In vitro growth of MLL-AF9 with MCT4-KO treated with 2DG and 6AN (n=3). (FIG. 6E) Extracellular glucose cultured with MLL-AF9 AML upon MCT4-KO in vitro (n=3). (FIG. 6F) In vitro intracellular metabolite profiling by LC-MS showing the relative levels of glycolytic metabolites and ATP:ADP ratio in MCT4-KO AML in 20% O₂ (n=3-5). (FIG. 6G) In vitro cell growth of AML upon MCT4-KO adding 0.5 mM pyruvate [P] or 2 mM lactate [L] in 2% O₂ (n=3). (FIG. 6H) In vitro glucose uptake of MLL-AF9 AML cultured in media with different pH (n=3). (FIG. 6I) In vitro intracellular metabolite profiling by LC-MS showing the relative levels of glycolytic and PPP metabolites and ATP:ADP, NADP⁺:NADPH ratio in MLL-AF9 AML cultured in media with different pH (n=3-4). (FIG. 6J) In vitro glucose uptake of MLL-AF9 AML cultured in media with different lactate concentrations (n=3). (FIG. 6K) In vitro intracellular metabolite profiling by GC-MS showing the relative levels of glycolytic and TCA metabolites in MLL-AF9 AML cultured in media with different lactate concentrations (n=3-4). (FIG. 6L) Intracellular lactate analyzed by GC-MS in MLL-AF9 with MCT4-KO or cultured in 2 mM lactate (n=3-4). (FIG. 6M) Western blot of select enzymes upon MCT4-KO AML in 20% and 2% O₂ in vitro. In vitro enzymatic activity assay of (FIG. 6N) HK1 and (FIG. 6O) PKM2 in different conditions (pH7.3-7.6 in the presence of 2 mM lactate) (n=3). (FIG. 6P) The ratio of M+1/M+2 lactate in MLL-AF9 AML upon MCT4-KO in vitro. (FIG. 6Q) In vitro intracellular metabolite profiling by LC-MS showing the relative levels of PPP metabolites and NADP⁺:NADPH ratio in MCT4-KO AML in 20% O₂ (n=3-5). (FIG. 6R) In vitro glucose uptake of human AML cell lines, THP-1 and MOLM-14, with MCT4-KD (n=3). (FIG. 6S) In vitro intracellular metabolite profiling by LC-MS showing the relative levels metabolites in MCT4-KD THP1 (n=3). (FIG. 6T) In vitro enzymatic activity assay of G6PDH in different conditions (pH7.3-7.6 in the presence of 2 mM lactate) (n=3). (FIG. 6U) Western blot showing the ratio of HA:FLAG tagged protein after immunoprecipitation against FLAG tag and washed in different pH buffers. (FIG. 6V) An overview of the change of enzymatic activity upon MCT4-KO and the carbon flux on glucose metabolite and derived amino acids.

FIGS. 7A-7J show normal HSPCs are independent of MCT4 but depend on MCT1. (FIG. 7A) Normal CD45.1 LKS was infected with MCT4/MCT1 shRNA or scrambled shRNA and transplanted into primary recipient mice (n=6-7) with CD45.2 carrier whole bone marrow. Reconstituted CD45.1 and CD45.2 white blood cells (WBC), myeloid, B and T cells were examined every 4 weeks post transplantation until week 16 and the percentage of CD45.1 chimerism was evaluated. (FIG. 7B) At week-16 post-transplant, mouse BM was harvested. Different HSPCs in BM were examined by FACS. (FIG. 7C) WBM from primary transplant were injected into secondary recipient mice. WBC, myeloid, B and T cells were traced for 16 weeks (n=4). (FIG. 7D) HSPC in BM were harvested and examined at week-16. (FIG. 7E) Serial in vitro colony forming assay of human cord blood CD34⁺ cells with MCT4-KD (n=3, triplicate wells for each experiment). (FIG. 7F) In vitro intracellular metabolite profiling by LC-MS showing the relative levels of glycolytic, PPP, TCA metabolites and nucleotides of MCT4-KD cord blood CD34⁺ cells in 20% O₂ (n=3). (FIG. 7G) In vitro glucose uptake of cord blood CD34⁺ cells with MCT4-KD (n=4). (FIG. 7H) Intracellular pH analysis in cord blood CD34⁺ cells upon MCT1- or MCT4-KD (n=4). (FIG. 7I) Serial in vitro colony forming assay of human cord blood CD34⁺ cells with MCT1-KD (n=3, triplicate wells for each experiment). (FIG. 7J) In vitro glucose uptake in cord blood CD34⁺ cells with MCT1-KD (n=4).

FIGS. 8A-8Q also show normal HSPCs are independent of MCT4 but depend on MCT1. (FIG. 8A) Western blot of MCT4 protein in normal Lin⁻ BM cells with MCT4-KD in vitro. (FIG. 8B) In vitro growth of normal LKS with MCT4-KD (n=3). (FIG. 8C) In vitro colony forming assay of normal LKS with MCT4-KD (n=3, triplicate wells for each experiment). (FIG. 8D) Normal CD45.1 LKS was infected with MCT4 or scrambled shRNA and transplanted into lethally irradiated primary CD45.2 recipient mice (n=6-7) with CD45.2 carrier whole bone marrow. Number of reconstituted white blood cells (WBC), myeloid, B and T cells were examined every 4 weeks post transplantation until week 16. (FIG. 8E) At week-16 post-transplant, mouse BM was harvested. The number of different HSPCs in BM were examined by FACS. (FIG. 8F) WBM from primary transplant were injected into secondary recipient mice. Number of WBC, myeloid, B and T cells were traced for 16 weeks (n=4). (FIG. 8G) Number of HSPC in BM were harvested and examined at week-16. (FIG. 8H) Q-PCR analysis of MCT1 mRNA expression in normal LKS upon MCT1-KD by shRNA in vitro (n=3). (FIG. 8I) Western blot of MCT1 protein in normal Lin⁻ BM cells with MCT1-KD in vitro. (FIG. 8J) In vitro pHi and (FIG. 8K) in vitro BrdU incorporation assay of normal LKS with MCT1/MCT4-KD. (FIG. 8L) Normal CD45.1 LKS was infected with MCT1 or scrambled shRNA and transplanted into lethally irradiated primary CD45.2 recipient mice (n=3) with CD45.2 carrier whole bone marrow. Number of reconstituted white blood cells (WBC), myeloid, B and T cells were examined every 4 weeks post transplantation until week 16. (FIG. 8M) At week-16 post-transplant, mouse BM was harvested. The number of different HSPCs in BM were examined by FACS. (FIG. 8N) Growth of MLL-AF9 AML with the knockout of different pH regulators in vitro by CRISPR-Cas9 (Averaged growth from 3-4 individual gRNA sequences, 4 replicates of experiment). (FIG. 8O) In vitro glucose uptake of normal GMP with MCT1/MCT4-KD (n=3). (FIG. 8P) Glucose and lactate levels in media cultured with MCT1/MCT4-KD normal GMP (n=3). (FIG. 8Q) Western blot of MCT1 protein in MLL-AF9 AML upon MCT4-KO in 2% O₂ in vitro.

FIGS. 9A-9M show epigenetic regulation of MCT4 expression by histone modification. (FIG. 9A) ChIP-PCR analysis of the enrichment of histone activation marks, H3K27ac and H3K4me3 on MCT4 promoter in mouse AML (MLL-AF9, HoxA9-Meis1 and PML-RARα) and normal Lin⁻ BM cells (n=3). (FIG. 9B) ChIP-PCR analysis of the enrichment of H3K27ac and H3K4me3 on MCT4 promoter in human leukemic cell lines (n=3). (FIG. 9C) Correlation between H3K27ac enrichment on MCT4 promoter with MCT4 expression in human AML cell lines (n=8). (FIG. 9D) ChIP-PCR analysis of the enrichment of H3K27ac on MCT4 promoter in primary human AML blasts (n=5) and cord blood CD34⁺ cells (n=3). (FIG. 9E) Correlation between H3K27ac enrichment on MCT4 promoter with MCT4 expression in primary AML samples (n=5) and cord blood CD34⁺ cells (n=3). (FIG. 9F) ChIP-PCR analysis of the enrichment of MLL-AF9 and BRD4 on the promoters of HoxA9 and MCT4 in mouse MLL-AF9 AML (n=3). (FIG. 9G) [left] mRNA and [right] protein expression of MCT4 in MLL-AF9 AML upon JQ-1, EPZ-5676 and MI-2-2 treatment in vitro (n=3). (FIG. 9H) JQ-1 treatment on the growth of MLL-AF9 AML. ChIP-PCR analysis of the enrichment of (FIG. 9I) BRD4 and (FIG. 9J) H3K27ac on MCT4 promoter in MLL-AF9 AML upon JQ-1 treatment in vitro (n=3). Effect of JQ-1 (100 nM) treatment on (FIG. 9K) MCT4 expression, (FIG. 9L) enrichment of H3K27ac on MCT4 promoter and (FIG. 9M) growth of primary human AML samples (5 individual AML patient samples).

FIGS. 10A-10H also show epigenetic regulation of MCT4 expression by histone modification. (FIG. 10A) Q-PCR analysis of MCT4 mRNA expression at day-3 post FLT3-ITD or MLL-AF9 overexpression in normal LKS in vitro (n=3-6). (FIG. 10B) In vitro pHi of normal LKS with FLT3-ITD, MLL-AF9 or MLL-AF9/MCT4-KD at day-3 post infection (n=3-4). (FIG. 10C) Q-PCR analysis of MCT4 in MLL-AF9 with MLL-AF9-KD by shRNA in vitro (n=3). (FIG. 10D) Correlation between methylation level on MCT4 promoter and MCT4 mRNA expression (data from TCGA-LAML). (FIG. 10E) Correlation between H3K4me3 enrichment on MCT4 promoter and MCT4 expression in human AML cell lines (n=8). (FIG. 10F) Dual luciferase reporter assay showing the luciferase expression corresponding to MCT4 promoter activity upon MLL-AF9 overexpression in HEK293T cell line in vitro (n=3). (FIG. 10G) Q-PCR analysis of MCT4 mRNA expression in THP-1 and NOMO-1 treated with BRD4 inhibitor, JQ-1, in vitro (n=3). (FIG. 10H) In vitro growth of THP1 and NOMO-1 treated with JQ-1 (n=3).

FIGS. 11A-11S show MCT4 upregulation is sufficient to induce normal cell growth and is critical for leukemogenesis. (FIG. 11A) In vitro BrdU incorporation assay of MLL-AF9 induced GMP with scrambled or MCT4 shRNA (n=3). (FIG. 11B) In vitro glucose uptake analysis of MLL-AF9 induced GMP with scrambled or MCT4 shRNA (n=4). (FIG. 11C) In vitro intracellular metabolite profiling by LC-MS of MLL-AF9 induced GMP (n=3-4). (FIG. 11D) Schematic illustration of the experiment in FIG. 11E. MLL-AF9 with MCT4/scrambled shRNA were infected to LKS. Infected cells were selected by GFP and transplanted into mice. (FIG. 11E) Kaplan Meier survival analysis of mice transplanted with MLL-AF9 retrovirally transduced LKS with scrambled (n=5) or MCT4 shRNA (n=6). (FIG. 11F) pHi and (FIG. 11G) cellular growth in LKS with MCT4-OE in vitro (n=3). (FIG. 11H) In vitro BrdU incorporation assay of LKS with MCT4-OE (n=5). (FIG. 11I) At week-16 post-transplant, mice BM was harvested (n=4-6). Donor cell chimerism of HSPCs in BM were examined by FACS. CD45.1 LKS was infected with empty vector or MCT4 and transplanted into recipient mice with CD45.2 carrier cells (n=4-6). (FIG. 11J) Reconstituted WBC and (FIG. 11K) myeloid were examined every 4 weeks post transplantation until week 12 and the percentage of CD45.1 chimerism was evaluated. (FIG. 11L) ECAR and OCR of Lin⁻ BM cells with MCT4-OE in vitro (n=3). (FIG. 11M) In vitro glucose uptake in Lin⁻ BM cells with MCT4-OE (n=3). (FIG. 11N) In vitro intracellular metabolite profiling by GC/LC-MS showing the relative levels of metabolites in Lin⁻ BM cells with MCT4-OE (n=3). (FIG. 11O) pHi of human cord blood CD34⁺ cells with MCT4-OE in vitro (n=4). (FIG. 11P) In vitro colony forming assay of human cord blood CD34⁺ cells with MCT4-OE (n=4, triplicate wells for each experiment). (FIG. 11Q) In vitro glucose uptake in human cord blood CD34⁺ cells with MCT4-OE (n=4). (FIG. 11R) In vitro cellular growth of MC3T3 (n=5) and CD1 (n=3) upon MCT4 overexpression. (FIG. 11S) pHi analysis of MC3T3 (n=3) and CD1 (n=3) by SNARF-1 upon MCT4 overexpression.

FIGS. 12A-12C also show MCT4 upregulation is sufficient to induce normal cell growth and is critical for leukemogenesis. (FIG. 12A) Western blot of MCT4 protein in normal mouse Lin⁻ BM cells upon MCT4 overexpression. (FIG. 12B) Number of reconstituted WBC, myeloid, B and T cell from mice transplanted with CD45.1 MCT4-OE cell and CD45.2 carrier cells for 12 weeks post transplantation (n=3). (FIG. 12C) Number of LKS, CMP, GMP and MEP from the bone marrow of mice transplanted with CD45.1 MCT4-OE cell and CD45.2 carrier cells at 16-week post transplantation (n=4).

FIGS. 13A-13B shows inhibition of NHE1 in human AML reduces pH and glucose uptake. (FIG. 13A) Therapeutic inhibition of NHE1 in human AML cell lines resulted in reduction in intracellular pH. (FIG. 13B) Therapeutic inhibition of NHE1 in human AML cell lines resulted in reduction in glucose uptake.

FIG. 14 shows overexpressing NHE1 in cord blood CD34+ cell increase intracellular pH (n=4).

FIGS. 15A-15B show NHE1 increases cell growth. (FIG. 15A) Overexpressing NHE1 increased cord blood CD34+ growth in vitro (n=4). (FIG. 15B) Overexpressing NHE1 in cord blood increased myeloid cells in NSG mice at 4 weeks. (n=4).

FIG. 16 is an illustration of cellular pH regulators.

FIG. 17 is an illustration of biosynthesis and bioenegenesis in some AML cells not utilizing glycolysis.

### DETAILED DESCRIPTION OF THE INVENTION

### Some Definitions

As used herein, a “subject” means a human or animal “Subject” and “patient” may be used interchangeably herein. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. In some embodiments, the subject has cancer. In some embodiments, the subject has leukemia (e.g., AML).

As used herein, the term “treating” and “treatment” refers to administering to a subject an effective amount of an agent so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment.

As used herein, the term “therapeutically effective amount” means an amount of the agent which is effective to treat a disease (e.g., leukemia, cancer). Determination of a therapeutically effective amount is well within the capability of those skilled in the art. Generally, a therapeutically effective amount can vary with the subject's history, age, condition, sex, as well as the severity and type of the medical condition in the subject, and administration of other agents that treat the disease (e.g., leukemia, cancer).

As used herein, “administering” is not limited. In some embodiments, the agents described herein are administered, e.g., implanted, e.g., orally, systemically, sub- or trans-cutaneously, as an arterial stent, surgically, or via injection. In some examples, the agents described herein are administered by routes such as injection (e.g., subcutaneous, intravenous, intracutaneous, percutaneous, or intramuscular) or implantation.

In some embodiments, the agent is administered once every day to once every 10 years (e.g., once every day, once every week, once every two weeks, once every month, once every two months, once every 3 months, once every 4 months, once every 5 months, once every 6 months, once every year, once every 2 years, once every 3 years, once every 4 years, once every 5 years, once every 6 years, once every 7 years, once every 8 years, or once every 10 years). In other examples, the composition is administered once to 5 times (e.g., one time, twice, 3 times, 4 times, 5 times, or more as clinically necessary) in the subject's lifetime.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In some embodiments, the agent is selected from the group consisting of a nucleic acid, a small molecule, a polypeptide, and a peptide. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

“Small molecule” is defined as a molecule with a molecular weight that is less than 10 kD, typically less than 2 kD, and preferably less than 1 kD. Small molecules include, but are not limited to, inorganic molecules, organic molecules, organic molecules containing an inorganic component, molecules comprising a radioactive atom, synthetic molecules, peptide mimetics, and antibody mimetics. As a therapeutic, a small molecule may be more permeable to cells, less susceptible to degradation, and less apt to elicit an immune response than large molecules.

As used herein, the term “polypeptide” is used to designate a series of amino acid residues connected to the other by peptide bonds between the alpha-amino and carboxy groups of adjacent residues. The term “polypeptide” refers to a polymer of protein amino acids, including modified amino acids (e.g., phosphorylated, glycated, glycosylated, etc.) and amino acid analogs, regardless of its size or function. The term “peptide” is often used in reference to small polypeptides, but usage of this term in the art overlaps with “protein” or “polypeptide.” Exemplary polypeptides include gene products, naturally occurring proteins, homologs, orthologs, paralogs, fragments and other equivalents, as well as both naturally and non-naturally occurring variants, fragments, and analogs of the foregoing.

The term “nucleic acid” refers to polynucleotides such as deoxyribonucleic acid (DNA) and ribonucleic acid (RNA). The terms “nucleic acid” and “polynucleotide” are used interchangeably herein and should be understood to include double-stranded polynucleotides, single-stranded (such as sense or antisense) polynucleotides, and partially double-stranded polynucleotides. A nucleic acid often comprises standard nucleotides typically found in naturally occurring DNA or RNA (which can include modifications such as methylated nucleobases), joined by phosphodiester bonds. In some embodiments, a nucleic acid may comprise one or more non-standard nucleotides, which may be naturally occurring or non-naturally occurring (i.e., artificial; not found in nature) in various embodiments and/or may contain a modified sugar or modified backbone linkage. Nucleic acid modifications (e.g., base, sugar, and/or backbone modifications), non-standard nucleotides or nucleosides, etc., such as those known in the art as being useful in the context of RNA interference (RNAi), aptamer, CRISPR technology, polypeptide production, reprogramming, or antisense-based molecules for research or therapeutic purposes may be incorporated in various embodiments. Such modifications may, for example, increase stability (e.g., by reducing sensitivity to cleavage by nucleases), decrease clearance in vivo, increase cell uptake, or confer other properties that improve the translation, potency, efficacy, specificity, or otherwise render the nucleic acid more suitable for an intended use. Various non-limiting examples of nucleic acid modifications are described in, e.g., Deleavey G F, et al., Chemical modification of siRNA. Curr. Protoc. Nucleic Acid Chem. 2009; 39:16.3.1-16.3.22; Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008; U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929, 226; 5,977,296; 6,140,482; 6,455,308 and/or in PCT application publications WO 00/56746 and WO 01/14398. Different modifications may be used in the two strands of a double-stranded nucleic acid. A nucleic acid may be modified uniformly or on only a portion thereof and/or may contain multiple different modifications. Where the length of a nucleic acid or nucleic acid region is given in terms of a number of nucleotides (nt) it should be understood that the number refers to the number of nucleotides in a single-stranded nucleic acid or in each strand of a double-stranded nucleic acid unless otherwise indicated. An “oligonucleotide” is a relatively short nucleic acid, typically between about 5 and about 100 nt long.

Treating Leukemia

Acute myeloid leukemia (AML) is a malignancy of hematopoietic stem and progenitor cells that annually affects 20,000 people and claims 13,000 lives in the US alone (National Comprehensive Cancer Network (NCCN), Clinical Practice Guidelines in Oncology (2016)). New therapeutic strategies however have not yet been realized and the survival of AML patients has not improved significantly in decades. Significantly, the present inventors have discovered that inhibition of proton exporters (e.g., MCT4, NHE1) selectively eradicates and reduces the proliferation of leukemia cells, including leukemia initiating cells (LICs).

Some aspects of the present invention are directed to a method of treating leukemia in a subject in need thereof, comprising administering to the subject a therapeutically effective amount of an agent that inhibits the activity or expression of a proton exporter.

In some embodiments, the leukemia is selected from the group consisting of acute myeloid leukemia (AML), myelodysplastic syndrome (MDS), acute lymphoblastic leukemia (ALL) and chronic lymphocytic leukemia (CLL). In some embodiments, the leukemia is acute myeloid leukemia (AML). As used herein, “acute myeloid leukemia” encompasses all forms of acute myeloid leukemia and related neoplasms according to the World Health Organization (WHO) classification of myeloid neoplasms and acute leukemia, including all of the following subgroups in their relapsed or refractory state: Acute myeloid leukemia with recurrent genetic abnormalities, such as AML with t(8;21)(q22;q22); RUNX1-RUNX1T1, AML with inv(16)(p13.1q22) or t(16;16)(p13.1;q22); CBFB-MYH11, AML with t(9;11)(p22;q23); MLLT3-MLL, AML with t(6;9)(p23;q34); DEK-NUP214, AML with inv(3)(q21 q26.2) or t(3;3)(q21;q26.2); RPN1-EVI1, AML (megakaryoblastic) with t(1;22)(p13;q13); RBM15-MKL1, AML with mutated NPM1, AML with mutated CEBPA; AML with myelodysplasia-related changes; therapy-related myeloid neoplasms; AML, not otherwise specified, such as AML with minimal differentiation, AML without maturation, AML with maturation, acute myelomonocytic leukemia, acute monoblastic/monocytic leukemia, acute erythroid leukemia (e.g., pure erythroid leukemia, erythroleukemia, erythroid/myeloid), acute megakaryoblastic leukemia, acute basophilic leukemia, acute panmyelosis with myelofibrosis; myeloid sarcoma; myeloid proliferations related to Down syndrome, such as transient abnormal myelopoiesis or myeloid leukemia associated with Down syndrome; and blastic plasmacytoid dendritic cell neoplasm.

In some embodiments of the invention, the agent is administered to a subject and reduces or eliminates the likelihood of developing leukemia (e.g., AML). In some embodiments, the subject has an increased risk of developing leukemia (e.g., AML). Several inherited genetic disorders and immunodeficiency states are associated with an increased risk of AML. These include disorders with defects in DNA stability, leading to random chromosomal breakage, such as Bloom's syndrome, Fanconi's anemia, Li-Fraumeni kindreds, ataxia-telangiectasia, and X-linked agammaglobulinemia. In some embodiments, the subject has increased risk of developing leukemia (e.g., AML) due to advanced age (e.g., over about 60, 65, 70, 75, 80, 85 years or more). In some embodiments, the subject has already been treated for leukemia (e.g., AML) and is in relapse. In some embodiments, the subject is treated by the methods of the invention immediately (e.g., within about 1 day, 2 days, 3 days, 4 days, 1 week, 2 weeks, 3 weeks, 1 month) after induction chemotherapy.

In some embodiments, administration of the agent reduces the risk of developing leukemia (e.g., AML) for about 3 months, 6 months, 9 months, 1 year, 2 years, 3 years, 4 years, 5 years, 7 years, 10 years, 15 years or more.

In some embodiments, the agent comprises a protein, nucleic acid, or small molecule as described herein. In particular embodiments, the agent is an MCT4 inhibitor. In some embodiments, the MCT4 inhibitor is acriflavine. In some embodiments, the MCT4 inhibitor is not acriflavine. In some embodiments, the MCT4 inhibitor is AZD0095. In some embodiments, the MCT4 inhibitor is not AZD0095. In some embodiments, the MCT4 inhibitor is bindarit. In some embodiments, the MCT4 inhibitor is not bindarit. In some embodiments, the MCT4 inhibitor is AX93. In some embodiments, the MCT4 inhibitor is not AZ93. In some embodiments, the MCT4 inhibitor is shRNA or an interfering RNA.

In some embodiments, the proton exporter is a Monocarboxylate Transporter (e.g., MCT1, MCT2, MCT3, MCT4, MCT8, MCT9). In some embodiments, the proton exporter is Monocarboxylate Transporter 4 (MCT4). In some embodiments, the agent does not inhibit Monocarboxylate Transporter 1 (MCT1) activity or expression. In some embodiments, the subject is administered an agent that preferentially inhibits MCT4 activity or expression over MCT1 activity or expression. In some embodiments, preferentially inhibits means that the agent inhibits MCT4 at least 2-fold, 3-fold, 4-fold, 5-fold, 10-fold, 20-fold, or 50-fold more than MCT1.

In some embodiments, the proton exporter is Sodium-Hydrogen Antiporter 1 (NHE1), also known as sodium/hydrogen exchanger 1 or SLC9A1 (SoLute Carrier family 9A1). In some embodiments, the agent is a selective inhibitor of NHE1. In some embodiments, the agent is Rimeporide, Cariporide, HMA (5-(N,N-hexamethylene)-amiloride), Phx-3 (2-aminophenoxazine-3-one), or Compound 9t (5-aryl-4-(4-(5-methyl-1H-imidazol-4-yl) piperididn-1-yl)pyrimidine analog).

In some embodiments, administration of the agent inhibits the growth, viability or clonogenic ability of leukemia cells by about 2-fold, 3-fold, 4-fold, 5-fold, 10-fold, 20-fold, 50-fold, or more. In some embodiments, administration of the agent inhibits the growth, viability or clonogenic ability of leukemia cells by about 10%, 20%, 30%, 40%, 50%, 60%, 70%, 80%, 90%, 95%, 99% or more. In some embodiments, administration of the agent inhibits the growth, viability or clonogenic ability of leukemia initiating cells (LICs). In some embodiments, administration of the agent inhibits the growth, viability or clonogenic ability of LICs by about 2-fold, 3-fold, 4-fold, 5-fold, or more. In some embodiments, administration of the agent inhibits the growth, viability or clonogenic ability of LICs by about 10%, 20%, 30%, 40%, 50%, 60%, 70%, 80%, 90%, 95%, 99%, 99.9%, 99.99%, 99.999% or more. In some embodiments, administration of the agent eradicates or substantially eradicates LICs.

Without wishing to be bound by theory, it is expected that the amount of leukemic cells (e.g., LICs) eradicated, reduced, or inhibited in any particular population of cells is proportional to the concentration of the agent to which the population of cells has been exposed. In some instances, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or as much as 100% of the leukemic cells (e.g., LICs) in the population of cells are eradicated, reduced, or inhibited by exposure to or contact with the agent. In some embodiments, at least 20% of the leukemic cells (e.g., LICs) in the population of cells are eradicated, reduced, or inhibited. In some embodiments, at least 50% of the leukemic cells (e.g., LICs) in the population of cells are eradicated, reduced, or inhibited. In some embodiments, at least 70% of the leukemic cells (e.g., LICs) in the population of cells are eradicated, reduced, or inhibited. In some embodiments, all of the leukemic cells (e.g., LICs) in the population of cells are eradicated, reduced, or inhibited.

In some embodiments, administration of the agent reduces the risk of developing leukemia by about 2-fold, 3-fold, 4-fold, 5-fold, or more. In some embodiments, the administration of the agent reduces the risk of developing leukemia by about 10%, 20%, 30%, 40%, 50%, 60%, 70%, 80%, 90%, 95%, 99% or more.

In some embodiments, administration of the agent reduces the risk of relapse by about 2-fold, 3-fold, 4-fold, 5-fold, or more. In some embodiments, the administration of the agent reduces the risk of relapse by about 10%, 20%, 30%, 40%, 50%, 60%, 70%, 80%, 90%, 95%, 99% or more.

In some embodiments, the agent preferentially inhibits the growth, viability or clonogenic ability of leukemia cells in the bone marrow. In some embodiments, the agent inhibits the growth, viability or clonogenic ability of leukemia cells in the bone marrow at least 1.5-fold, 2-fold, 3-fold, 4-fold, or 5-fold more that leukemia cells not in the bone marrow.

In some embodiments, the leukemia cells exhibit increased intracellular pH (pHi) as compared to non-leukemic blood cells. In some embodiments, the pHi of the leukemia cells is at least about 7.2, 7.3, 7.4, 7.5, 7.6, or 7.7. In some embodiments, the pHi of the leukemia cells is about 7.3-7.7. In some embodiments, the pHi of the leukemia cells is about 7.4-7.6. In some embodiments, the pHi of the leukemia cells is about 7.5. In some embodiments, the pHi of the non-leukemic blood cells is about 7.0, 7.1, or 7.2. In some embodiments, the pHi of the non-leukemic blood cells is about 7.0-7.2. In some embodiments, the pHi of the non-leukemic blood cells is about 7.1.

In some embodiments, the leukemic cells exhibit increased transcriptional activation marks (e.g., H3K27ac and H3K4me3) in the MCT4 promoter region as compared to non-leukemic blood cells. In some embodiments, the leukemic cells have at least about 2-fold, 3-fold, 4-fold, 5-fold, 7-fold, 10-fold, 20-fold, 30-fold, 50-fold, 70-fold, or more transcriptional activation marks in the MCT4 promoter region as compared to non-leukemic blood cells.

In some embodiments, the leukemic cells exhibit increased transcriptional activation marks (e.g., H3K27ac and H3K4me3) in the NHE1 promoter region as compared to non-leukemic blood cells. In some embodiments, the leukemic cells have at least about 2-fold, 3-fold, 4-fold, 5-fold, 7-fold, 10-fold, 20-fold, 30-fold, 50-fold, 70-fold, or more transcriptional activation marks in the NHE1 promoter region as compared to non-leukemic blood cells.

In some embodiments, the leukemic cells exhibit increased MCT4 expression or activity. In some embodiments, the leukemic cells have about 1.5-fold, 2-fold, 3-fold, 4-fold, 5-fold, or more MCT4 expression or activity than non-cancerous blood cells. In some embodiments, the leukemic cells have about 10%, 20%, 30%, 40%, 50%, 60%, 70%, 80%, 90%, 95%, 99% or more MCT4 expression or activity than non-cancerous blood cells.

In some embodiments, the leukemic cells exhibit increased NHE1 expression or activity. In some embodiments, the leukemic cells have about 1.5-fold, 2-fold, 3-fold, 4-fold, 5-fold, or more NHE1 expression or activity than non-cancerous blood cells. In some embodiments, the leukemic cells have about 10%, 20%, 30%, 40%, 50%, 60%, 70%, 80%, 90%, 95%, 99% or more NHE1 expression or activity than non-cancerous blood cells.

In some embodiments, the agent does not inhibit the growth, viability, or clonogenic ability of non-leukemic blood cells. It should be appreciated by those skilled in the art that the methods described herein preferably selectively affect leukemic cells without affecting normal cells (e.g., leukocytes) in the population of cells. In some embodiments, leukemic cells are selectively eradicated without eradicating, or in certain aspects minimally eradicating, normal leukocytes in the population of cells. For example, the leukemic cells are selectively eradicated without eradicating, or in certain embodiments, minimally eradicating, normal bone marrow leukocytes or normal peripheral blood leukocytes, including without limitation, stem and progenitors, bone marrow mononuclear cells, myeloblasts, neutrophils, NK cells, macrophages, granulocytes, monocytes, and lineage−/cKit+/Sca1+ (LKS) cells. In some embodiments, the amount or activity of leukemic cells in a population of cells is selectively decreased without decreasing the amount or activity of normal leukocytes in the population. In some embodiments, proliferation of leukemic cells is selectively inhibited in a population of cells without inhibiting, or minimally inhibiting proliferation of normal leukocytes in the population. In some embodiments, the agent inhibits the growth, viability or clonogenic ability of leukemia cells at least 1.5-fold, 2-fold, 3-fold, 4-fold, 5-fold, 10-fold, 20-fold, 50-fold or more than normal leukocytes. In some embodiments, the methods described herein can be used to increase the number of normal leukocytes in a population of cells by selectively reducing the number, activity, and/or proliferation of leukemic cells in the population of cells.

In some embodiments, the subject is administered a second anti-cancer (anti-leukemia) agent. Chemotherapeutic agents useful in methods disclosed herein include, but are not limited to, alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, bendamustine, chlorozotocin, fotemustine, lomustine, nimustine, ranimustine; antibiotics such as aclacinomysins, actinomycin, authramycin, azaserine, bleomycins, dactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel and docetaxel; chlorambucil; gemcitabine; 6-thioguanine; mercaptopurine; platinum analogs such as cisplatin and carboplatin; vinblastine; platinum; etoposide; ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine; retinoic acid; esperamicins; capecitabine; and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide, and irinotecan, as well as pharmaceutically acceptable salts, acids, or derivatives of any of these. In some embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain e embodiments, the antimitotic agent comprises a vinca alkaloid, such as vincristine, binblastine, vinorelbine, or vindesine, or pharmaceutically acceptable salts, acids, or derivatives thereof. In some embodiments, the second agent is a second proton exporter inhibitor.

In some embodiments, the one or more second anti-cancer agents are cytarabine and an anthracycline. In some embodiments, the one or more anti-cancer agents are doxorubicin hydrochloride and cytarabine.

In some embodiments, the subject is administered a second agent selected from a pro-apototic agent (e.g., venetoclax), an agent that enhances non-caspase dependent cell death (e.g., GPX4 inhibitor), an immunotherapeutic agent (e.g., CD47 inhibitor, checkpoint inhibitor), an antibody drug conjugate, a Bi-specific T-cell engager (BiTE), dual ipilimumab and nivolumab therapy (DART), or an immunologic cell therapy (e.g., NK-CAR, CAR-T). The pro-apopototic agents are not limited and may be any suitable agent. See, e.g., Baig, S et al. “Potential of apoptotic pathway-targeted cancer therapeutic research: Where do we stand?.” Cell death & disease vol. 7,1 e2058. 14 Jan. 2016, incorporated herein by reference. The agents that enhances non-caspase dependent cell death (e.g., GPX4 inhibitors) are not limited and may be any suitable agent. See, e.g., Hangauer et al. Drug-tolerant persister cancer cells are vulnerable to GPX4 inhibition. Nature 2017 Nov. 1, incorporated herein by reference. The immunotherapeutic agents are not limited and may be any suitable agent. In some embodiments, the immunotherapeutic is Ipilimumab (Yervoy), Nivolumab (Opdivo), Pembrolizumab (Keytruda), Atezolizumab (Tecentriq), Avelumab (Bavencio), Durvalumab (Imfinzi), an interferon, or an interleukin. See also, e.g., Ventola C L. Cancer Immunotherapy, Part 1: Current Strategies and Agents. P T. 2017; 42(6):375-383, incorporated herein by reference. The antibody drug conjugates are not limited and may be any suitable agent. See also, e.g., Lambert et al., Antibody-Drug Conjugates for Cancer Treatment, Annu Rev Med. 2018 Jan. 29; 69:191-207. The immunologic cell therapies are not limited and may be any suitable agent.

In some embodiments, the one or more anti-cancer agents are administered prior to, simultaneously with, or after administration of the compositions of the invention. In some embodiments, the one or more anti-cancer agents are administered about 1, 2, 3, 4, 5, 6, 7, 8, 9, 10, 15, 20, 25, 30, 60, 90, 120 days prior to or after the administration of the composition.

In some embodiments, the second anti-cancer agent is a glycolysis inhibitor, a histone deacetylase inhibitor, or a pentose phosphate pathway (PPP) inhibitor. The glycolysis inhibitor is not limited and may be any suitable glycolysis inhibitor known in the art. In some embodiments, the glycolysis inhibitor is 2-Deoxy-D-Glucose, 3-Bromopyruvic acid, 6-Aminonicotinamide, Lonidamine, Oxythiamine Chloride Hydrochloride, Shikonin, Imatinib, 5-Thioglucose, or Glufosfamide. The histone deacetylase inhibitor is not limited and may be any suitable histone deacetylase inhibitor known in the art. In some embodiments, the histone deacetylase inhibitor is FK228, AN-9, MS-275, CI-994, LAQ-824, SAHA, G2M-777, PXD-101, LBH-589, MGCD-0103, MK0683, pyroxamide, sodium phenylbutyrate, CRA-024781, Belinostat; (i.e. PXD101), MS-275 (i.e.,Entinostat; MS-27-275), Vorinostat (i.e. suberoylanilide hydroxamic acid (SAHA); Zolinza), Mocetinostat (i.e. MGCD0103), SB939 (i.e. Pracinostat), Rocilinostat (i.e. ACY-1215), or derivatives, salts, metabolites, prodrugs, and stereoisomers thereof. The PPP inhibitor is not limited and may be any suitable PPP inhibitor known in the art. In some embodiments, the PPP inhibitor is 6-aminonicotinamide (6-AN), epiandrosterone (EPI), or dehydroepiandrosterone (DHEA).

Inhibiting Cancer by Decreasing pHi

Some aspects of the present invention are directed to a method of inhibiting the growth, viability, or clonogenic ability of a cancer cell, comprising contacting the cancer cell with an agent that decreases the intracellular pH (pHi) of the cancer cell.

In some embodiments, administration of the agent inhibits the growth, viability or clonogenic ability of the cancer cell by about 2-fold, 3-fold, 4-fold, 5-fold, 10-fold, 20-fold, 50-fold, or more. In some embodiments, administration of the agent inhibits the growth, viability or clonogenic ability of the cancer cell by about 10%, 20%, 30%, 40%, 50%, 60%, 70%, 80%, 90%, 95%, 99%, 99.9%, or even 100%.

In some embodiments, the agent decreases the pHi of the cancer cell to about 7.4, 7.3, 7.2, 7.1, or 7.0. In some embodiments, the agent decreases the pHi of the cancer cell to about 7.1. In some embodiments, the agent decreases the pHi by about 1.1-fold, 1.2-fold, 1.3-fold, 1.4-fold, 1.5-fold, 1.6-fold, 1.7-fold, 1.8-fold, 2-fold, 3-fold, 4-fold, 5-fold, or more. Decreasing pHi by 10-fold corresponds to decreasing pHi by 1, e.g., from pH 8 to pH 7.

The cancer cell is not limited. In some embodiments, the cancer cell is from breast cancer; biliary tract cancer; bladder cancer; brain cancer (e.g., glioblastomas, medulloblastomas); cervical cancer; choriocarcinoma; colon cancer; endometrial cancer; esophageal cancer; gastric cancer; hematological neoplasms including acute lymphocytic leukemia and acute myelogenous leukemia; T-cell acute lymphoblastic leukemia/lymphoma; hairy cell leukemia; chronic lymphocytic leukemia, chronic myelogenous leukemia, multiple myeloma; adult T-cell leukemia/lymphoma; intraepithelial neoplasms including Bowen's disease and Paget's disease; liver cancer; lung cancer; lymphomas including Hodgkin's disease and lymphocytic lymphomas; neuroblastoma; melanoma, oral cancer including squamous cell carcinoma; ovarian cancer including ovarian cancer arising from epithelial cells, stromal cells, germ cells and mesenchymal cells; neuroblastoma, pancreatic cancer; prostate cancer; rectal cancer; sarcomas including angiosarcoma, gastrointestinal stromal tumors, leiomyosarcoma, rhabdomyosarcoma, liposarcoma, fibrosarcoma, and osteosarcoma; renal cancer including renal cell carcinoma and Wilms tumor; skin cancer including basal cell carcinoma and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullary carcinoma.

In some embodiments, the cancer cell exhibits increased intracellular pH (pHi) as compared to a non-cancer cell. In some embodiments, the pHi of the cancer cell is at least about 7.2, 7.3, 7.4, 7.5, 7.6, or 7.7. In some embodiments, the pHi of the cancer cell is about 7.3-7.7. In some embodiments, the pHi of the cancer cell is about 7.4-7.6. In some embodiments, the pHi of the cancer cell is about 7.5. In some embodiments, the pHi of the non-cancer cell is about 7.0, 7.1, or 7.2. In some embodiments, the pHi of the non-cancer cell is about 7.0-7.2. In some embodiments, the pHi of the non-cancer cell is about 7.1.

In some embodiments, the cancer is glycolysis dependent. In some embodiments, the cancer is not OXPHOS-dependent.

In some embodiments, the cancer cell comprises an oncogenic protein having increased activity at increased pHi. The oncogenic protein having increased activity at increased pHi is not limited. In some embodiments, the oncogenic protein is an oncogenic protein disclosed in White, et al. (2017) “Cancer-associated arginine-to-histidine mutations confer a gain in pH sensing to mutant proteins,” Sci Signal 10.

In some embodiments, the cancer cell exhibits increased activity or expression of a proton exporter (e.g., MCT4, NHE1) as compared to a non-cancer cell. In some embodiments, the cancer cells have about 2-fold, 3-fold, 4-fold, 5-fold, or more proton exporter (e.g., MCT4, NHE1) expression or activity than non-cancerous cells. In some embodiments, the cancer cells have about 10%, 20%, 30%, 40%, 50%, 60%, 70%, 80%, 90%, 95%, 99% or more proton exporter (e.g., MCT4, NHE1) expression or activity than non-cancerous cells.

In some embodiments, the proton exporter is Monocarboxylate Transporter 4 (MCT4). In some embodiments, the proton exporter is NHE1. The agent is not limited and may be any agent described herein. In some embodiments, the agent inhibits the activity or expression of the proton exporter (e.g., MCT4, NHE1) in the cancer cell. In some embodiments, the agent does not inhibit MCT4 activity or expression. In some embodiments, the agent does not inhibit MCT1 activity or expression. In some embodiments, the agent preferentially inhibits means that the agent inhibits MCT4 at least 2-fold, 3-fold, 4-fold, 5-fold, 10-fold, 20-fold, or 50-fold more than MCT1.

In some embodiments, the agent preferentially inhibits the growth, viability, or clonogenic ability of a cancer cell in a low oxygen environment (e.g., in hypoxic tissue). In some embodiments, the agent inhibits the growth, viability or clonogenic ability of a cancer cell in a low oxygen environment at least 1.5-fold, 2-fold, 3-fold, 4-fold, or 5-fold more that a cancer cell not in a low oxygen environment (e.g., in hypoxic tissue). In some embodiments, the agent does not inhibit, or minimally inhibits, the growth or viability of non-cancerous cells. In some embodiments, the cancer is leukemia.

In some embodiments, the agent is administered to a subject having cancer. In some embodiments, the subject is administered a second anti-cancer agent. The second cancer agent may be any anti-cancer agent (e.g., anti-leukemic agent) described herein. In some embodiments, the second anti-cancer agent is a glycolysis inhibitor, a histone deacetylase inhibitor, or a pentose phosphate pathway (PPP) inhibitor. In some embodiments, the subject is administered a second agent, as disclosed herein, selected from a pro-apototic agent (e.g., venetoclax), an agent that enhances non-caspase dependent cell death (e.g., GPX4 inhibitor), an immunotherapeutic agent (e.g., CD47 inhibitor, checkpoint inhibitor), an antibody drug conjugate, a Bi-specific T-cell engager (BiTE), dual ipilimumab and nivolumab therapy (DART), or an immunologic cell therapy (e.g., NK-CAR, CAR-T).

Preventing Leukemia

Some aspects of the present invention are directed to a method of preventing, delaying, reducing the likelihood of relapse of, or reducing the likelihood of leukemia in a subject in need thereof, comprising administering to the patient a therapeutically effective amount of a Monocarboxylate Transporter 4 (MCT4) inhibitor. The Monocarboxylate Transporter 4 (MCT4) inhibitor may be an agent as described herein. The subject is not limited and may be any subject described herein. The administration is not limited and may be by any method or at any interval described herein.

In some embodiments, administration of the inhibitor reduces the risk of developing leukemia by about 2-fold, 3-fold, 4-fold, 5-fold, or more. In some embodiments, the administration of the inhibitor reduces the risk of developing leukemia by about 10%, 20%, 30%, 40%, 50%, 60%, 70%, 80%, 90%, 95%, 99% or more.

In some embodiments, administration of the inhibitor reduces the risk of relapse by about 2-fold, 3-fold, 4-fold, 5-fold, or more. In some embodiments, the administration of the inhibitor reduces the risk of relapse by about 10%, 20%, 30%, 40%, 50%, 60%, 70%, 80%, 90%, 95%, 99% or more.

In some embodiments, administration of the inhibitor reduces the risk of developing leukemia (e.g., AML) for about 3 months, 6 months, 9 months, 1 year, 2 years, 3 years, 4 years, 5 years, 7 years, 10 years, 15 years or more.

In some embodiments, the subject has one or more risk factors associated with the development of leukemia. In some embodiments, the one or more risk factors include advanced age or the presence of a gene mutation. In some embodiments, a subject is at risk of developing acute myeloid leukemia based on a genetic mutation useful as a diagnostic or prognostic marker of myeloid neoplasms. Exemplary such markers include mutations of: JAK2, MPL, and KIT in MPN; NRAS, KRAS, NF1, and PTPN11 in MDS/MPN; NPM1, CEBPA, FLT3, RUNX1, KIT, WT1, and MLL in AML; and GATA1 in myeloid proliferations associated with Down syndrome (see Vardiman, et al., “The 2008 revision of the World Health Organization (WHO) classification of myeloid neoplasms and acute leukemia: rationale and important changes,” Blood 114(5), 937-951 (2009), incorporated herein by reference in its entirety). In some embodiments, the subject has increased risk of developing leukemia (e.g., AML) due to advanced age (e.g., over about 60, 65, 70, 75, 80 years or more).

Methods of Screening

Some aspects of the present invention are directed to a method of determining if a cancer is responsive to MCT4 inhibition therapy, comprising determining if the level of transcriptional activation marks (e.g., H3K27ac and H3K4me3) on the MCT4 promoter of the cancer cell is elevated as compared to a control non-cancerous cell. In some embodiments, the cancer cells identified as responsive to MCT4 inhibition therapy have at least about 2-fold, 3-fold, 4-fold, 5-fold, 7-fold, 10-fold, 20-fold, 30-fold, 50-fold, 70-fold, or more transcriptional activation marks in the MCT4 promoter region as compared to non-cancer control cells. The cancer is not limited and may be any cancer described herein. In some embodiments, the cancer is leukemia (e.g., AML). The method of determining the level of transcriptional activation marks is not limited and may be any suitable method. In some embodiments, the level of transcriptional activation marks on the MCT4 promoter is determined by ChIP-PCR. In some embodiments, if the level of transcriptional activation marks on the MCT4 promoter of the cancer cell is elevated, then the cancer cell is contacted with an agent as described herein.

Some aspects of the present invention are directed toward a method of determining if a cancer is responsive to MCT4 inhibition therapy, comprising determining if the level of MCT4 expression in the cancer cell is elevated as compared to a control non-cancerous cell. In some embodiments, the cancer cells identified as responsive to MCT4 inhibition therapy have about 2-fold, 3-fold, 4-fold, 5-fold, or more MCT4 expression or activity than non-cancerous control cells. In some embodiments, the cancer cells have about 10%, 20%, 30%, 40%, 50%, 60%, 70%, 80%, 90%, 95%, 99% or more MCT4 expression or activity than non-cancerous control cells. The cancer is not limited and may be any cancer described herein. In some embodiments, the cancer is leukemia (e.g., AML). In some embodiments, if the level of MCT4 expression or activity is elevated, then the cancer cell is contacted with an agent as described herein.

Some aspects of the present invention are directed toward a method of determining if a cancer is responsive to NHE1 inhibition therapy, comprising determining if the level of NHE1 expression in the cancer cell is elevated as compared to a control non-cancerous cell. In some embodiments, the cancer cells identified as responsive to NHE1 inhibition therapy have about 2-fold, 3-fold, 4-fold, 5-fold, or more NHE1 expression or activity than non-cancerous control cells. In some embodiments, the cancer cells have about 10%, 20%, 30%, 40%, 50%, 60%, 70%, 80%, 90%, 95%, 99% or more NHE1 expression or activity than non-cancerous control cells. The cancer is not limited and may be any cancer described herein. In some embodiments, the cancer is leukemia (e.g., AML). In some embodiments, if the level of NHE1 expression or activity is elevated, then the cancer cell is contacted with an agent as described herein.

Methods of Increasing Growth or Proliferation of Cells

Some aspects of the present invention are directed to a method of increasing the growth or proliferation of a cell comprising contacting the cell with an agent that increases the expression or activity of a proton exporter. The proton exporter is not limited and may be any proton exporter known in the art or described herein. In some embodiments, the proton exporter is Monocarboxylate Transporter 4 (MCT4). In some embodiments, the proton exporter is NHE1. The agent is not limited and may be a polypeptide, nucleic acid, or small molecule as described herein. In some embodiments, the agent is a nucleic acid coding for a proton exporter (e.g., MCT4, NHE1). In some embodiments, the agent is a proton exporter (e.g., MCT4, NHE1) protein or variant or derivative thereof. In some embodiments, the agent increases the growth or proliferation of a cell by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, or more as compared to a cell not contacted with the agent. In some embodiments, the agent increases the growth or proliferation of a cell by about 10%, 20%, 30%, 40%, 50%, 60%, 70%, 80%, 90%, 95%, 99% or more as to a cell not contacted with the agent.

In some embodiments, the cell is contacted with the agent in vitro (e.g., in a cell culture). In some embodiments, the agent is administered to a subject.

The cell is not limited and may be any suitable cell. In some embodiments, the cell is a hematopoietic stem or progenitor cell, myeloid hematopoietic cell, pre-osteoblast, primary tracheal epithelial cell, or primary bronchial epithelial cell. In some embodiments, the cell is a non-cancerous cell that corresponds to a cancer exhibiting increased MCT4 expression. In some embodiments, the cell is a non-cancerous cell that corresponds to a cancer exhibiting increased NHE1 expression.

### Examples

It was previously noted that AML cells exhibit alkaline pHi compared to normal blood cells (Man et al., 2014). This had been reported in a number of other cancer types and presumed to be a consequence of increased lactate generation. It was noted that inhibiting acid efflux can be toxic to cancer (Andersen et al., 2018) while increased MCT4 expression is associated with poor patient prognosis in many cancers (Bovenzi et al., 2015) including AML (The Cancer Genome Atlas (TCGA) LAML database (portal.gdc.cancer.gov)). Further, oncogenic proteins bearing specific mutations, arginine-histidine transitions, were reported to have enhanced function in increased intracellular pH (White et al., 2017). The inventors hypothesized that altered proton handling may play a more central role in cell growth and thus focused on hematopoietic cells to address this.

The distinctive metabolic features and dependencies of malignant versus normal myeloid hematopoietic cells has been defined by several laboratories (Lu et al., 2012; Wang et al., 2014). Further, targeting cell metabolism is a strategy with extensive pre-clinical and clinical experience in malignant hematopoiesis. For example, inhibiting mutant IDH clinically (DiNardo et al., 2018; Stein et al., 2017), mutant PHD3 (German et al., 2016), wildtype BCAT1 (Raffel et al., 2017), CIpP (Cole et al., 2015) or DHODH pre-clinically (Sykes et al., 2016) have all been shown to have significant activity against AML. Therefore, AML metabolism may both yield insight into novel growth control mechanisms and inform new therapeutic approaches to a disease with dismal survival statistics (Ossenkoppele and Lowenberg, 2015).

It is shown herein that increased expression of the proton exporter, MCT4, increases the proliferation of malignant and normal HSPC. It does so by elevating pHi. Notably, multiple leukemogenic alleles alter histone signatures at the MCT4 promoter, enforcing increased gene expression. MLL-AF9, a leukemogenic allele in childhood and adult AML, directly binds to BRD4 on the MCT4 promoter, increasing MCT4 expression. The elevated pHi from increased MCT4 expression results in changes in enzymatic activity, enhancing glycolytic and PPP carbon flux needed for cell growth. Particularly augmented was the activity of hexokinase, a carbon flux gatekeeper shown by others to be critical in normal and malignant cells (Tanner et al., 2018). Genetically, reducing MCT4 suppresses the growth of mouse and human AML in vitro and in vivo. MCT4-reduction causes cellular acidification and inhibition of both glycolysis and PPP. This leads to depletion of leukemia-initiating cells in mice. Our results demonstrate that proton shifts are a growth modulating feature of hematopoietic cells. Co-opting that process, mutations altering MCT4 histone marks gain a competitive growth advantage over normal cells, but may also gain a vulnerability to MCT4 inhibition.

Results

Aberrant Alkaline Intracellular pH in AML

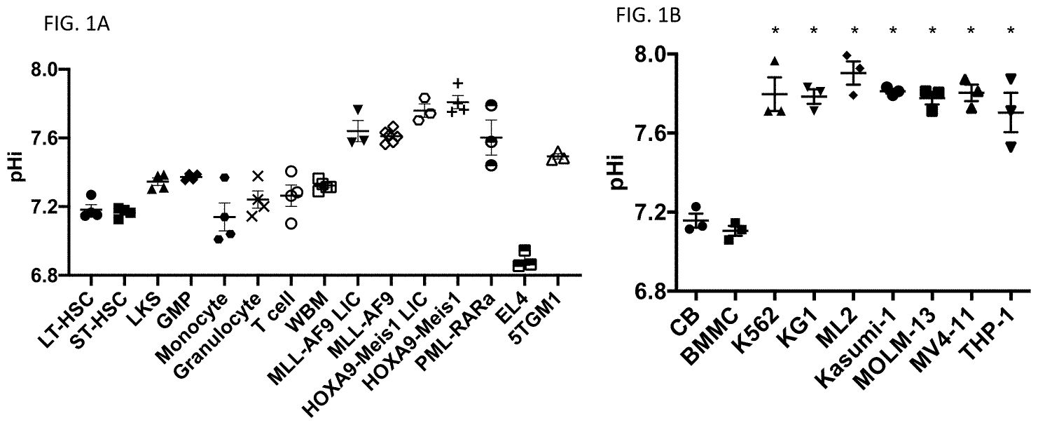

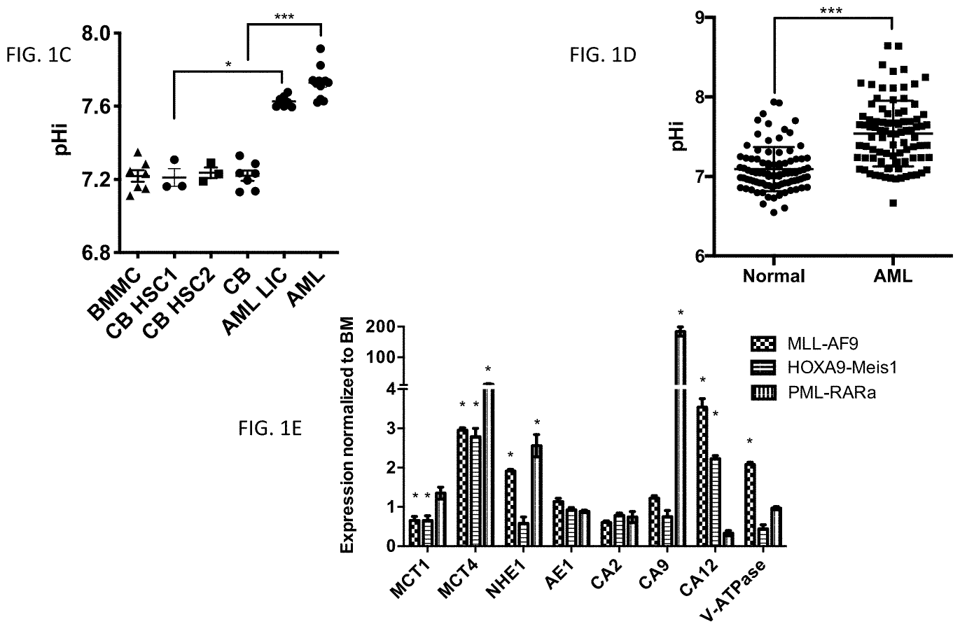

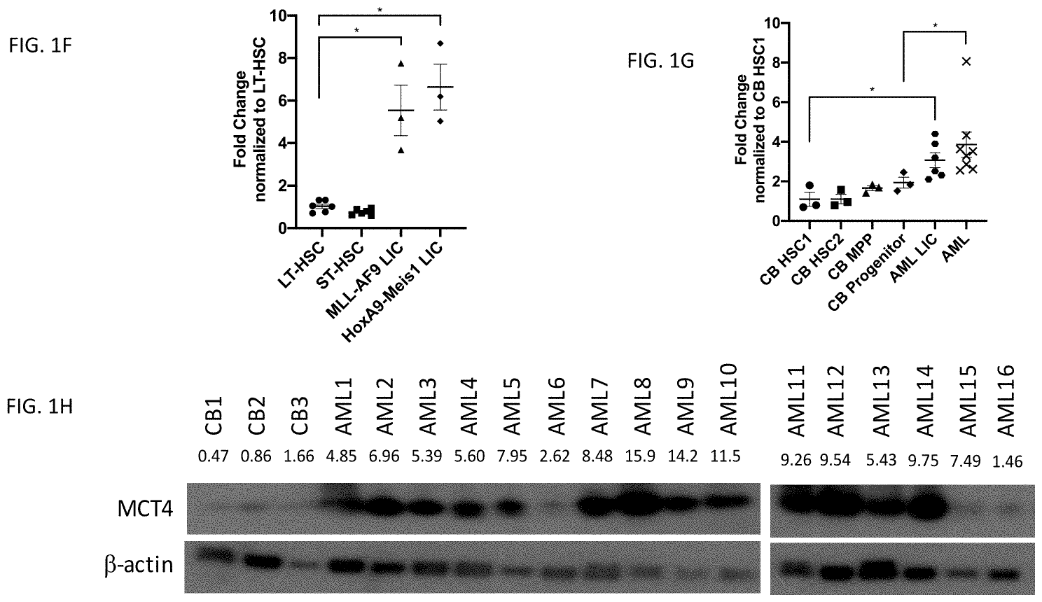

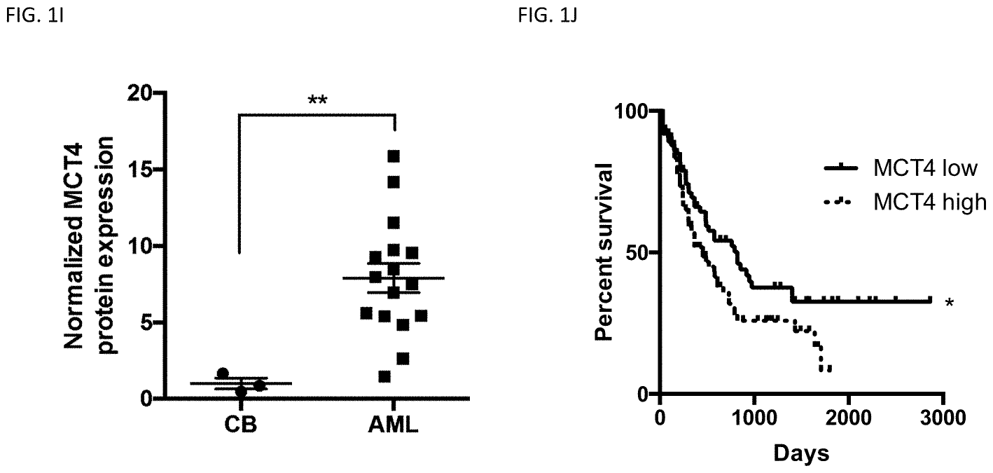

To examine the pHi in AML and normal blood cells, a pH indicator, SNARF-1 was used. The inventors compared the pHi of normal mouse whole bone marrow (WBM), LT-HSC (Lin⁻cKit+Sca-1⁺CD34⁻FLT3⁻), ST-HSC (Lin⁻cKit⁺Sca-1⁺CD34⁺FLT3⁺), LKS (Lin⁻cKit⁺Sca-1⁺), GMP (Lin⁻cKit⁺Sca-1⁻CD34⁺CD16/32⁺), monocyte (CD45⁺CD11b⁺), granulocyte (CD45⁺Gr-1⁺) and T cell (CD45⁺CD3⁺) with MLL-AF9, HoxA9-Meis1, PML-RARα AML and the LIC subpopulation (cKit+) of MLL-AF9 and HoxA9-Meis1 AML (FIG. 1A). pHi of normal blood cells ranged from 7.2 to 7.4, while that of AML was ˜7.6. Other murine hematopoietic malignancies such as multiple myeloma 5TGM1 was alkaline, while lymphoma EL4 was acidic compared to normal cells. In human, the pHi of cord blood (CB—CD34⁺), CB HSC1 (CD34⁺CD38⁻CD90⁺CD49f⁺CD45RA⁻), CB HSC2 (CD34⁺CD38⁻CD90⁺CD49f⁺CD45RA⁻) and normal bone marrow mononuclear cells (BMMC) were ˜7.2 while that of human AML and the LIC subpopulation (CD34⁺CD38⁺) were ˜7.6-7.7 (FIGS. 1B & 1C).

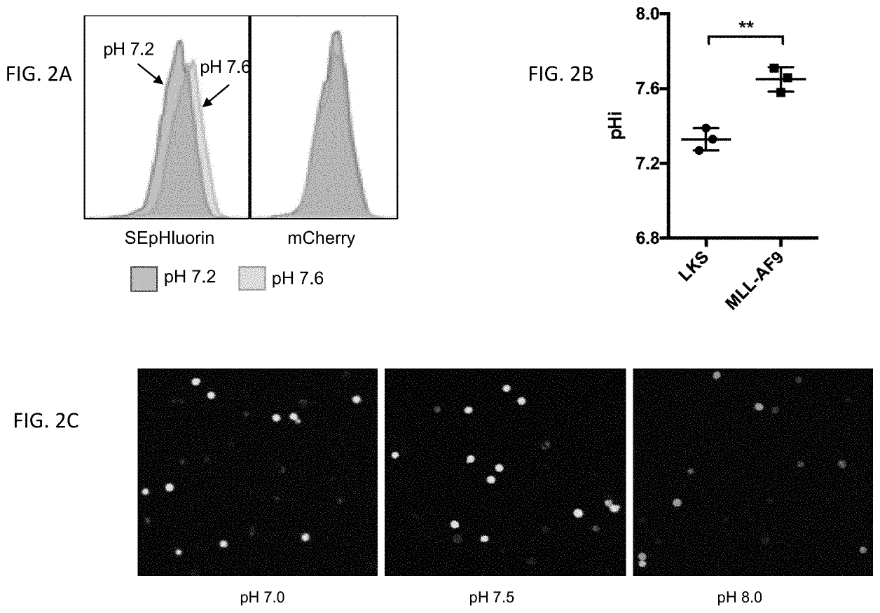

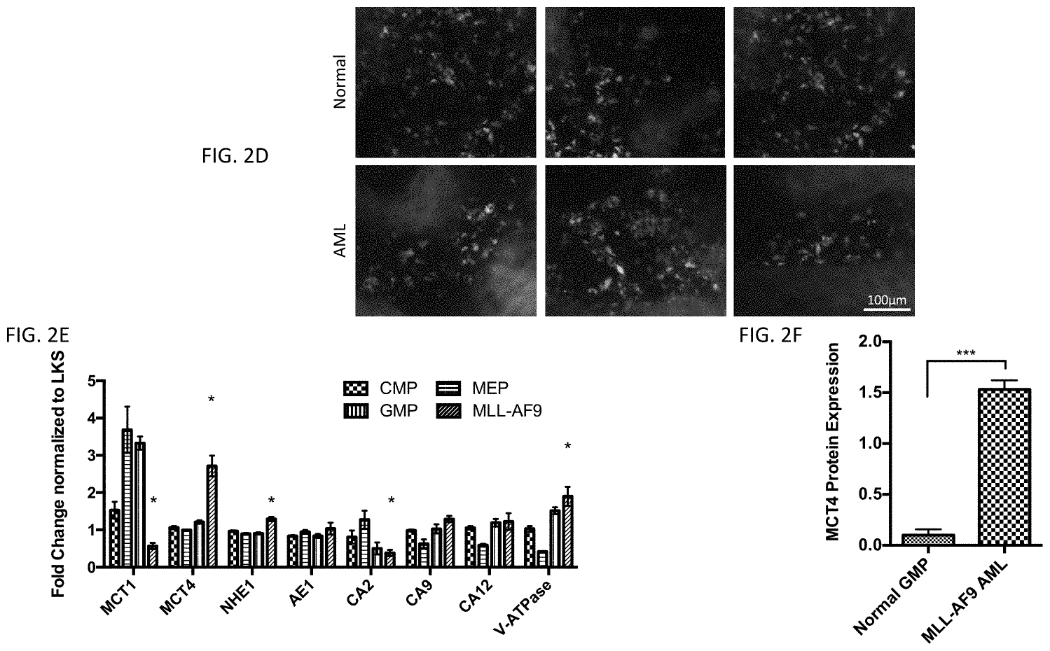

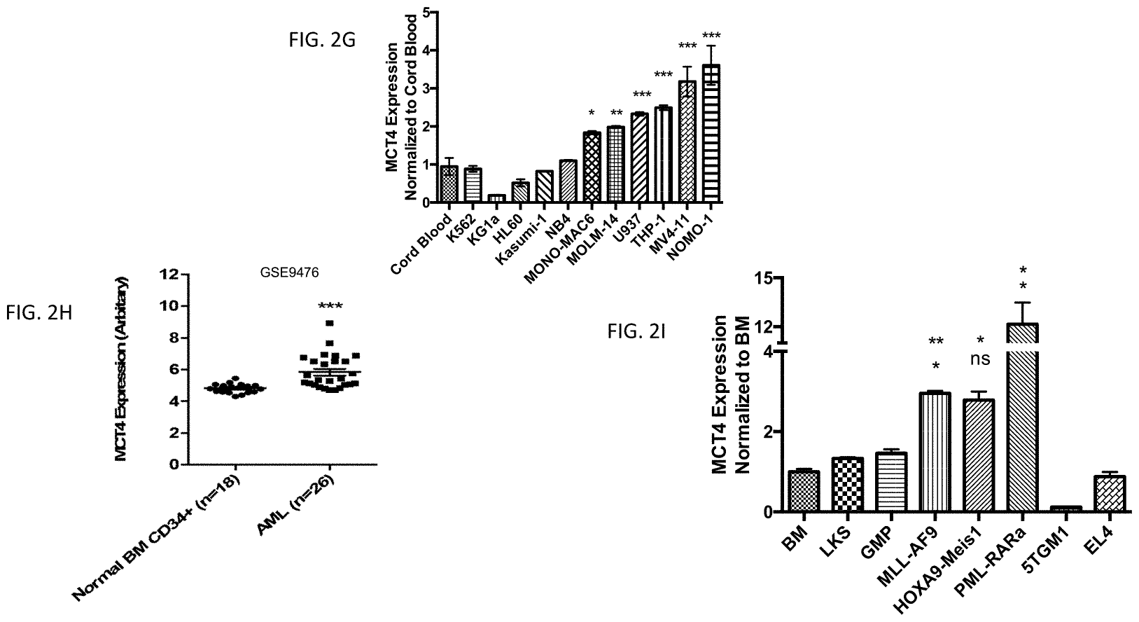

To confirm this in vivo, the inventors established a fluorescent pH reporter combining mCherry (pH insensitive) and SEpHluorin (green fluorescence decreases at acidic pH) (FIG. 2A) (Koivusalo et al., 2010). mCherry-SEpHluorin was retrovirally induced into MLL-AF9 AML and pHi was examined (FIG. 2B). The pHi examined by the reporter was comparable to that analyzed by SNARF-1 (FIG. 1A).

Normal mouse LKS and MLL-AF9 mouse AML expressing the pH reporter were transplanted into mice. The pHi of 100 cells (normal or AML) was determined (FIG. 1D) based on the calibrated reporter fluorescence (FIGS. 2C & 2D). The in vivo pHi of normal blood cells and AML were about 7.1 and 7.5 respectively, which were similar to the ex vivo pHi (FIGS. 1A & 2B). These results confirm a more alkaline pHi in AML compared to normal blood cells that is not an artifact of the isolation procedure, but is present in vivo where the cells reside.

MCT4 is Commonly Overexpressed in AML

Expression of pH regulators are often altered in cancer, though, the dependence on various pH regulator is cell-type specific (Webb et al., 2011). The inventors analyzed the gene expression profile in MLL-AF9 AML with normal mouse HSPC (GSE20377) (Krivtsov et al., 2006). Among various cancer-related pH regulators, Slc16a3 (MCT4) and Slc9a1 (NHE1) were upregulated in AML (FIG. 2E). Further, only MCT4 was consistently upregulated in other mouse AML models (FIG. 1E). The expression of MCT4 in the LIC subpopulation of mouse AML were also higher compared to mouse LT-HSC and ST-HSC (FIG. 1F). Upregulation of MCT4 protein was further confirmed in MLL-AF9 AML compared to normal GMP (FIG. 2F). MCT4 upregulation in AML was observed in human AML cell lines and primary patient samples (FIGS. 1G, 1H, 1I & 2G). These results are consistent with published AML gene expression databases including TCGA-LAML (data not shown) and GSE9476 (FIG. 2H) (Stirewalt et al., 2008). However, MCT4 was not upregulated in multiple myeloma 5TGM1 despite an elevated pHi (FIG. 2I). Therefore, AML may have a specific dependency on MCT4 as a pHi regulator. A functionally significant role for MCT4 is further supported by clinical data, TCGA-LAML, that MCT4 expression inversely correlates with prognosis in human AML (FIG. 1J: [OncoLnc: www.oncolnc.org, Cox coefficient is 0.236, p value is 0.0395]).

MCT4 Inhibition Suppresses the Growth of AML

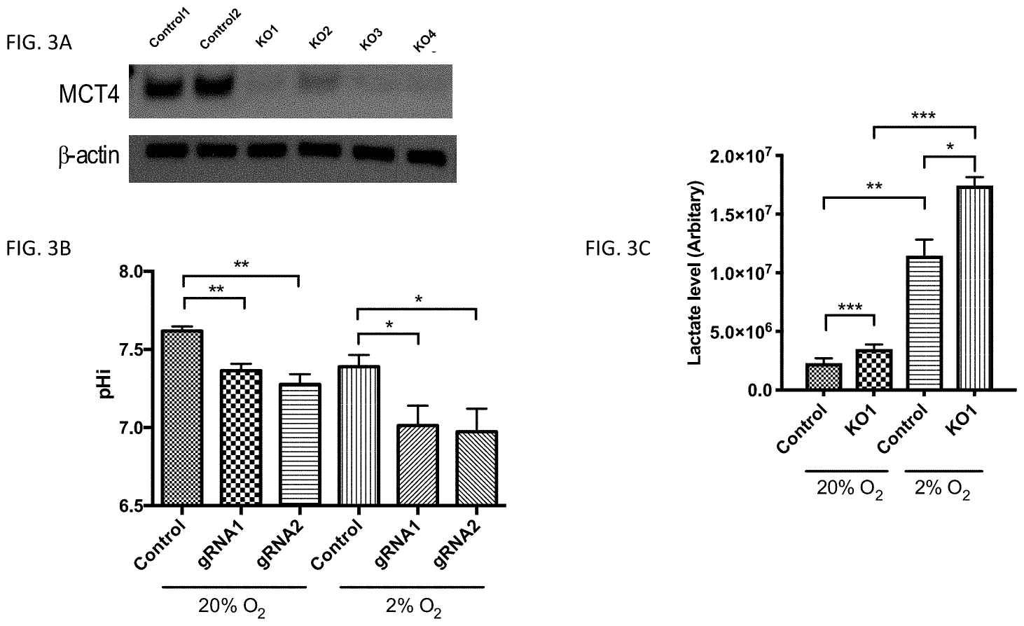

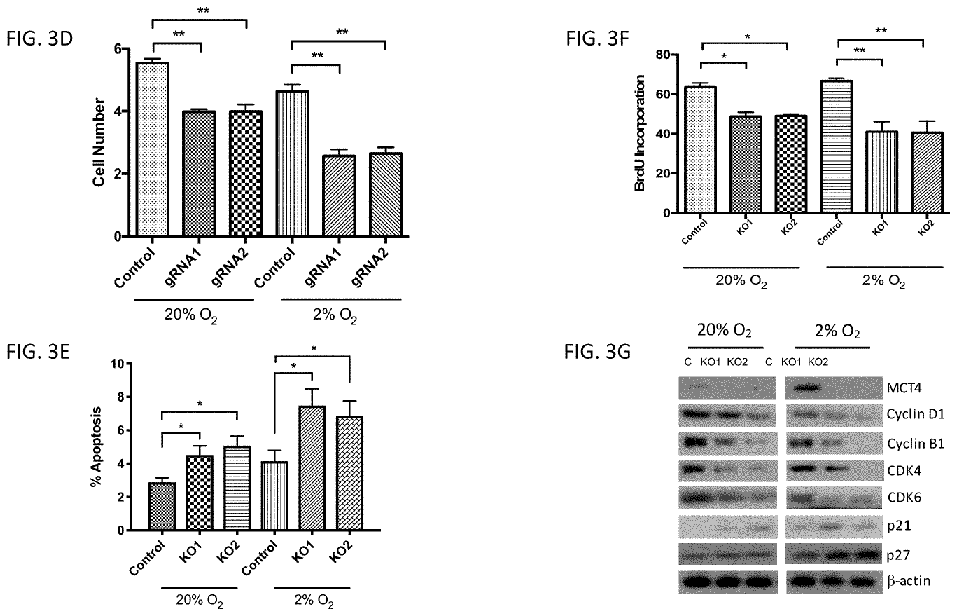

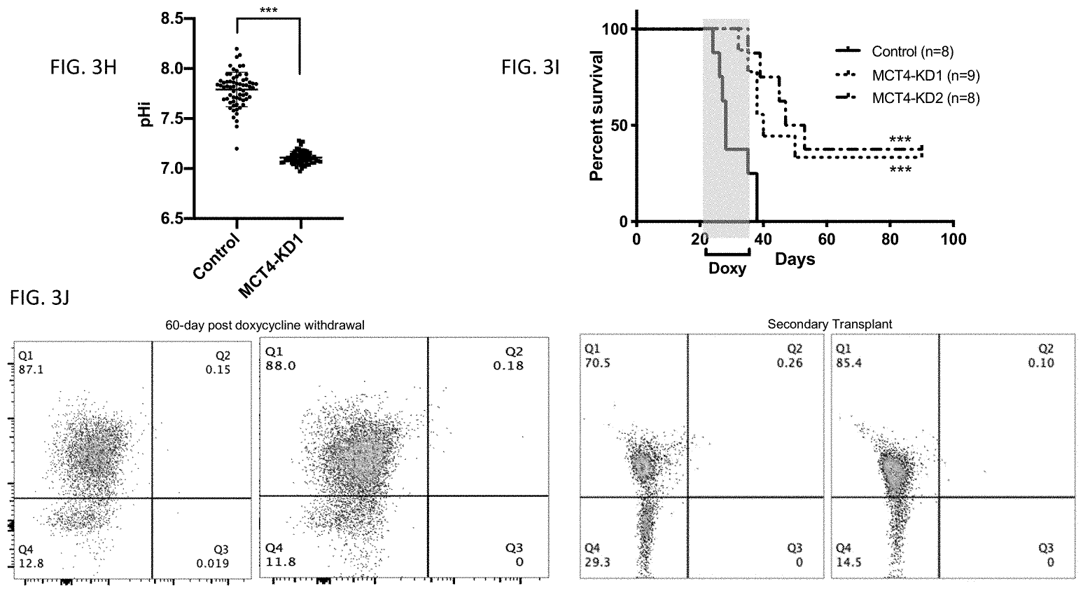

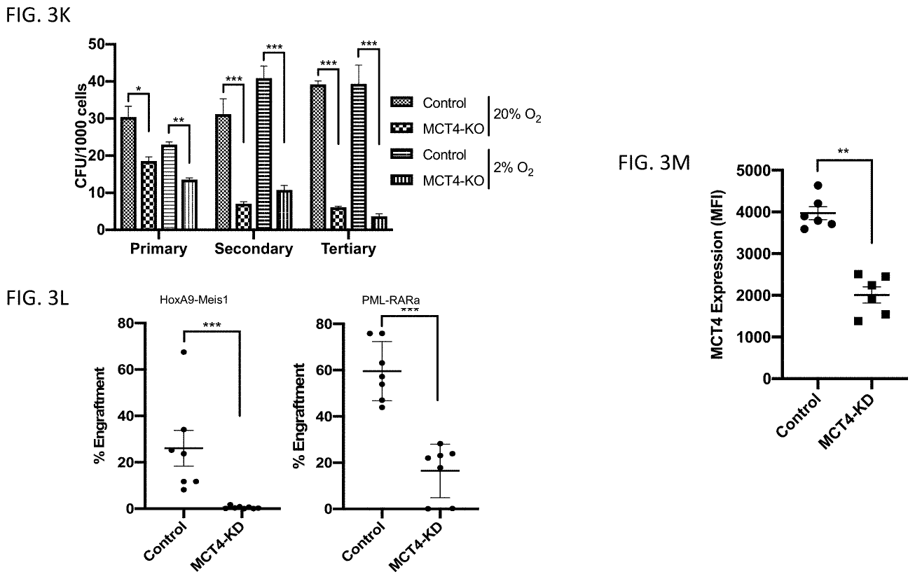

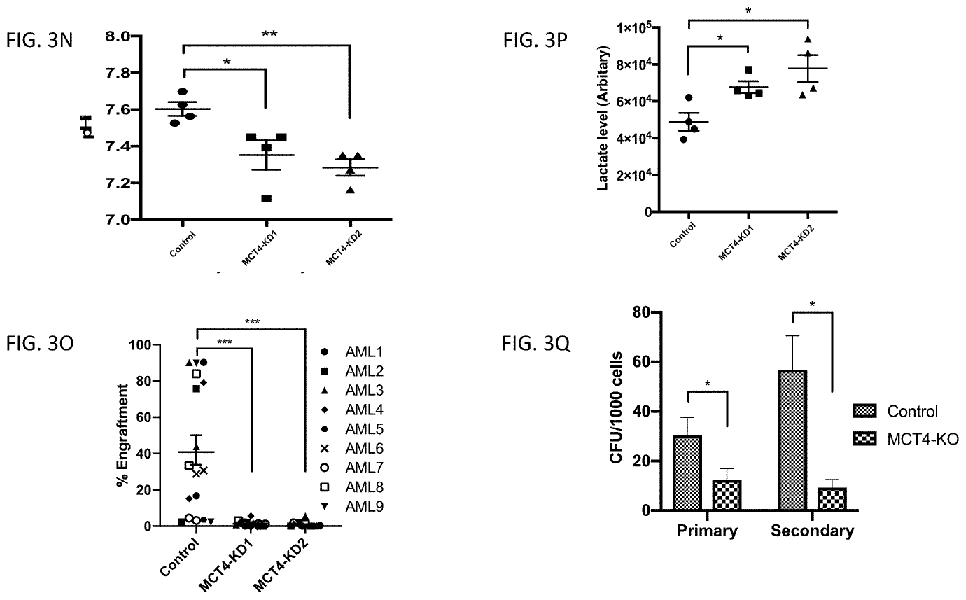

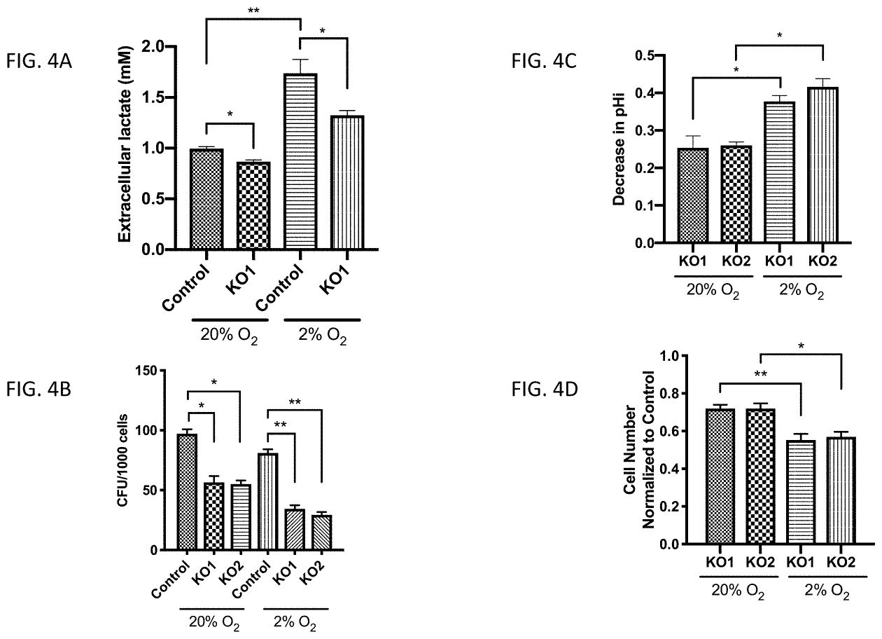

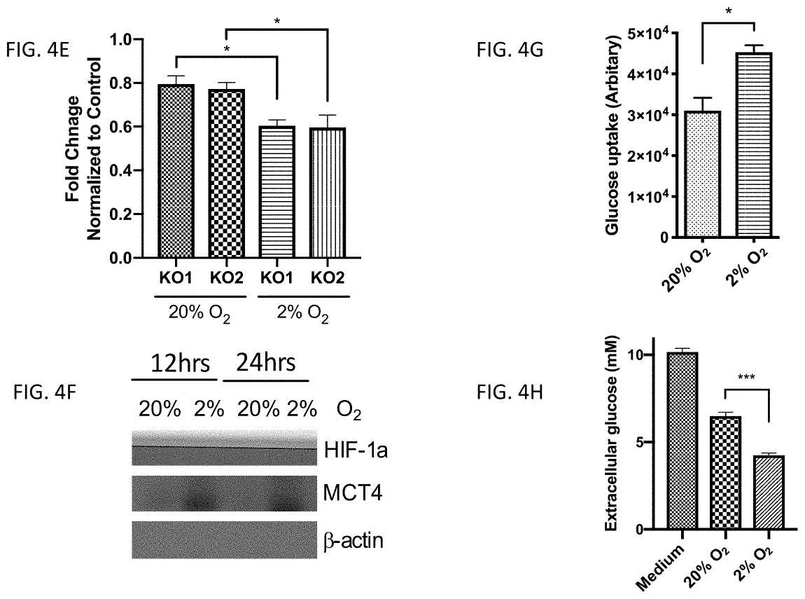

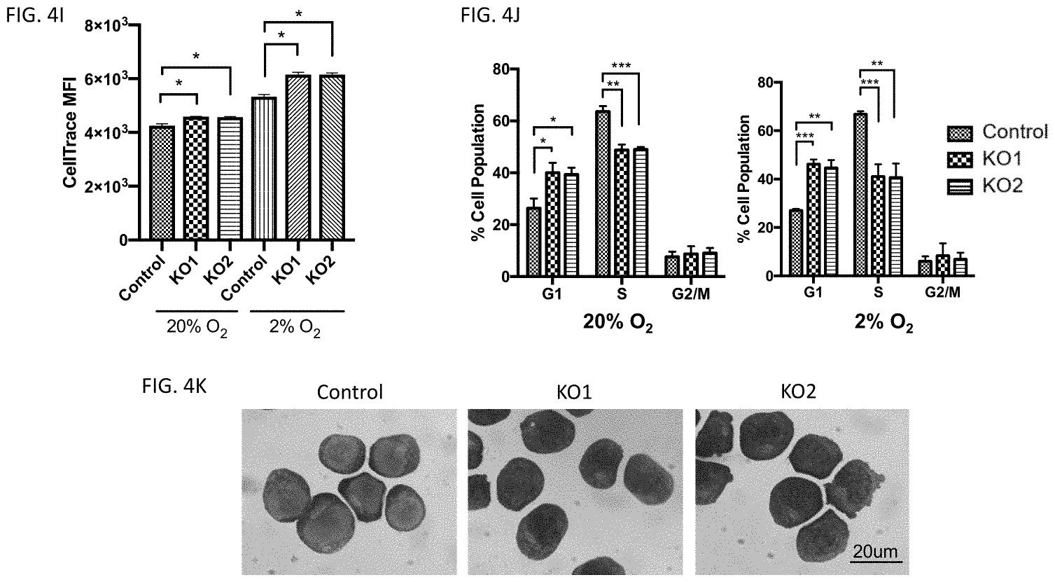

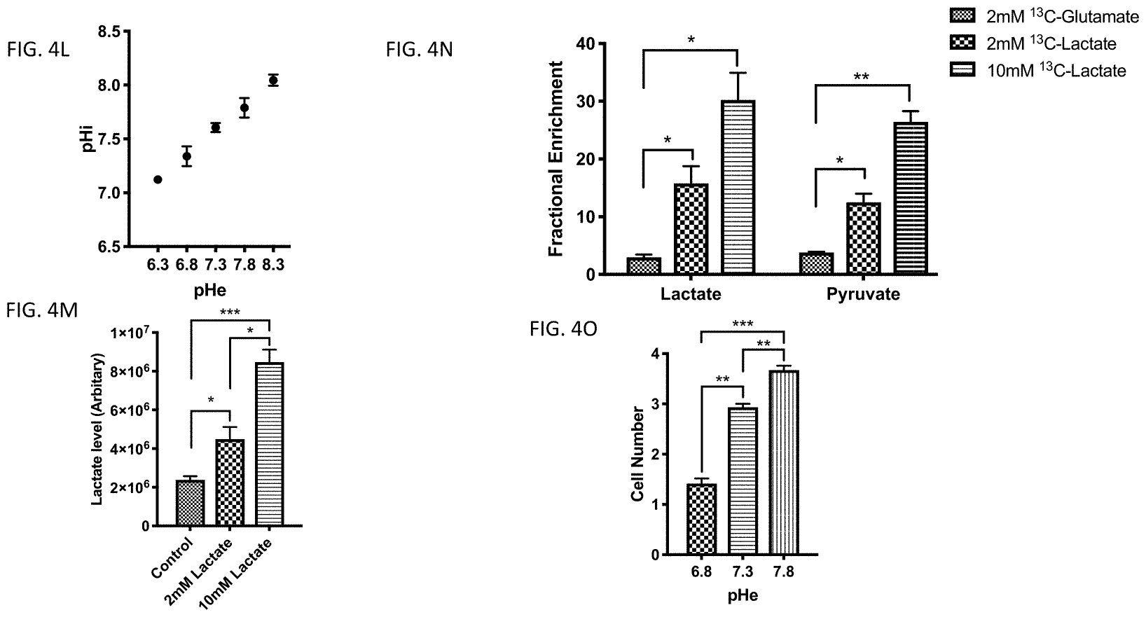

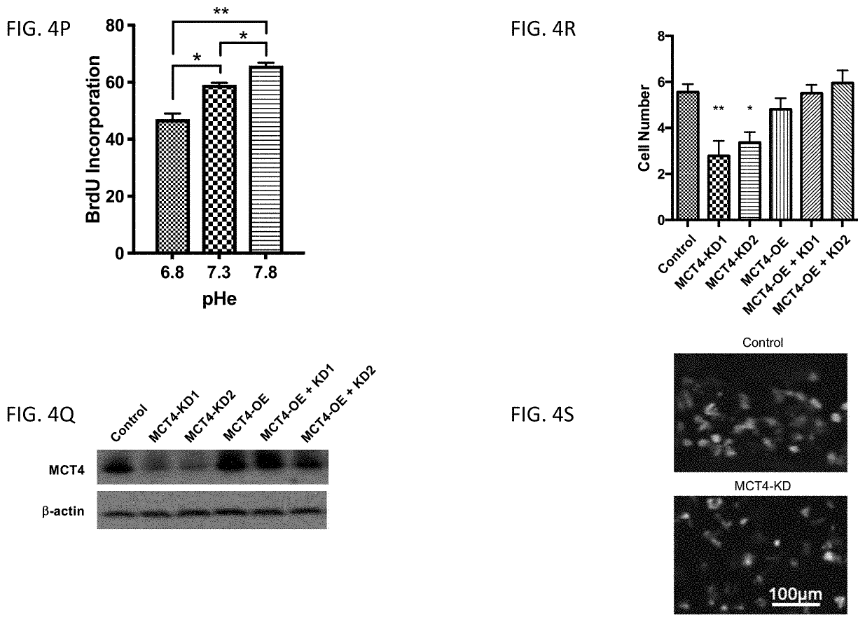

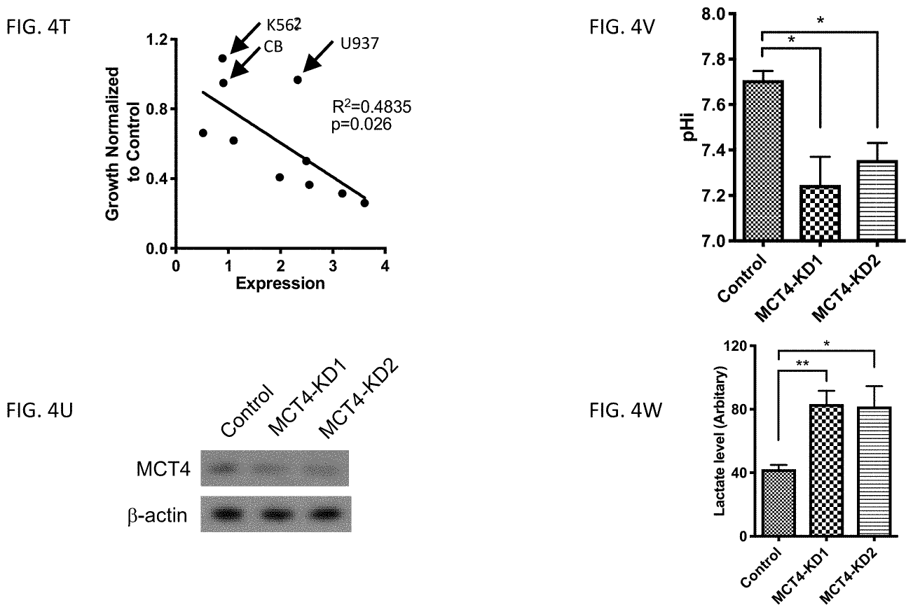

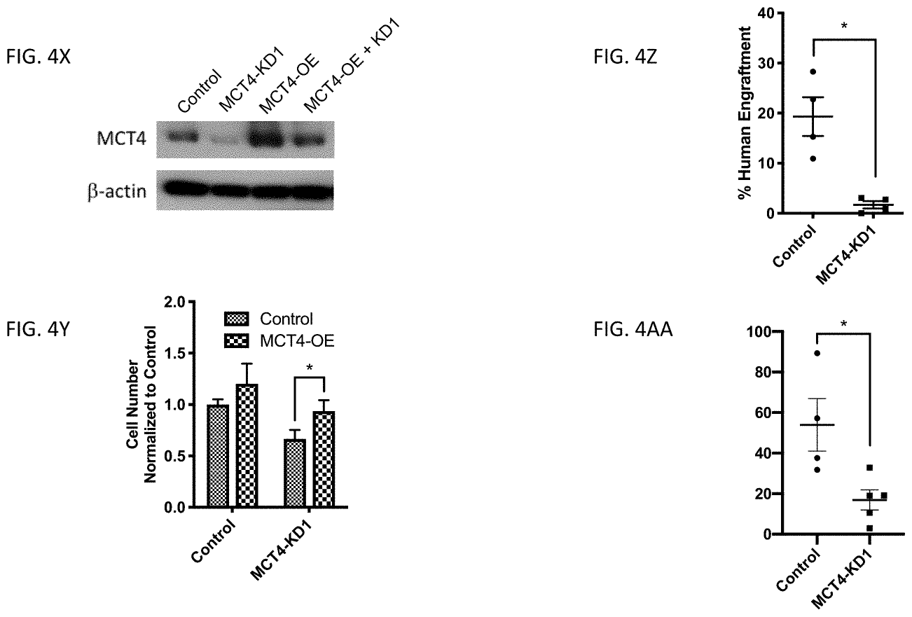

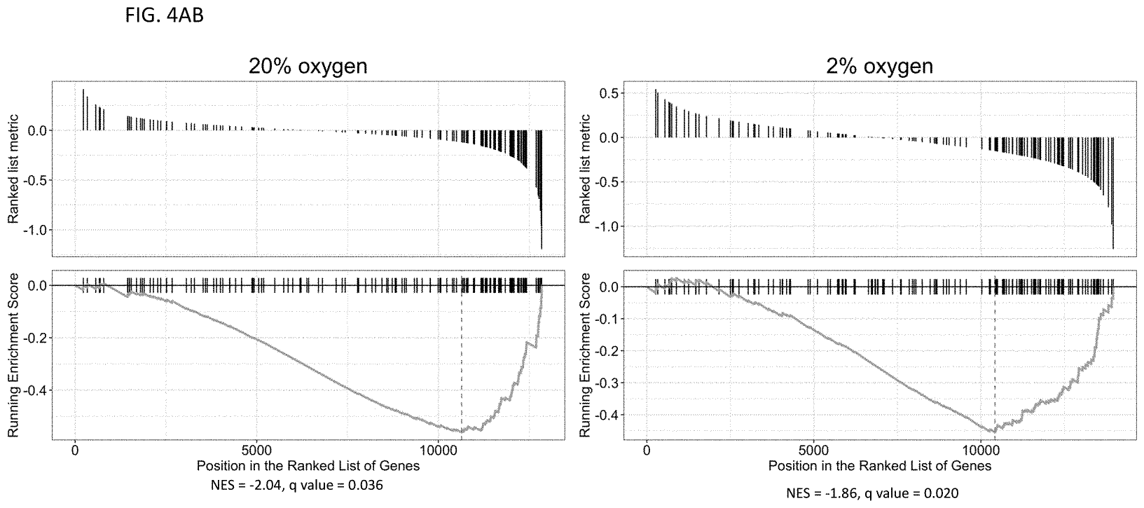

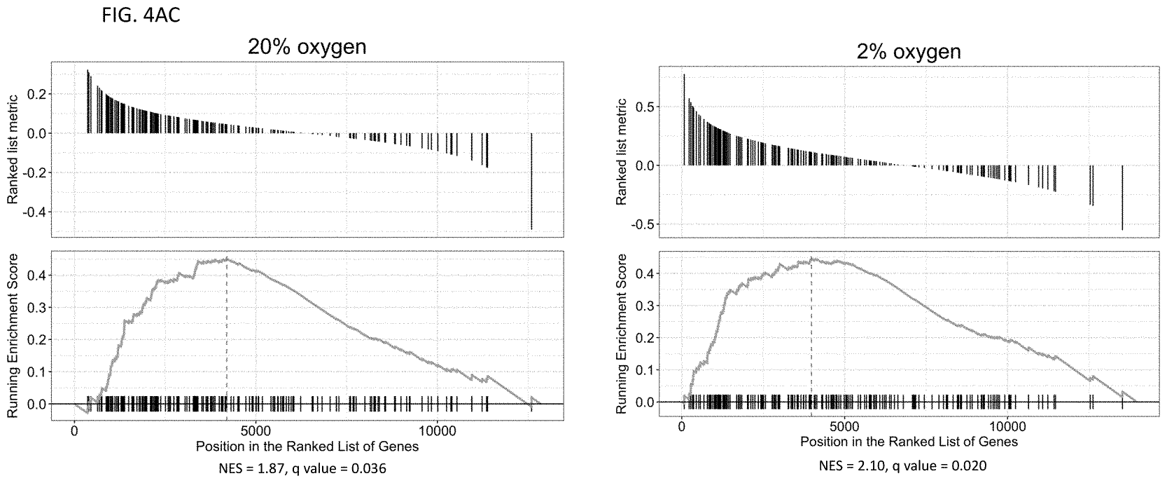

To examine the significance of MCT4 in AML, MCT4 was knocked out using CRISPR-Cas9 in MLL-AF9 murine AML, with non-targeting gRNA as negative controls. MCT4-KO was confirmed by Q-PCR (data not shown) and Western blot (FIG. 3A). MCT4-KO decreased pHi (from 7.6 to 7.3 and 7.5 to 7.0 in normoxic 20% and hypoxic 2% O₂ respectively) and increased intracellular lactate compared to controls (FIGS. 3B & 3C). Less extracellular lactate was found upon MCT4-KO (FIG. 4A). Changes in pH and lactate confirmed the functional importance of MCT4 and the lack of compensation of other FP/lactate regulators. MCT4-KO reduced the growth and clonogenic ability of MLL-AF9 AML in vitro (FIGS. 3D & FIG. 4B). pHi/lactate changes and growth inhibition were more significant in 2% O₂ compared to 20% O₂ (FIG. 4C-FIG. 4E). MCT4, as a downstream target of HIF-1α (Ullah et al., 2006), was induced in MLL-AF9 AML in 2% O₂ (FIG. 4F), accompanied by increased glucose uptake (FIGS. 4G & FIG. 4H) and extracellular lactate (FIG. 4A) suggesting that AML is more dependent on MCT4 in the low O₂ level (˜2%) that was previously found to exist in the bone marrow in vivo (Spencer et al., 2014).

In MLL-AF9 AML, loss of MCT4 modestly induced apoptosis (FIG. 3E) but significantly reduced proliferation (FIGS. 3F & FIG. 4I) and increased the cells in G1 (FIG. 4J). Reduction in CDK4, CDK6, Cyclin Dl and Cyclin B1 and increased cyclin-dependent kinase inhibitors p21 and p27 were found upon MCT4-KO (FIG. 3G). These findings suggest MCT4-KO in AML results in G1/S cell cycle arrest. Of note, MCT4-KO did not induce differentiation of AML, suggesting a dissociation of these two cell processes with altered proton handling (FIG. 4K).

MCT4-KO increased both H⁺ (low pH) and lactate. To define if increased H⁺ and/or lactate were responsible for growth inhibition, the inventors mimicked the changes of H⁺ and lactate by culturing MLL-AF9 AML in various pH and lactate levels. The pHi and intracellular lactate correlated with the extracellular pH and lactate (FIG. 4L-FIG. 4N). While reduction in extracellular pH suppressed AML growth (FIGS. 4O & FIG. 4P), increased extracellular lactate affected neither AML growth nor pHi (data not shown). These data suggest that the growth-inhibition resulting from loss of MCT4 is due to decreasing pHi rather than lactate accumulation.

To assess the in vivo effect of MCT4 suppression in AML, an inducible MCT4-shRNA was established in MLL-AF9 AML. MCT4-knockdown was confirmed by Q-PCR (data not shown) and Western blot (FIG. 4Q). Growth inhibition by MCT4-KD could be rescued by MCT4 overexpression (FIG. 4R), verifying the on-target effect of MCT4-KD.

MLL-AF9 AML cells carrying inducible MCT4- or scrambled-shRNA were transplanted into mice. Three weeks later, BM was sampled and AML engraftment was confirmed by FACS. shRNA was induced in vivo by doxycycline for 12 days. At day 2 of doxycycline treatment, acidification of pHi upon MCT4-KD was observed (FIGS. 3H & FIG. 4S). The median survival of MCT4-KD animals was extended (40-50 days) compared to scrambled shRNA control (28 days) (FIG. 3I). Notably, 6/17 (35%) mice with MCT4-KD survived beyond 60 days post doxycycline withdrawal. BM of surviving mice was harvested at that timepoint and no AML was detected in bone marrow (FIG. 3J). Secondary recipient mice transplanted with WBM from surviving mice were followed for 24 weeks without any sign of AML (FIG. 3J) demonstrating a functional loss of leukemia initiating cells (LIC). To verify this in vitro, MCT4-KD markedly inhibited serial replating capacity of cKit+MLL-AF9 (immunophenotypic LIC) cells (FIG. 3K). The on-target effect of MCT4-knockdown was confirmed with CRISPR-Cas9 MLL-AF9 in which in vivo MCT4-knockout by sgRNA resulted in reduction of AML cells (data not shown). To test whether the dependency of LIC on MCT4 was restricted to particular AML genotypes, the inventors performed knockdown experiments by introducing MCT4-shRNA into retrovirally-induced HoxA9-Meis1 and knock-in PML-RARα cells and demonstrated inhibition of leukemia initiation in vivo (FIG. 3L). Collectively, these data indicate that MCT4 inhibition reduces or eliminates LIC.

The inventors further examined the significance of MCT4 in human AML. MCT4-KD by shRNA suppressed the growth of all human AML cell lines but not other hematologic malignancies or cord blood (FIG. 4T). Expression of MCT4 correlated with the cytotoxicity upon MCT4-KD. Taking THP-1 as an exemplar, pHi reduction and lactate accumulation were observed upon MCT4-KD (FIG. 4U-FIG. 4W). Growth inhibition caused by MCT4-KD could be rescued by MCT4 overexpression (FIGS. 4X & FIG. 4Y). Constitutive MCT4-KD suppressed engraftment of THP-1 (FIG. 4Z) and MOLM-14 (FIG. 4AA) in vivo compared to scrambled shRNA control. However, MCT4-KD did not affect CML (K562) growth (FIG. 4T) or survival of transplanted mice (data not shown).

MCT4 was knocked down in primary human AML blasts (FIG. 3M) that decreased pHi and increased lactate (FIGS. 3N & 3O). Human AML blasts infected with either MCT4 or scrambled shRNA were transplanted into NSG mice. After 12-20 weeks, the level of human AML in the mouse BM revealed a significant reduction with MCT4-KD in 9/9 human AMLs carrying various cytogenetics and somatic mutations (summarized in Supplemental Table 1) (FIG. 3P). Knocking down MCT4 also reduced the LIC activity in primary human AML samples (CD34⁺CD38⁻) (FIG. 3Q). The patient-derived-xenograft model suggested that MCT4 inhibition reduced human AML LIC in vivo.

RNA sequencing was performed to compare the transcriptional signatures between non-targeting gRNA control and MCT4-KO MLL-AF9 AML in 20% and 2% 02. Replicates of samples formed well-separated clusters in the PCA plot, indicating good reproducibility of data and distinct gene expression under different conditions (data not shown). As expected, cells cultured in 2% O₂ significantly overexpressed hypoxia gene signatures compared to those grown in 20% O₂ (data not shown). MCT4-KO samples had decreased expression of both hypoxia and E2F related pathways (FIGS. 4AB & FIG. 4AC) further demonstrating that metabolism and proliferation were different between control and MCT4-KO cells.

Upregulation of MCT4 is Essential in AML-Adapted Glucose Metabolism

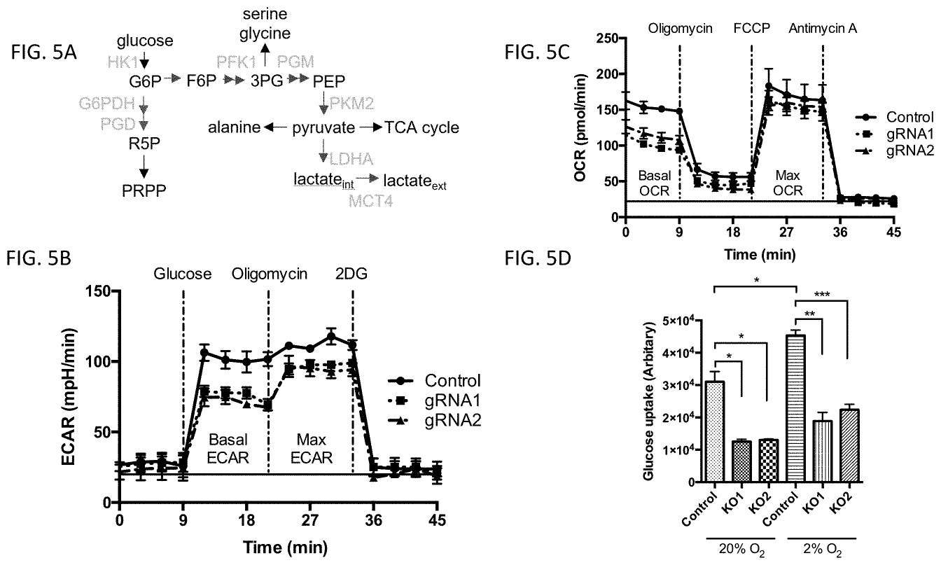

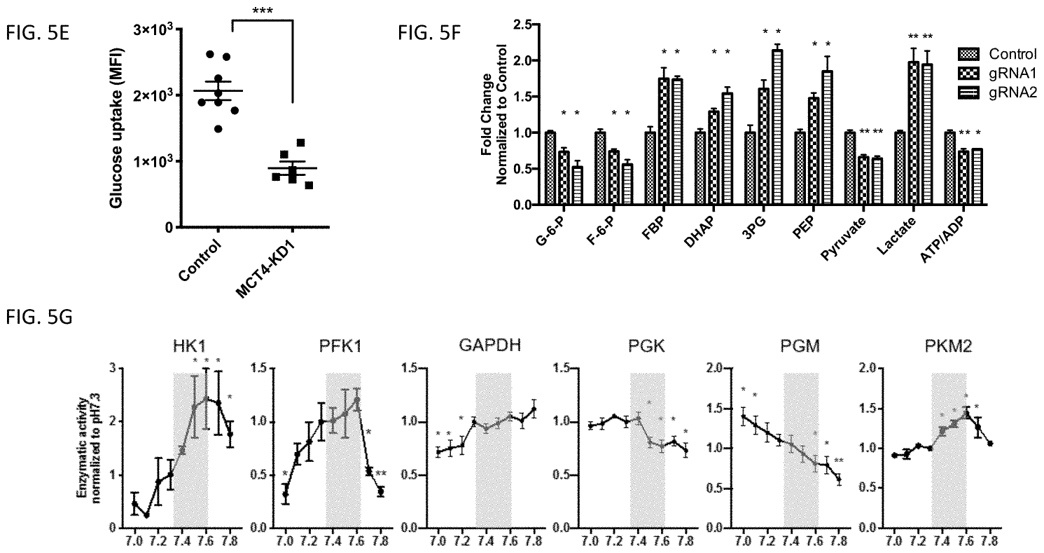

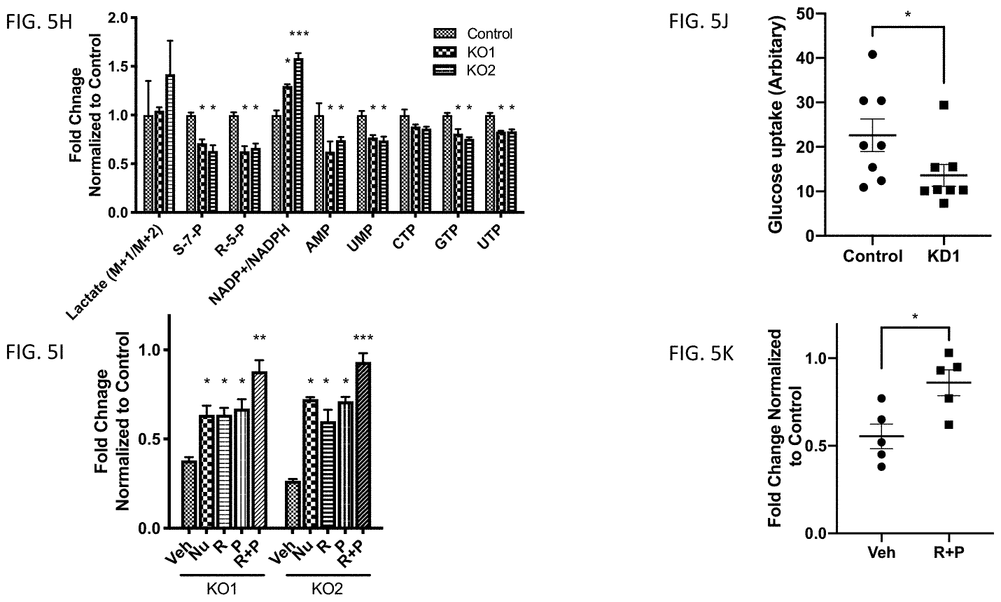

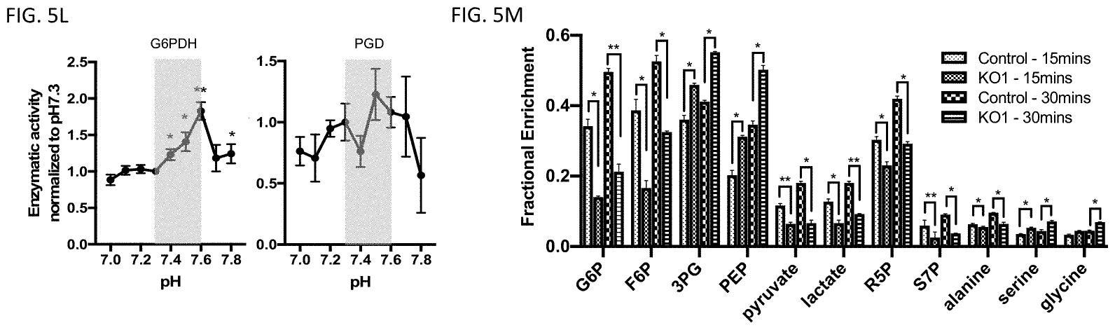

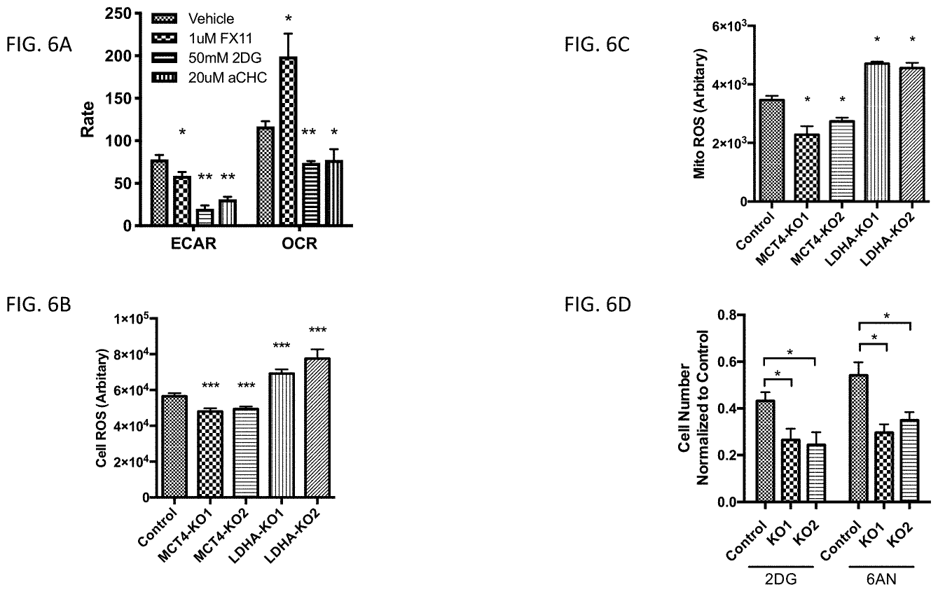

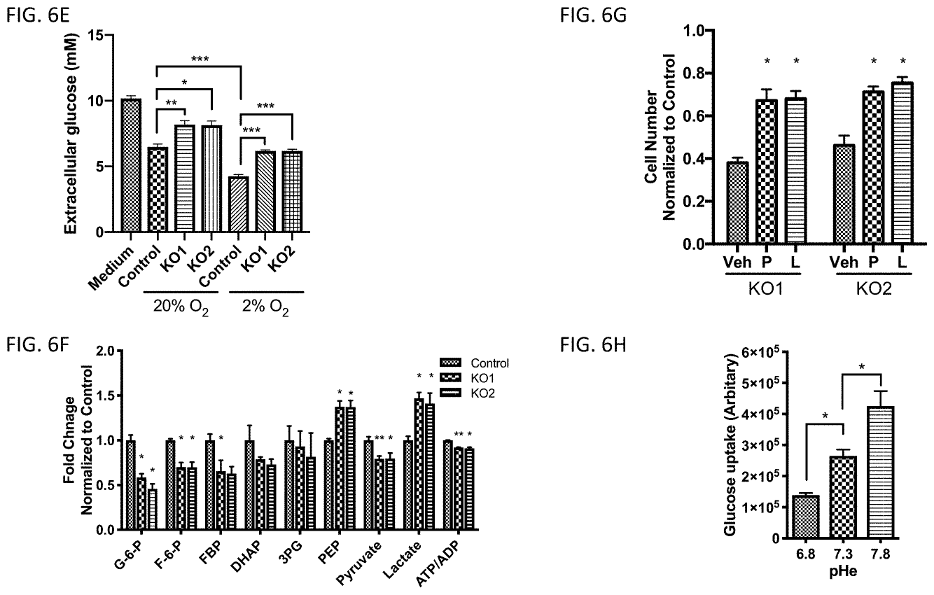

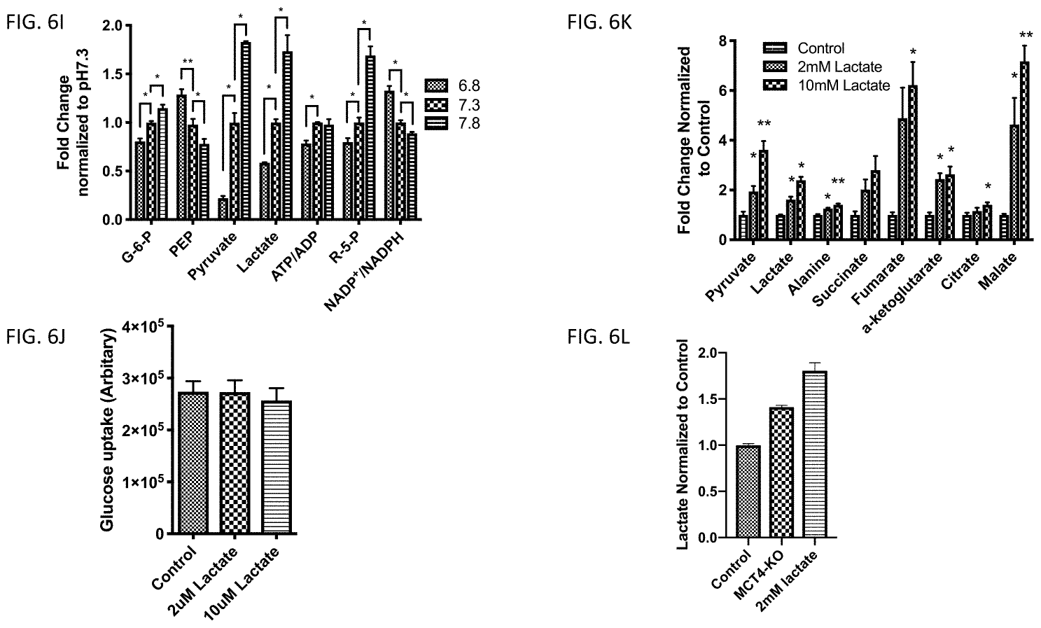

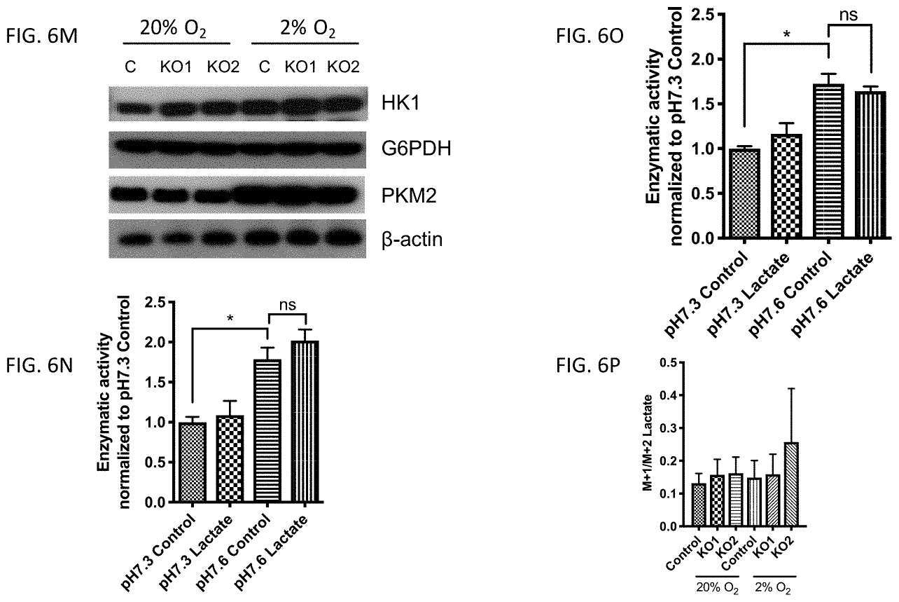

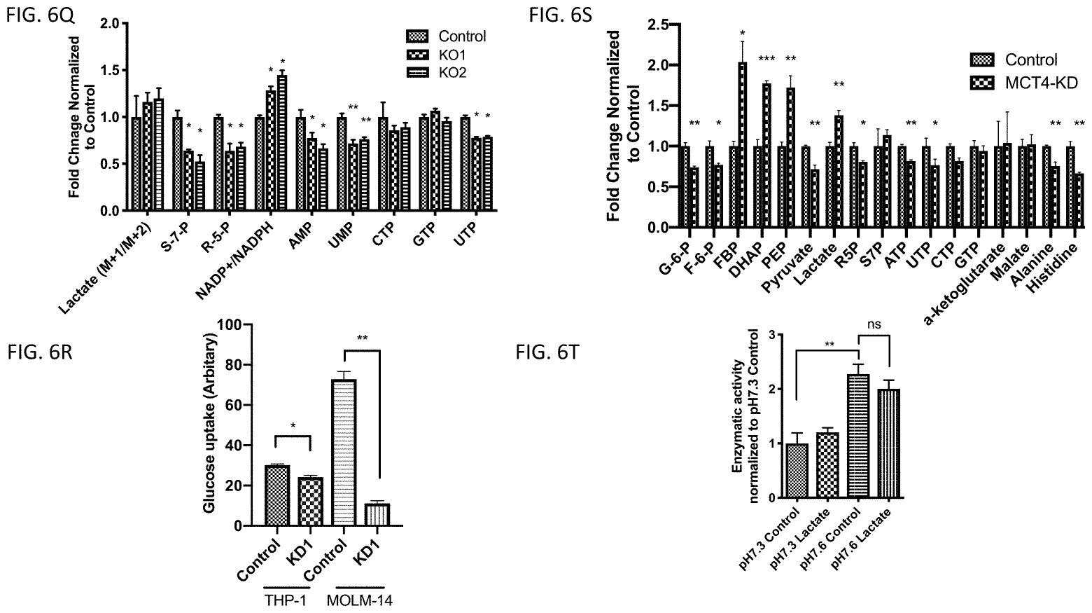

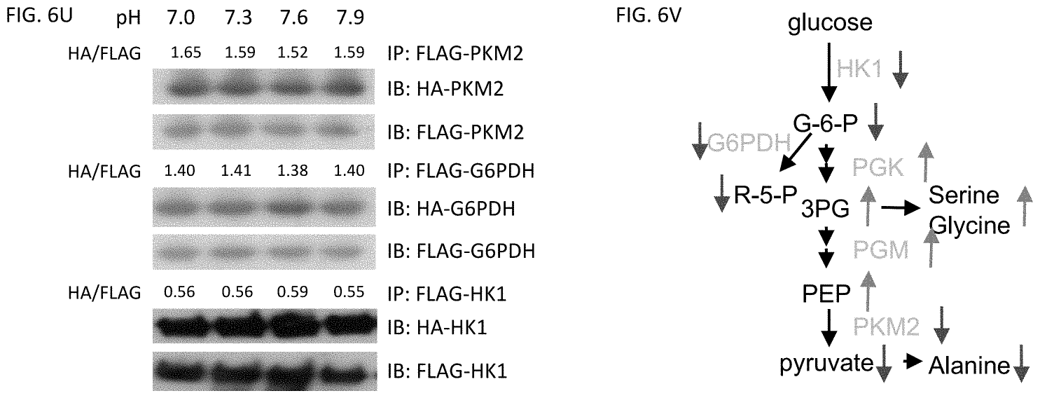

MCT4 exports the terminal glycolytic metabolites, H⁺ and lactate (FIG. 5A). It was previously shown that the enzyme for lactate generation, LDHA, impacts leukemic cell function (Wang et al., 2014) and the inventors anticipated that lactate production (changed by LDHA inhibition) and lactate export (changed by MCT4 inhibition) might have similar metabolic consequences. Pharmacologically inhibiting LDHA by FX11 in MLL-AF9 AML decreased extracellular acidification rate (ECAR) and increased oxygen consumption rate (OCR) (FIG. 6A). MCT4-KO, unlike LDHA inhibition, suppressed both ECAR (50%) and OCR (˜25%) (FIGS. 5B & 5C). It was previously reported that LDHA loss in AML enhances mitochondrial respiration and generates more ROS (Wang et al., 2014). In this study, LDHA-KO by CRISPR-Cas9 also increased ROS in AML, however, MCT4-KO reduced ROS unexpectedly (FIGS. 6B & 6C). Therefore, MCT4 affects glycolysis (ECAR) and mitochondrial respiration (OCR, ROS) differently from LDHA. Rather, the effect of MCT4-KO on ECAR and OCR is similar to that of 2-deoxyglucose (2DG) which globally suppresses glucose utilization (FIG. 6A). Other data also pointed toward MCT4 modulation of glucose metabolism. AML was more sensitive to MCT4-KO in 2% O₂ (FIG. 3D) when glucose uptake was greater (FIG. 4G). Also, MCT4-KO increased the effect of pharmacologic inhibitors of hexokinase1 (HK1) and glucose-6-phosphate dehydrogenase (G6PDH) by 2DG and 6-aminonicotinamide (6AN) respectively (FIG. 6D). Collectively, these results suggest that MCT4-KO affects glucose metabolism in addition to its role in H⁺/lactate transport.

The inventors noted that MCT4 reduction inhibited glucose uptake (FIGS. 5D, 5E & 6E). Analysis of intracellular glycolytic metabolites showed that loss of MCT4 reduced pyruvate and glucose-6-phosphate (G-6-P) (FIGS. 5F & 6F) but led to accumulation of phosphoenolpyruvate (PEP) and 3-phosphoglycerate (3-PG) and decreased ATP:ADP. Adding pyruvate to MCT4-KO cells increased flux into the TCA cycle and restored intracellular pyruvate levels (data not shown), but only partially rescued growth inhibition (FIG. 6G).

To test whether acidic pH or lactate accumulation led to the observed changes, the inventors compared the metabolite profile of MLL-AF9 AML cultured in different pH and lactate levels. Cells cultured in acidic pH exhibited reduced glucose uptake, ATP:ADP, and a similar metabolite profile as MCT4-KO (FIGS. 6H& 6I). Increased lactate did not affect glucose uptake, but did increase intracellular pyruvate and TCA metabolites (FIGS. 6J & 6K). The inventors confirmed that 2 mM lactate in culture increased intracellular lactate to a comparable level as knocking out MCT4 in MLL-AF9 AML (˜1.4-1.7 fold higher than non-targeting gRNA control) (FIG. 6L). The results suggested that the metabolic remodeling of MCT4-KO was primarily due to pH drop rather than lactate accumulation.

The inventors next sought to define the alterations in key enzymes that accounted for the metabolic changes of MCT4 inhibition. Phosphofructose kinase PFK1 (catalyzing fructose-6-phosphate (F-6-P)+ATP to fructose-1,6-bisphosphate (FBP)+ADP) is less active in acidic pH (Andres et al., 1990). However, MCT4-KO decreased F-6-P, increased FBP and decreased ATP:ADP (FIG. 5F) arguing against PFK1 inhibition.

HK1 is the first enzyme in glycolysis catalyzing glucose to G-6-P. PKM2 catalyzes ADP+PEP to ATP+pyruvate. Having shown reduction in G-6-P, pyruvate and ATP:ADP with accumulation of PEP, the inventors hypothesized that acidic pH following MCT4 loss may decrease the activity of HK1 and PKM2, inhibiting glycolysis and contributing to growth suppression. Expression of HK1 and PKM2 did not change upon MCT4-KO (FIG. 6M). The inventors therefore examined the effect of pH on enzymatic activity. HK1, PFK1 or PKM2 were expressed, purified and assayed in vitro for enzymatic activity at various pH. HK1 and PKM2 were most active at pH7.6 (as found in AML), exhibiting 2.4- and 1.4-fold increases respectively in activity compared to pH7.3 (as found in normal HSPC) (FIG. 5G). In contrast, the activity of PFK1 was stable between pH7.1-7.6. Adding lactate did not inhibit HK1 (FIG. 6N) and PKM2 (FIG. 6O). Therefore, HK1 and PKM2 show reduced activity in the setting of lower pH upon MCT4-KO. However, not all glycolytic enzymes respond similarly as phosphoglycerate kinase (PGK) and phosphoglycerate mutase (PGM) were ˜60% less active at pH7.6 compared to pH7.3.

Metabolic profiling also suggested that loss of MCT4 altered the PPP. MLL-AF9 AML cells were cultured in glucose-1,2-¹³C₂ to define the relative activity of glycolysis and PPP. Singly labelled lactate (M+1) reflects glucose transiting via PPP while doubly-labelled lactate (M+2) reflects glycolysis. The ratio of M+1/M+2 lactate between MCT4-KO and non-targeting gRNA control was not significantly different (FIG. 6P). It suggested that both glycolysis and PPP were similarly suppressed.

In MCT4-KO MLL-AF9 AML, ribose-5-phosphate (R-5-P) and sedoheptulose-7-phosphate (S-7-P) were reduced about 40% (FIGS. 5H & 6Q). In the PPP, NADP⁺ is reduced to NADPH by G6PDH and 6-phosphogluconate dehydrogenase (PGD) which catalyzes conversion from G-6-P to R-5-P. Increases in the NADP⁺/NADPH ratio implied reduced activity of either G6PDH or PGD. R-5-P is the end product of PPP and provides a major substrate for 5-phosphorybosyl-1-pyrophosphate (PRPP) for nucleotide biosynthesis. About 40% reduction in AMP and UMP was observed in MCT4-KO AML. Growth inhibition by MCT4-KO was partially rescued by either nucleosides or ribose (FIG. 5I). Full rescue could be obtained by adding both ribose and pyruvate Similar effects on metabolic remodeling with MCT4 inhibition were also observed in the human AML cell lines, THP-1 and MOLM-14 (FIGS. 6R & 6S) and primary AML myeloblasts (FIG. 5J). Adding ribose and pyruvate also rescued the growth inhibitory effect of MCT4-KD in primary human AML in vitro (FIG. 5K). These data confirm that both the glycolysis and PPP increases observed in AML depend on MCT4 and that growth control of AML was largely driven by the combined impact on glycolysis and PPP.

The inventors next examined the effect of pH on the activities of key enzymes in the PPP, G6PDH and PGD. G6PDH activity at pH7.3 was ˜50% of that at pH7.6 (FIG. 5L). Addition of lactate did not affect the activity of G6PDH (FIG. 6T). In contrast, PGD activity was unaltered by pH (FIG. 5L). These data suggest that G6PDH activity decreases at lower pH following the loss of MCT4, reducing PPP flux and critical products necessary for cell growth.

HK1, PKM2 and G6PDH form dimers or tetramers with increased enzymatic activity. The inventors examined the effect of pH on the ability to form polymers. FLAG- and HA-tagged proteins were transiently expressed in HEK293T and immunoprecipitated at different pH. Alterations in pH from 7.0 to 7.9 did not affect the polymer formation (FLAG:HA) of HK1, PKM2 and G6PDH (FIG. 6U). Therefore, enzyme polymerization was not the basis for altered enzyme activity.

Short term carbon flux was measured in the presence and absence of MCT4 at 15 and 30 minutes post uniformly-labeled ¹³C-glucose administration (FIG. 5M). Knocking out MCT4 reduced the ¹³C enrichment in upper (G6P, F6P) and lower glycolytic (pyruvate, lactate) and PPP metabolites (R5P, S7P) respectively (FIG. 5M). This was in keeping with the changes in enzymatic activity of isolated HK1, PKM2 and G6PDH the inventors observed in response to pH (FIGS. 5H and 5L). Correspondingly, ¹³C enrichment increased in the intermediate metabolites, 3PG and PEP as expected with decreased flux in lower glycolysis. Furthermore, changes in ¹³C incorporation in the amino acids derived from glycolytic intermediates (e.g. 3PG to serine/glycine and pyruvate to alanine) were observed (FIG. 5M). These data confirm that the alterations in enzymatic activity associated with pH changes on isolated proteins are present in intact AML cells (FIG. 6V). Further, MCT4 modulates proton shifts that fundamentally govern carbon handling, shifting it toward anabolic intermediates that drive amino and nucleic acid generation.

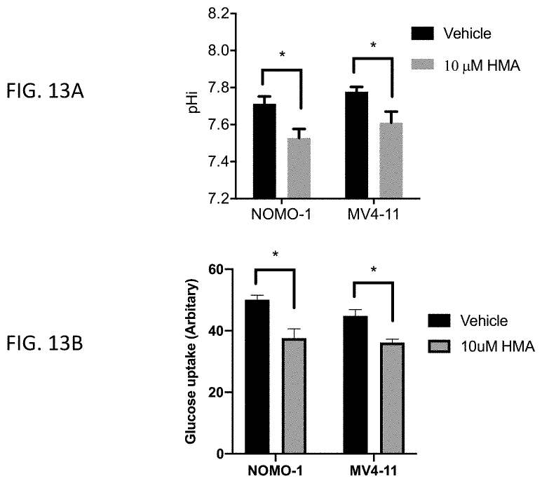

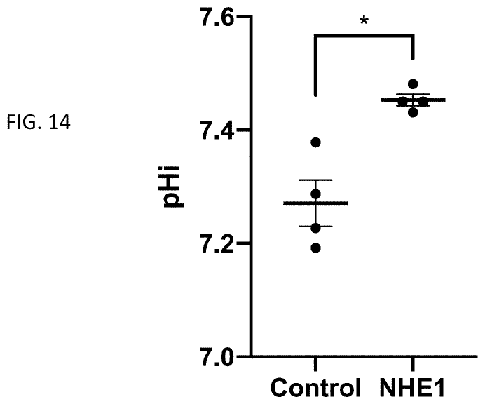

The inhibitory effect of intracellular acidification on glycolysis and pentose phosphate pathway is not restricted to MCT4. Pharmacologic suppression of Na⁺/H⁺ Exchanger 1 (NHE1) also resulted in intracellular acidification of FLT3^{ITD} AML cell lines (FIG. 13A). NHE1 inhibition suppressed the rate of glucose uptake (FIG. 13B). The anti-proliferative effect of NHE1 inhibition could be rescued by the supplement of ribose (R) and pyruvate (P). The data indicate that pHi regulating growth is not restricted to MCT4 but is a general property accompanying altered proton levels.

MCT4 Inhibition does not Affect Normal HSPC Function

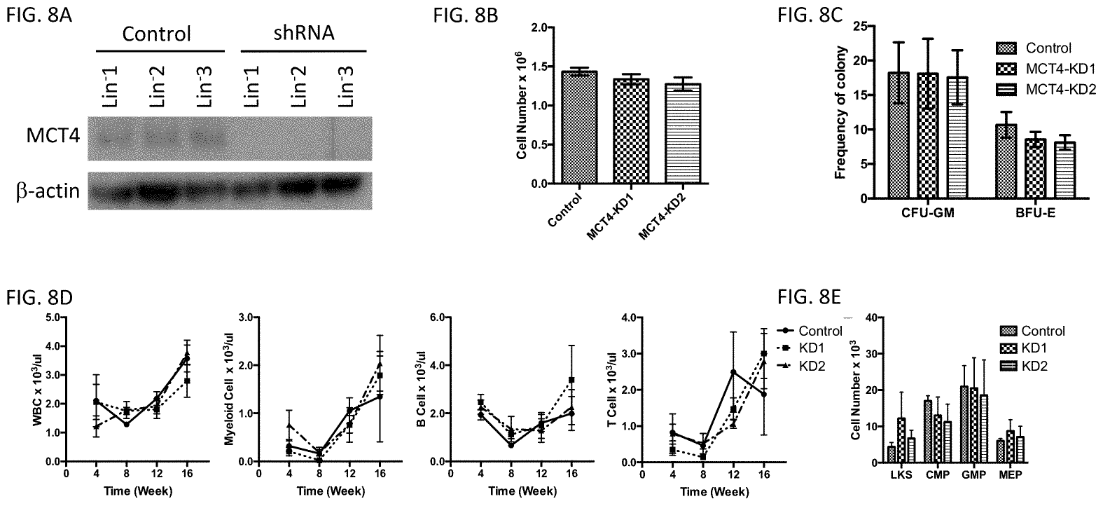

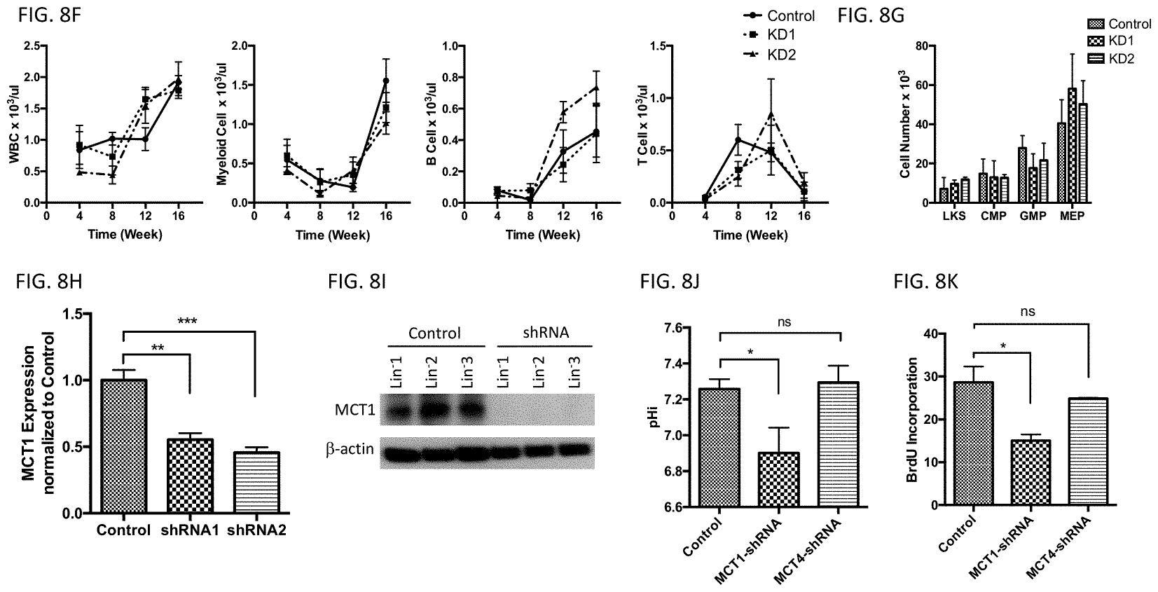

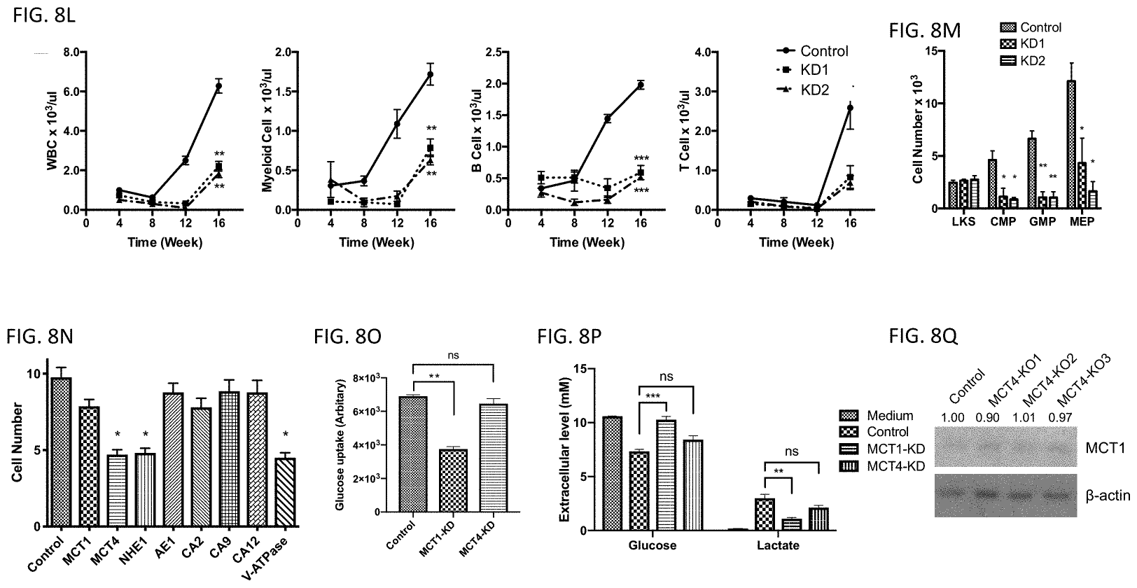

Normal mouse and human HSPC express lower levels of MCT4 compared to AML (FIG. 2E). To determine whether HSPC are dependent on MCT4, the inventors performed shRNA knockdown in mouse LKS cells. MCT4 was effectively reduced (FIG. 8A), however, MCT4-KD did not affect cellular growth or clonogenicity in vitro (FIGS. 8B & 8C).

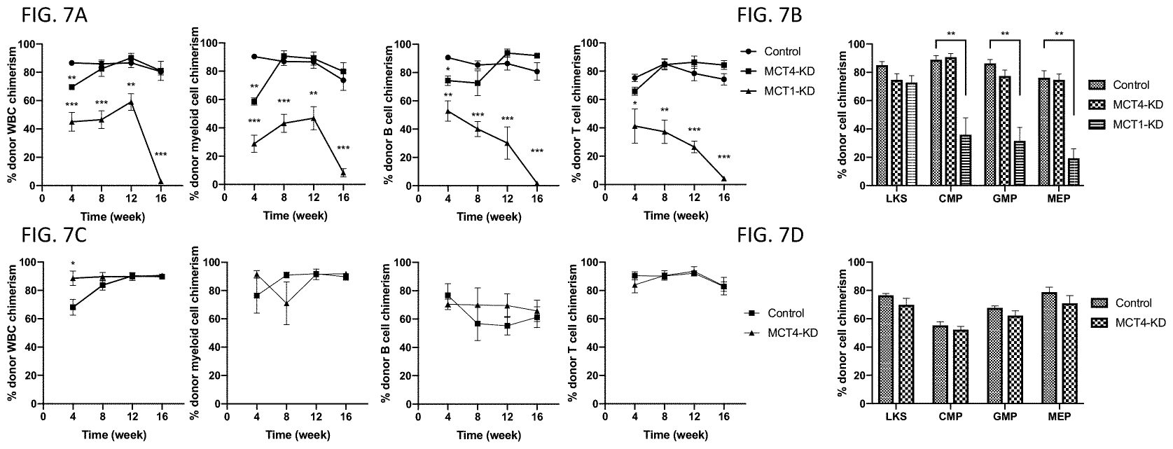

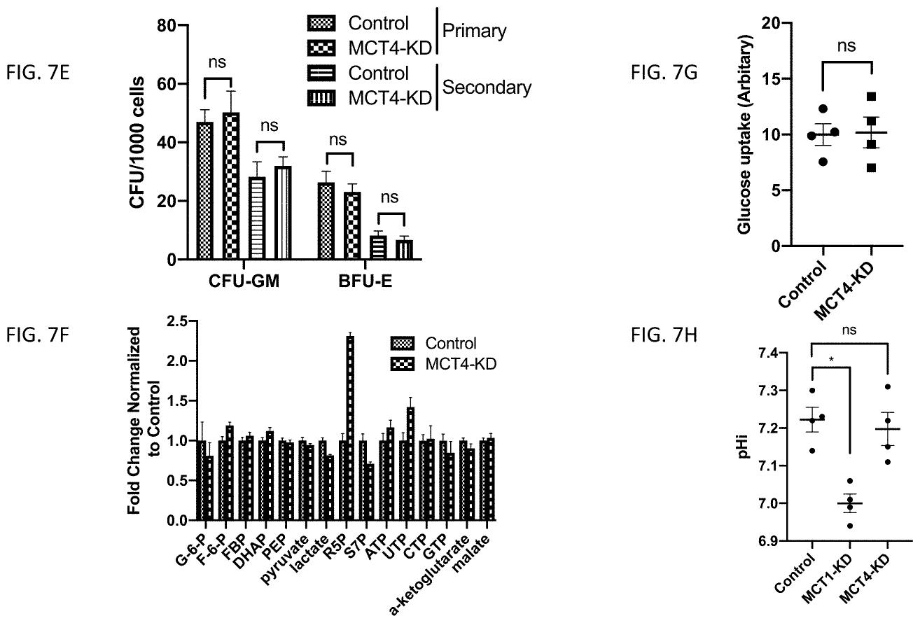

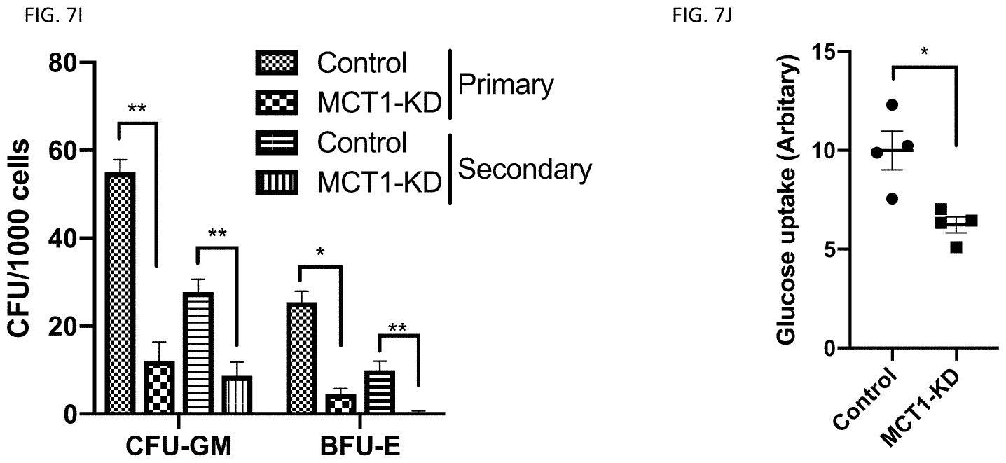

Testing HSPC function in vivo, the inventors transplanted the same number (20K) of LKS (CD45.1) transduced with MCT4- or scrambled shRNA in competition with 200K whole bone marrow CD45.2 cells into lethally irradiated CD45.2 recipient mice and traced the repopulation of donor cells for 16 weeks. MCT4-KD had minimal and only early effects on engraftment or multi-lineage differentiation of hematopoietic cells in vivo (FIGS. 7A & 8D). After 16 weeks, no significant differences between scrambled and MCT4-KD transduced cells were observed in various HSPC populations (FIGS. 7B & 8E). Secondary transplantation of MCT4-KD LKS was performed. No significant change in the differentiation or number of HSPC was apparent (FIGS. 7C, 7D, 8F & 8G). Furthermore, MCT4 knock down did not affect the intracellular pH, colony forming capacity or glucose metabolism of human cord blood samples (FIG. 7E-7H). Collectively, these data indicate that normal HSPC do not depend upon MCT4.

However, normal HSPC are functionally affected by loss of LDHA (Wang et al., 2014), suggesting that they are dependent on the production and perhaps levels of lactate. Since the expression of MCT1 and MCT4 in normal HSPC and AML is mutually exclusive (FIG. 2E), the inventors tested whether HSPC are more dependent on MCT1. MCT1 knockdown reduced pHi and proliferation of normal LKS in vitro (FIG. 8H-8K). In competitive transplant studies identical to that used for MCT4, LKS cells transduced with MCT1-KD shRNA had significantly reduced chimerism that declined with time and included CMP, GMP, MEP and the differentiated myeloid, B and T cells (FIGS. 7A, 7B, 8L & 8M) compared with LKS transduced with scrambled shRNA. By week 16 post-transplant, chimerism with the MCT1-KD cells was extinguished indicating exhaustion of HSC. MCT1-KD by shRNA also suppressed the intracellular pH, colony forming capacity and glucose uptake of human cord bloods (FIG. 7H-7J). In contrast, MCT1-KO only minimally reduced the growth of AML (FIG. 8N). The inventor further showed that MCT1-KD, but not MCT4-KD in normal GMP significantly reduced glucose uptake and extracellular lactate in vitro (FIGS. 8O & 8P). These findings indicate that proton shifting is not exclusively a dependency of malignant hematopoietic cells; normal cells also require it. However, normal HSPC and AML depend on different MCT family members and the extent of MCT activity is different as indicated by the differing pHi. The distinction in MCT usage suggests a potential therapeutic opportunity for targeting MCT4 in AML. Notably, MCT4 inhibition did not result in MCT1 upregulation (FIG. 8Q) indicating that compensation is not likely to offset MCT4 inhibitors in AML.

Epigenetic Regulation of MCT4 Expression by Histone Modification

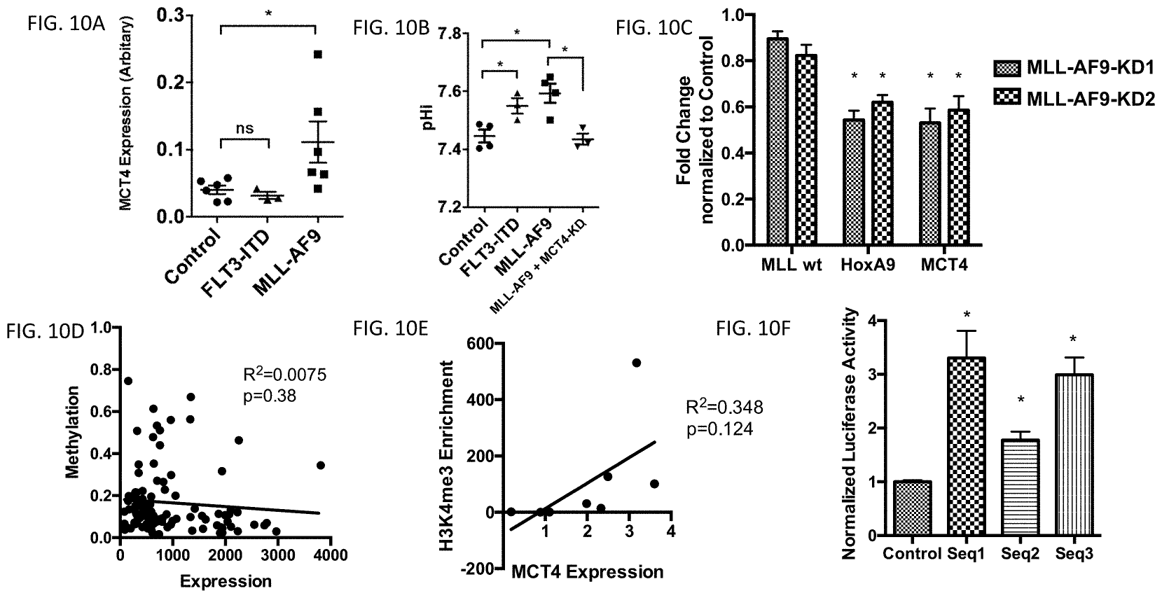

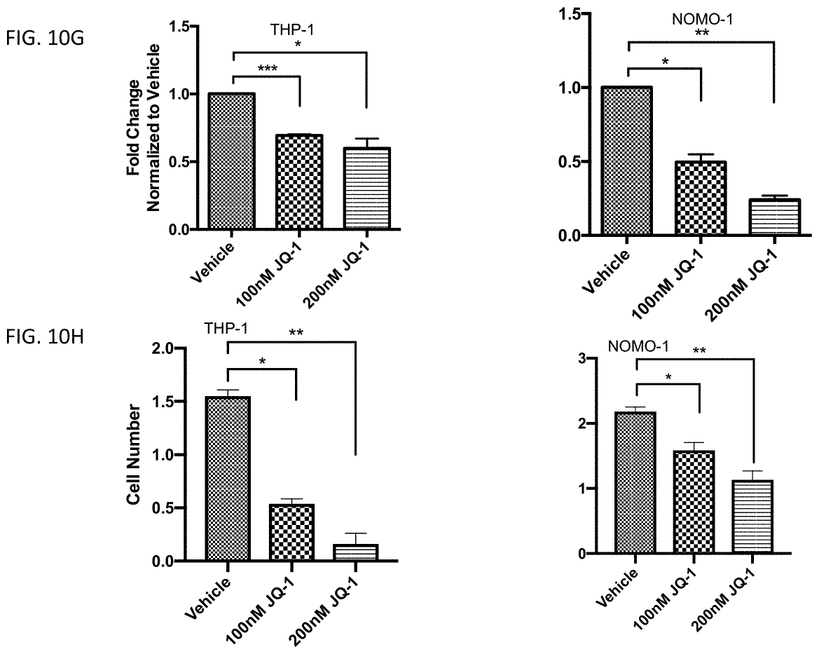

Induction of MLL-AF9 in normal mouse LKS significantly increased MCT4 expression and pHi at day 3 post-infection (FIGS. 10A & 10B). Knocking down MLL-AF9 reduced MCT4 expression (FIG. 10C) and pHi (FIG. 10B). Therefore, MLL-AF9 increases pHi by activating MCT4 expression. Since MCT4 promotor methylation does not correlate with MCT4 expression (FIG. 10D), MCT4 copy number variation is rare in AML (data not shown) and MLL-AF9 is a known epigenetic modifier (Devaiah et al., 2016; Nguyen et al., 2011), the inventors focused on epigenetic changes at the MCT4 locus.

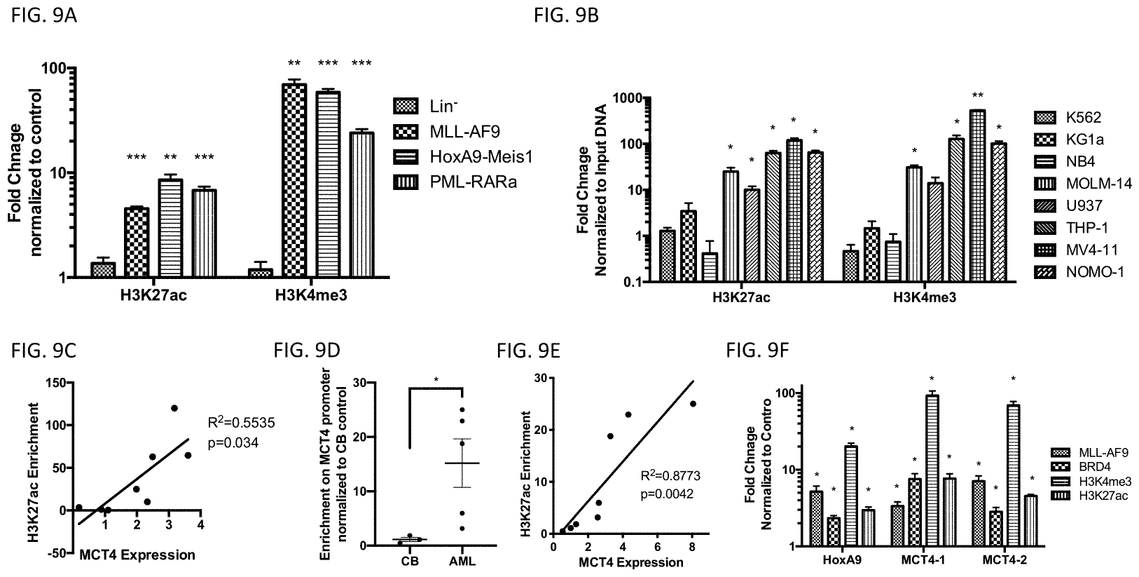

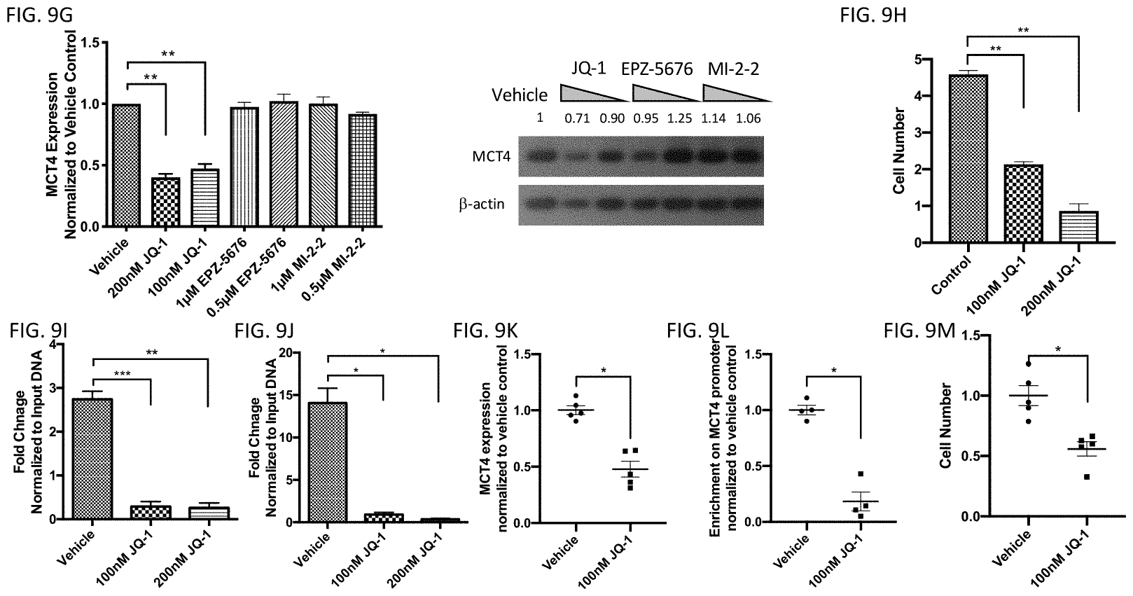

Epigenetic signatures on the MCT4 promoter across normal HSPC (Lin) and mouse AML cell lines were examined by ChIP-PCR. Transcriptional activation marks (H3K27ac and H3K4me3) were enriched on the MCT4 promoter in mouse AML but not normal HSPC (FIG. 9A) and corresponded with MCT4 expression (FIG. 1E). H3K27ac and H3K4me3 enrichment on the MCT4 promoter was also commonly found in human AML cell lines bearing a range of genetic abnormalities (Supplemental Table 2) (FIG. 9B). H3K27ac promoter marks correlated with MCT4 expression more than H3K4me3 marks (FIGS. 9C & 10E). Furthermore, H3K27ac enrichment on the MCT4 promoter was confirmed in primary human AML blasts (MNC) compared to normal CB control (CD34⁺) (FIG. 9D) and correlated with MCT4 expression (FIG. 9E).

Using a luciferase reporter, the inventors confirmed that MLL-AF9 directly activated the MCT4 promoter (FIG. 10F). ChIP for MLL1 and BRD4 demonstrated enrichment in the MCT4 promoter overlapping with H3K27ac and H3K4me3 in MLL-AF9 AML (FIG. 9F). Pharmacologic inhibition of the histone acetyltransferase, BRD4, by JQ-1 suppressed MCT4 expression. This was not seen with inhibition of DOT1L (by EPZ-5676) or MENIN (by MI-2-2), (FIG. 9G). JQ-1 treatment reduced the growth of MLL-AF9 AML in dose dependent manner (FIG. 9H). In human AML cell lines, THP-1 and NOMO-1, JQ-1 also suppressed the expression of MCT4 and growth (FIGS. 10G & 10H). BRD4 and H3K27ac enrichment on the MCT4 promoter was reduced by JQ-1 in mouse MLL-AF9 AML (FIGS. 9I & 9J). In primary human AML samples, JQ-1 had a similar effect on MCT4 expression, H3K27ac enrichment on the MCT4 promoter and growth as was observed in cell line models (FIG. 9K-9M). These data are consistent with MCT4 expression being driven by BRD dependent epigenetic modifications.

MCT4 Upregulation is Critical in Leukemogenesis

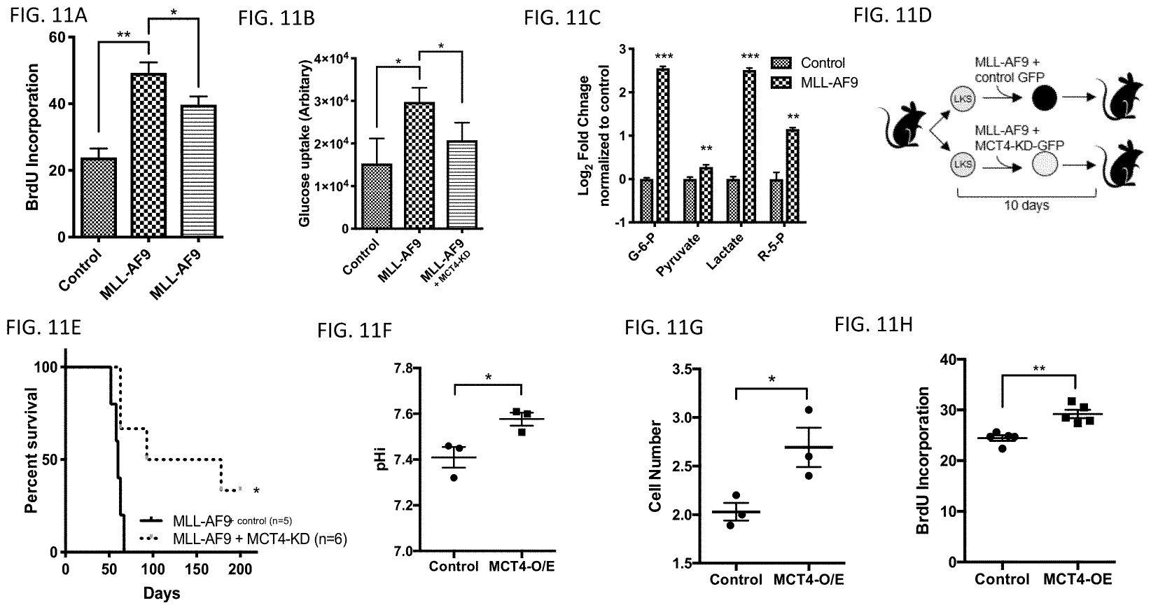

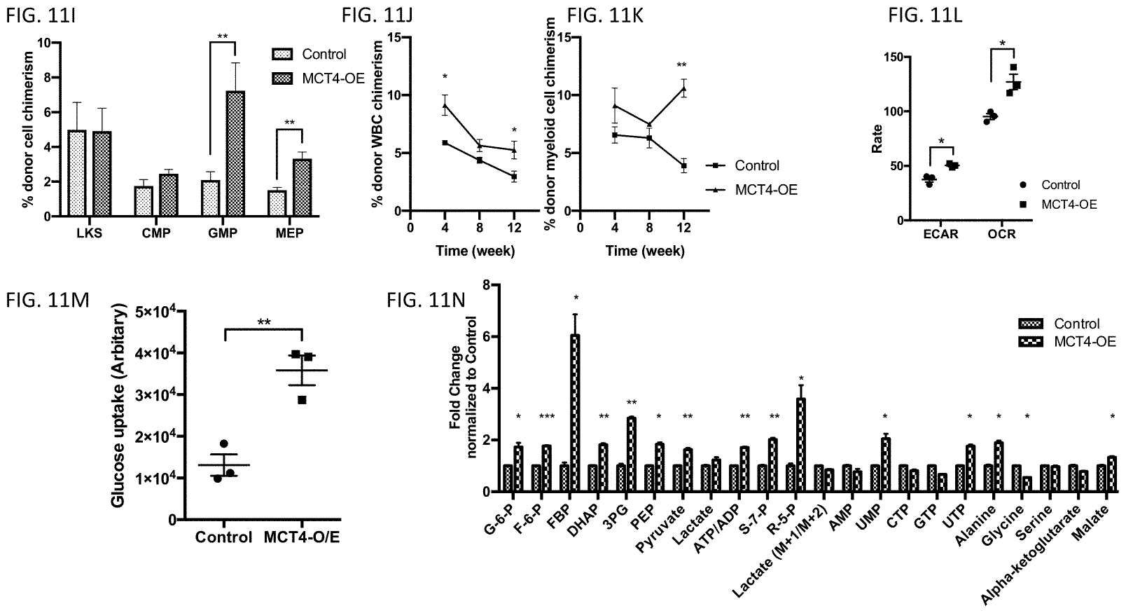

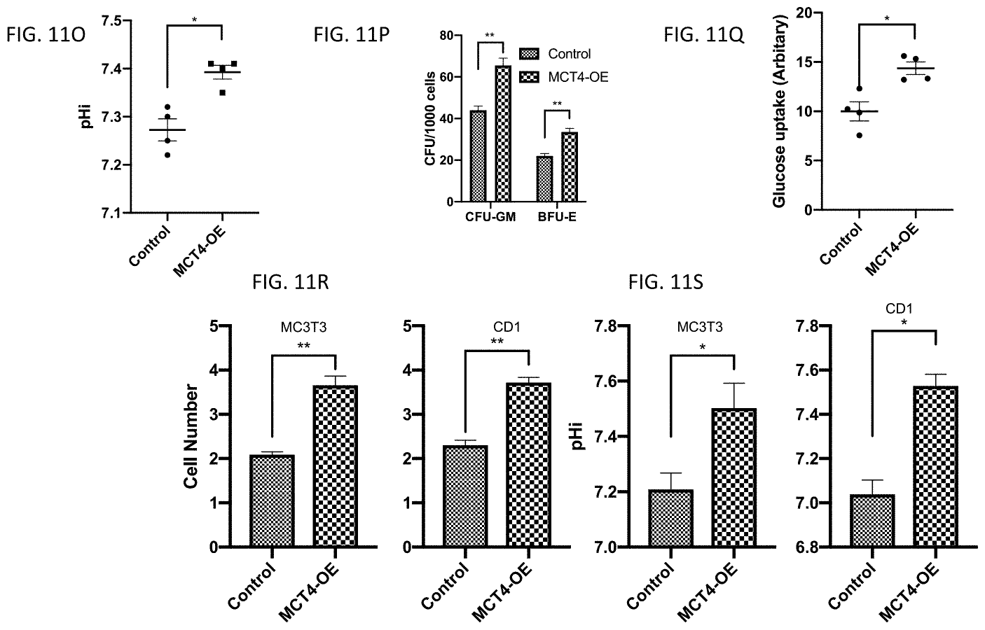

The inventors next tested whether MCT4 upregulation and intracellular alkalization are essential for the development of AML or just its maintenance. MLL-AF9 was transduced into normal GMP with either scrambled or MCT4 shRNA. The induced proliferation of preleukemic GMP by MLL-AF9 could be partially abrogated by MCT4-KD (FIG. 11A). Upon MLL-AF9 induction, GMP had increased glucose uptake and intracellular G-6-P, pyruvate, lactate and R-5-P (FIGS. 11B & 11C). Conversely, MCT4-KD suppressed glucose uptake in GMP.

Further, MLL-AF9 LKS cells were transduced with scrambled or MCT4 shRNA and transplanted into mice (FIG. 11D). MLL-AF9/MCT4-KD (135.5 days) showed a longer latency of disease development compared to MLL-AF9/scrambled shRNA control (60 days) (FIG. 11E). 2/6 (33.3%) mice in the MCT4-KD group did not develop leukemia and longer survival was observed, whereas all control mice died in 67 days. Collectively, these data indicate a direct role of MCT4 in the development of MLL-AF9 AML and point toward a competitive advantage for AML cells with intact MCT4.

MCT4 Overexpression Enhances Normal HSPC Growth

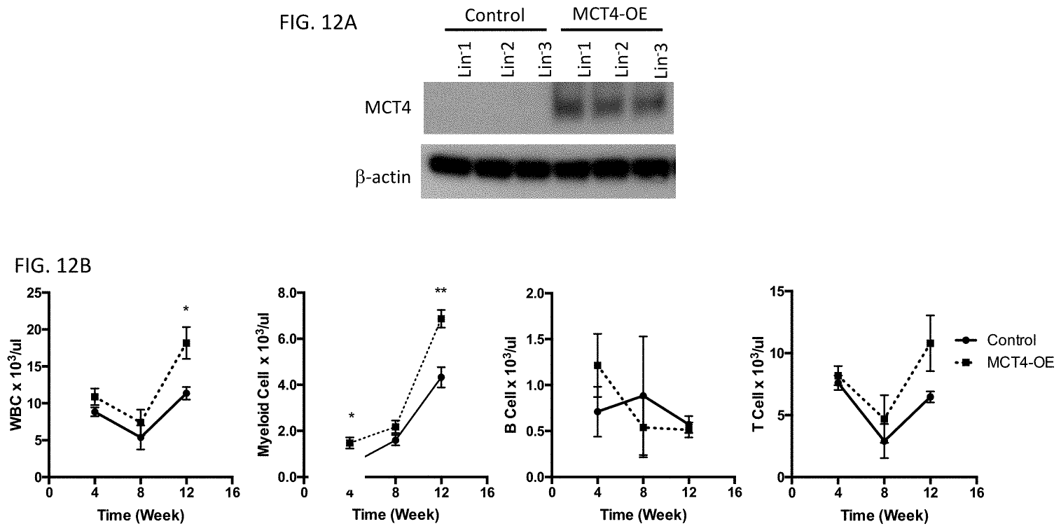

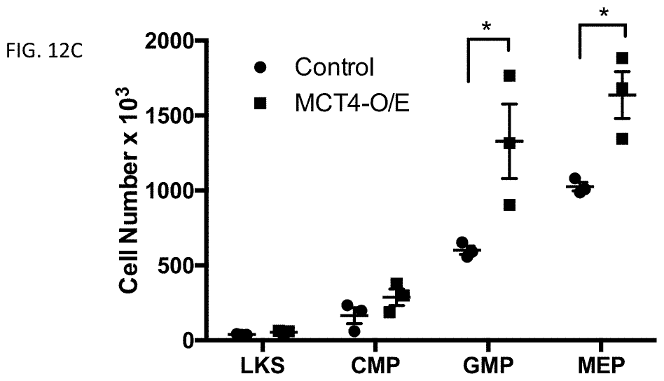

We tested whether proton shifting by MCT4 could affect normal HSPC by overexpressing MCT4 (MCT4-OE) in LKS cells (FIG. 12A). Intracellular alkalization and increased cell growth were observed upon MCT4-OE (FIG. 11F-11H). Transplantation of the MCT4-OE resulted in increased myeloid progenitors, GMP and MEP, in the bone marrow compared with empty vector control (FIG. 11I). Increased mature myeloid, but not B cells or T cells, was observed in the blood of the MCT4-OE group (FIGS. 11J, 11K, 12B & 12C). MCT4-OE in normal Lin⁻ BM cells increased ECAR and OCR (FIG. 11L), glucose uptake and intracellular levels of pyruvate, R-5-P and nucleotides (FIGS. 11M & 11N). Increases in ATP:ADP and decreased NADP⁺:NADPH ratios further supported the likelihood that MCT4-OE activates glycolysis and PPP. Similar growth proliferative effects of MCT4 were observed in human cord blood. Overexpressing MCT4 in cord blood increased the intracellular pH, colony forming capacity and glucose uptake in vitro (FIG. 11O-11Q).

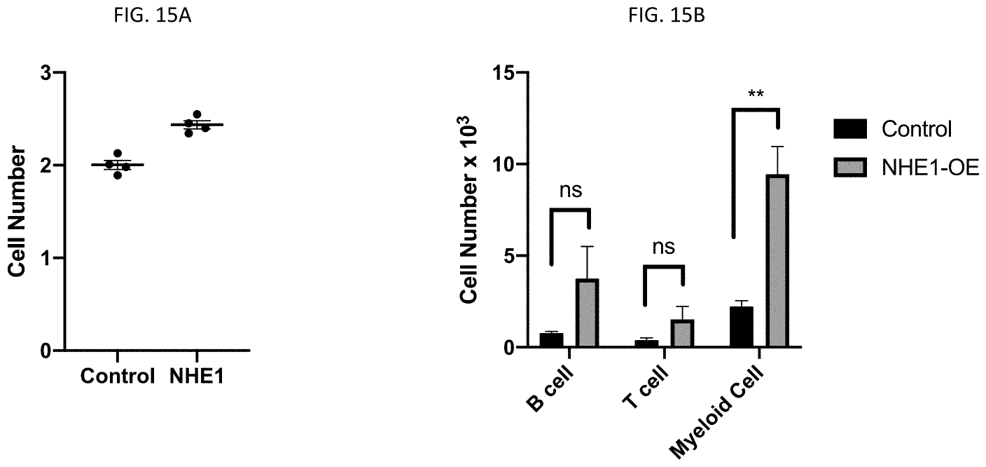

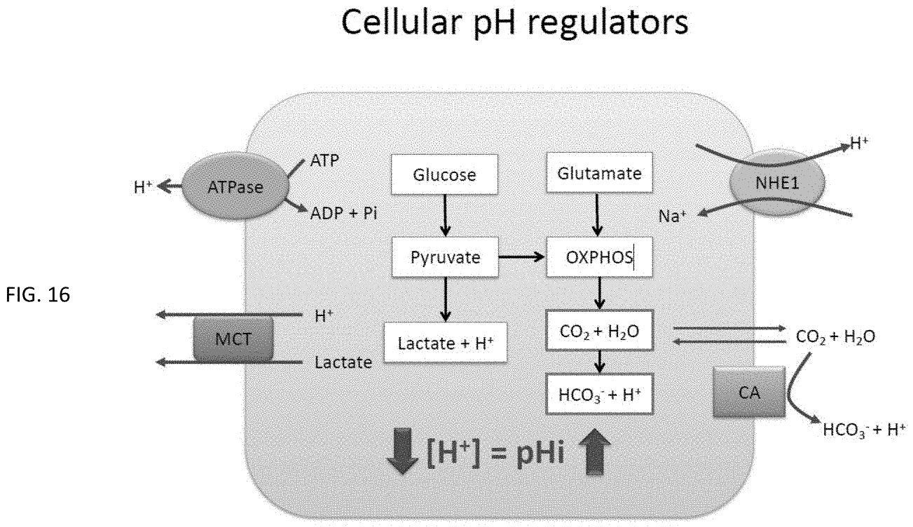

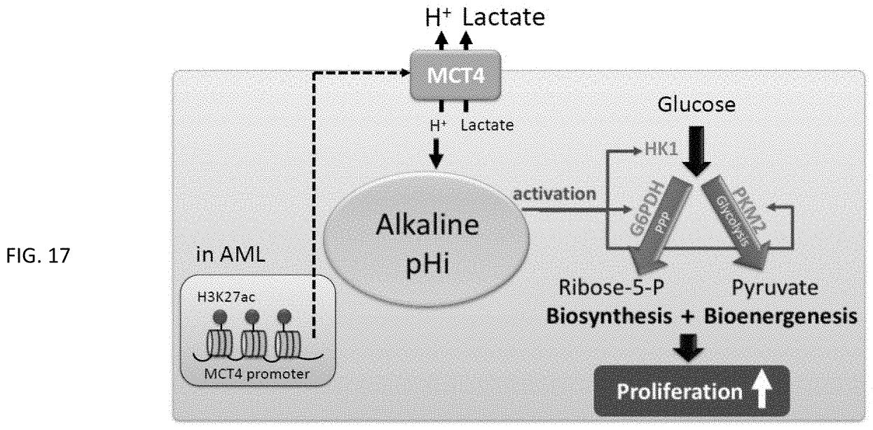

The growth enhancing effect of MCT4 was not only restricted to normal HSPC but also other normal cells. Overexpressing MCT4 in mouse pre-osteoblast (MC3T3) and CD-1 mouse primary tracheal and bronchial epithelial cells (CD1) resulted in intracellular alkalization and growth promotion (FIGS. 11R & 11S). Mouse embryonic fibroblast (MEF) cell did not respond to MCT4-OE (data not shown). These findings indicate that the growth enhancing effect of MCT4 are not restricted to myeloid cells but may be a more general biological effect across some, but not all, cell types. Using a different mechanism of intracellular alkalization, increased proliferation was also observed by overexpressing NHE1 in normal HSPC (FIGS. 15A-15B).

These results demonstrate that MCT4 overexpression is sufficient to enhance cell growth in association with a stereotypic alteration in carbon metabolism. While MCT4 is critical for the establishment of leukemia, it is not sufficient to render normal cells malignant, at least in the intervals studied. However, the alteration of cell growth by MCT4-induced proton shifting does not depend upon the presence of oncogenic mutations; it occurs in normal cells, particularly myeloid hematopoietic cells.

Discussion

This study demonstrates that intracellular alkalization via upregulation of MCT4 activity can remodel metabolism and induce cell growth. It is a process that does not depend upon complex ligand-receptor or signaling events and may therefore be a primitive growth regulatory mechanism.

The association of proton shifts with cell growth in plants is very distinctive from the mechanism proposed here. Both depend on differential activity of proteins based on the change in pH. However, in plants it is an active process induced by response to auxin. Auxin-induced pumping of protons into the cell wall space alters expansins and thereby the non-covalent interactions between cellulose microfibrils. Expansins are a family of low molecular weight (29-30 kDa) proteins abundant in many land plants within cell walls that, by mechanisms not well understood, change cellulose and hemicellulose interactions at low pH enabling cell wall elongation. This leads to irreversible cell expansion.

The process the inventors propose is that shuttling protons to the extracellular space secondarily increases pHi enhancing activity of key metabolic enzymes. This change in carbon handling and increased flux leads to the biomass generation needed for cell growth. MCT4 and other monocarboxylate transporters activity is primarily driven by a lactate gradient (Juel and Halestrap, 1999) providing a positive drive to cell growth through glycolysis itself. They are also HIF1α-responsive genes (Ullah et al., 2006) and may be induced through TLR activation of NF-kB pathways (Tan et al., 2015). All in keeping with our hypothesis that proton shifting may be a primitive means of enhancing growth in response to simple environmental cues.

The inventors focused on proton shifting based on leukemic cell dependency on it. Evidence for pHi regulation being abnormal in other settings of malignant cell growth include abnormal MCT1 in Ras-transformed cells (Le Floch et al., 2011) and renal cell carcinoma (Ambrosetti et al., 2018) and increased NHE1, a sodium-hydrogen antiporter, in breast cancer (Andersen et al., 2018). MCT1 inhibition has been shown to reduce glycolysis and tumor cell growth (Le Floch et al., 2011). MCT activity is proposed to allow for increased Warburg-like glycolytic activity by exporting the lactate by-product which would otherwise be inhibitory (Marchiq et al., 2015). Others have indicated that pHi may directly affect cell cycle regulators such as CDK1-cyclin B1 activity and account for cancer dependency (Putney and Barber, 2003). Also, mutations inducing histidine substitutions in regulatory proteins are sensitive to pHi by virtue of histidine's imidazole group being readily ionized and therefore available for ionic bond formation. Substitutions in TP53 Arg-His are common and can destabilize multimers of the protein (DiGiammarino et al., 2002). Multiple factors may therefore contribute to a selective advantage for cells with increased pHi in addition to the PPP and glycolysis flux changes that was found.

Altered MCT4 in malignancy may be governed by multiple factors. For example, altered MCT4 DNA methylation has been observed in renal cell carcinoma (Fisel et al., 2013). However, no correlation of MCT4 expression and DNA methylation in AML samples was found in the TCGA database nor were copy number variations in MCT4 evident. Rather, epigenetic marks with transcriptional activators H3K4me3 and H3K27ac were enriched on the MCT4 promoter region in AML. This was also evident upon reanalyzing the ChIP-seq data (GSE80779) previously published on human AML cell lines (data not shown) (Wan et al., 2017). The level of H3K27ac enrichment on the MCT4 promoter region significantly correlated with MCT4 expression in human AML. If altered epigenetic control is providing increased MCT4, then it is possible that mutations involving epigenetic modifiers, and not necessarily drivers of proliferation (e.g. kinases), may also gain a competitive advantage.

The combination of the hypoxia of the bone marrow that worsens with cell number (Spencer et al., 2014) and altered MCT4 epigenetic control provides a confluence of features in AML to augment MCT4. This may foster the dependency we observed on it and may account for the unexpected elimination of LIC in our xenograft transplantation experiments. Since elimination of LIC is paramount in achieving leukemic cure, the encouraging data argues for further consideration of MCT4 inhibition therapy. The differential dependency of normal and leukemic cells on MCT1 and MCT4 respectively and the lack of induction of MCT1 by MCT4 inhibition in AML further supports considering MCT4-targeted therapy in AML. Also, significant correlation of MCT4 expression and dependency on it was observed suggesting that MCT4 expression might serve as a biomarker for selecting the leukemias most likely to respond.

While the data shown in this example focuses only on the AML-cell intrinsic changes of MCT4 effects on other cells should be considered. Elevated extracellular lactate has been shown to suppress T/NK cell function and disable tumor immune surveillance (Brand et al., 2016). Whether decreased lactate export upon MCT4 suppression in AML restores the function of immune cells and helps eliminate AML is unclear and beyond the focus of this study, but would be an important topic in exploring therapeutic potential of MCT4 inhibition.

In sum, the data presented here raise the issue of proton shifts as central to growth regulation of at least some animal cells. Both normal and malignant hematopoietic cells were affected by MCT4 and with it, intracellular proton levels. Altering pHi changed the activity of key enzymes for energy and macromolecule generation resulting in cell proliferation. Proton shifting may be a mechanism by which cells can adjust growth kinetics rapidly and without dependence on complex signaling systems or ligand-receptor interactions. Whether this simple ion-driven process of growth control can be exploited to improve or impair cell growth in therapeutic settings is a topic these studies raise. Further, the potential to use cancer cells to unveil primitive cell growth regulators is supported by the data presented here.

### REFERENCES

Adijanto, J., and Philp, N.J. (2012). The SLC16A family of monocarboxylate transporters (MCTs)—physiology and function in cellular metabolism, pH homeostasis, and fluid transport. Curr Top Membr 70, 275-311.
Ambrosetti, D., Dufies, M., Dadone, B., Durand, M., Borchiellini, D., Amid, J., Pouyssegur, J., Rioux-Leclercq, N., Pages, G., Burel-Vandenbos, F., et al. (2018). The two glycolytic markers GLUT1 and MCT1 correlate with tumor grade and survival in clear-cell renal cell carcinoma. PLoS One 13, e0193477.
Andersen, A. P., Samsoe-Petersen, J., Oernbo, E. K., Boedtkjer, E., Moreira, J. M. A., Kveiborg, M., and Pedersen, S. F. (2018). The net acid extruders NHE1, NBCn1 and MCT4 promote mammary tumor growth through distinct but overlapping mechanisms. Int J Cancer 142, 2529-2542.
Andres, V., Carreras, J., and Cusso, R. (1990). Regulation of muscle phosphofructokinase by physiological concentrations of bisphosphorylated hexoses: effect of alkalinization. Biochem Biophys Res Commun 172, 328-334.
Bovenzi, C. D., Hamilton, J., Tassone, P., Johnson, J., Cognetti, D. M., Luginbuhl, A., Keane, W. M., Zhan, T., Tuluc, M., Bar-Ad, V., et al. (2015). Prognostic Indications of Elevated MCT4 and CD147 across Cancer Types: A Meta-Analysis. Biomed Res Int 2015, 242437.
Brand, A., Singer, K., Koehl, G. E., Kolitzus, M., Schoenhammer, G., Thiel, A., Matos, C., Bruss, C., Klobuch, S., Peter, K., et al. (2016). LDHA-Associated Lactic Acid Production Blunts Tumor Immunosurveillance by T and NK Cells. Cell Metab 24, 657-671.
Clasquin, M. F., Melamud, E., and Rabinowitz, J. D. (2012). LC-MS data processing with MAVEN: a metabolomic analysis and visualization engine. Curr Protoc Bioinformatics Chapter 14, Unit14 11.
Cole, A., Wang, Z., Coyaud, E., Voisin, V., Gronda, M., Jitkova, Y., Mattson, R., Hurren, R., Babovic, S., Maclean, N., et al. (2015). Inhibition of the Mitochondrial Protease ClpP as a Therapeutic Strategy for Human Acute Myeloid Leukemia. Cancer Cell 27, 864-876.
Devaiah, B. N., Case-Borden, C., Gegonne, A., Hsu, C. H., Chen, Q., Meerzaman, D., Dey, A., Ozato, K., and Singer, D. S. (2016). BRD4 is a histone acetyltransferase that evicts nucleosomes from chromatin. Nat Struct Mol Biol 23, 540-548.
DiGiammarino, E. L., Lee, A. S., Cadwell, C., Zhang, W., Bothner, B., Ribeiro, R. C., Zambetti, G., and Kriwacki, R. W. (2002). A novel mechanism of tumorigenesis involving pH-dependent destabilization of a mutant p53 tetramer. Nat Struct Biol 9, 12-16.
DiNardo, C. D., Stein, E. M., de Botton, S., Roboz, G. J., Altman, J. K., Mims, A. S., Swords, R., Collins, R. H., Mannis, G. N., Pollyea, D. A., et al. (2018). Durable Remissions with Ivosidenib in IDH1-Mutated Relapsed or Refractory AML. N Engl J Med 378, 2386-2398.
Dong, W., Keibler, M. A., and Stephanopoulos, G. (2017). Review of metabolic pathways activated in cancer cells as determined through isotopic labeling and network analysis. Metab Eng 43, 113-124.
Fisel, P., Kruck, S., Winter, S., Bedke, J., Hennenlotter, J., Nies, A. T., Scharpf, M., Fend, F., Stenzl, A., Schwab, M., et al. (2013). DNA methylation of the SLC16A3 promoter regulates expression of the human lactate transporter MCT4 in renal cancer with consequences for clinical outcome. Clin Cancer Res 19, 5170-5181.
German, N.J., Yoon, H., Yusuf, R. Z., Murphy, J. P., Finley, L. W., Laurent, G., Haas, W., Satterstrom, F. K., Guarnerio, J., Zaganjor, E., et al. (2016). PHD3 Loss in Cancer Enables Metabolic Reliance on Fatty Acid Oxidation via Deactivation of ACC2. Mol Cell 63, 1006-1020.
Juel, C., and Halestrap, A. P. (1999). Lactate transport in skeletal muscle—role and regulation of the monocarboxylate transporter. J Physiol 517 (Pt 3), 633-642.
Kim, D., Langmead, B., and Salzberg, S. L. (2015). HISAT: a fast spliced aligner with low memory requirements. Nat Methods 12, 357-360.
Kim, J., Chu, J., Shen, X., Wang, J., and Orkin, S. H. (2008). An extended transcriptional network for pluripotency of embryonic stem cells. Cell 132, 1049-1061.
Kirkman, H. N., and Gaetani, G. F. (1986). Regulation of glucose-6-phosphate dehydrogenase in human erythrocytes. J Biol Chem 261, 4033-4038.
Koivusalo, M., Welch, C., Hayashi, H., Scott, C. C., Kim, M., Alexander, T., Touret, N., Hahn, K. M., and Grinstein, S. (2010) Amiloride inhibits macropinocytosis by lowering submembranous pH and preventing Rac1 and Cdc42 signaling. J Cell Biol 188, 547-563.
Krivtsov, A. V., Twomey, D., Feng, Z., Stubbs, M. C., Wang, Y., Faber, J., Levine, J. E., Wang, J., Hahn, W. C., Gilliland, D. G., et al. (2006). Transformation from committed progenitor to leukaemia stem cell initiated by MLL-AF9. Nature 442, 818-822.
Le Floch, R., Chiche, J., Marchiq, I., Naiken, T., IIc, K., Murray, C. M., Critchlow, S. E., Roux, D., Simon, M. P., and Pouyssegur, J. (2011). CD147 subunit of lactate/H+ symporters MCT1 and hypoxia-inducible MCT4 is critical for energetics and growth of glycolytic tumors. Proc Natl Acad Sci USA 108, 16663-16668.
Lin, R., Elf, S., Shan, C., Kang, H. B., Ji, Q., Zhou, L., Hitosugi, T., Zhang, L., Zhang, S., Seo, J. H., et al. (2015). 6-Phosphogluconate dehydrogenase links oxidative PPP, lipogenesis and tumour growth by inhibiting LKB1-AMPK signalling. Nat Cell Biol 17, 1484-1496.
Lo Celso, C., Lin, C. P., and Scadden, D. T. (2011). In vivo imaging of transplanted hematopoietic stem and progenitor cells in mouse calvarium bone marrow. Nat Protoc 6, 1-14.
Love, M. I., Huber, W., and Anders, S. (2014). Moderated estimation of fold change and dispersion for RNA-seq data with DESeq2. Genome Biol 15, 550.
Lu, C., Ward, P. S., Kapoor, G. S., Rohle, D., Turcan, S., Abdel-Wahab, O., Edwards, C. R., Khanin, R., Figueroa, M. E., Melnick, A., et al. (2012). IDH mutation impairs histone demethylation and results in a block to cell differentiation. Nature 483, 474-478.
Man, C. H., Lam, S. S., Sun, M. K., Chow, H. C., Gill, H., Kwong, Y. L., and Leung, A. Y. (2014). A novel tescalcin-sodium/hydrogen exchange axis underlying sorafenib resistance in FLT3-ITD+ AML. Blood 123, 2530-2539.
Marchiq, I., Le Floch, R., Roux, D., Simon, M. P., and Pouyssegur, J. (2015). Genetic disruption of lactate/H+ symporters (MCTs) and their subunit CD147/BASIGIN sensitizes glycolytic tumor cells to phenformin. Cancer Res 75, 171-180.
Martinez-Nunez, R. T., and Sanford, J. R. (2016). Studying Isoform-Specific mRNA Recruitment to Polyribosomes with Frac-seq. Methods Mol Biol 1358, 99-108.
Nguyen, A. T., Taranova, O., He, J., and Zhang, Y. (2011). DOT1L, the H3K79 methyltransferase, is required for MLL-AF9-mediated leukemogenesis. Blood 117, 6912-6922.
Ossenkoppele, G., and Lowenberg, B. (2015). How I treat the older patient with acute myeloid leukemia. Blood 125, 767-774.
Park, J. O., Rubin, S. A., Xu, Y. F., Amador-Noguez, D., Fan, J., Shlomi, T., and Rabinowitz, J. D. (2016). Metabolite concentrations, fluxes and free energies imply efficient enzyme usage. Nat Chem Biol 12, 482-489.
Putney, L. K., and Barber, D. L. (2003). Na—H exchange-dependent increase in intracellular pH times G2/M entry and transition. J Biol Chem 278, 44645-44649.
Raffel, S., Falcone, M., Kneisel, N., Hansson, J., Wang, W., Lutz, C., Bullinger, L., Poschet, G., Nonnenmacher, Y., Barnert, A., et al. (2017). BCAT1 restricts alphaKG levels in AML stem cells leading to IDHmut-like DNA hypermethylation. Nature 551, 384-388.
Rayle, D. L., and Cleland, R. E. (1992). The Acid Growth Theory of auxin-induced cell elongation is alive and well. Plant Physiol 99, 1271-1274.
Stein, E. M., DiNardo, C. D., Pollyea, D. A., Fathi, A. T., Roboz, G. J., Altman, J. K., Stone, R. M., DeAngelo, D. J., Levine, R. L., Flinn, I. W., et al. (2017). Enasidenib in mutant IDH2 relapsed or refractory acute myeloid leukemia. Blood 130, 722-731.
Stirewalt, D. L., Meshinchi, S., Kopecky, K. J., Fan, W., Pogosova-Agadjanyan, E. L., Engel, J. H., Cronk, M. R., Dorcy, K. S., McQuary, A. R., Hockenbery, D., et al. (2008). Identification of genes with abnormal expression changes in acute myeloid leukemia. Genes Chromosomes Cancer 47, 8-20.
Sykes, D. B., Kfoury, Y. S., Mercier, F. E., Wawer, M. J., Law, J. M., Haynes, M. K., Lewis, T. A., Schajnovitz, A., Jain, E., Lee, D., et al. (2016). Inhibition of Dihydroorotate Dehydrogenase Overcomes Differentiation Blockade in Acute Myeloid Leukemia. Cell 167, 171-186 e115.
Tan, Z., Xie, N., Banerjee, S., Cui, H., Fu, M., Thannickal, V. J., and Liu, G. (2015). The monocarboxylate transporter 4 is required for glycolytic reprogramming and inflammatory response in macrophages. J Biol Chem 290, 46-55.
Tanner, L. B., Goglia, A. G., Wei, M. H., Sehgal, T., Parsons, L. R., Park, J. O., White, E., Toettcher, J. E., and Rabinowitz, J. D. (2018). Four Key Steps Control Glycolytic Flux in Mammalian Cells. Cell Syst 7, 49-62 e48.
TeSlaa, T., and Teitell, M. A. (2014). Techniques to monitor glycolysis. Methods Enzymol 542, 91-114.
Ullah, M. S., Davies, A. J., and Halestrap, A. P. (2006). The plasma membrane lactate transporter MCT4, but not MCT1, is up-regulated by hypoxia through a HIF-1alpha-dependent mechanism. J Biol Chem 281, 9030-9037.
Wang, Y. H., Israelsen, W. J., Lee, D., Yu, V. W. C., Jeanson, N. T., Clish, C. B., Cantley, L. C., Vander Heiden, M. G., and Scadden, D. T. (2014). Cell-state-specific metabolic dependency in hematopoiesis and leukemogenesis. Cell 158, 1309-1323.
Webb, B. A., Chimenti, M., Jacobson, M. P., and Barber, D. L. (2011). Dysregulated pH: a perfect storm for cancer progression. Nat Rev Cancer 11, 671-677.
White, K. A., Ruiz, D. G., Szpiech, Z. A., Strauli, N. B., Hernandez, R. D., Jacobson, M. P., and Barber, D. L. (2017). Cancer-associated arginine-to-histidine mutations confer a gain in pH sensing to mutant proteins. Sci Signal 10.

STAR Methods

Cell Processing

Primary human AML samples from patients were provided by Prof. Anskar Y. H. Leung (The University of Hong Kong). The study was approved by the Institutional Review Board (IRB; reference no: UW05-183) at Queen Mary Hospital, Hong Kong in accordance with the Declaration of Helsinki. The clinical information was summarized in Supplemental Table 1. Mononuclear cells (MNC) from blood and/or BM of AML patients were purified using Ficoll-Paque™ Plus (Amersham Biosciences) and stored in liquid nitrogen until use. Cord blood samples were obtained from the Pasquarello Tissue Bank—CMCF, Dana Farber Cancer Institute (IRB #2010P0002371). CD34⁺ cells were purified by EasySep™ Human Cord Blood CD34 Positive Selection Kit II (Stemcell Technologies) and stored in liquid nitrogen until use.

Cell Culture

Human AML cell lines (K562, KG1, ML2, Kasumi-1, MOLM-13, MOLM-14, MV4-11, THP-1, MONO-MACE, HL-60, NB4, U937 and NOMO-1) were purchased from the American Type Culture Collection (ATCC) and cultured in RPMI-1640 media supplemented with 10% FBS and 1% penicillin/streptomycin (P/S).

Retroviral transduction model of MLL-AF9 or HoxA9-Meis1 AML were generated by infecting normal mouse GMP (Lin⁻Kit⁺Sca-1⁻CD34⁺CD16/32⁺) (FASC antibodies summarized in Supplemental Table 4) by MSCV constructs. Infected cells were injected into normal C57BL/6J. Leukemic BM was harvested and expanded ex vivo in RPMI-1640 media supplemented with 10% FBS, 1% P/S, recombinant IL-3 (10 ng/ml) and SCF (100 ng/ml).

Normal LKS/Lin⁻ BM cells were cultured in StemSpan™ SFEM II (Stemcell Technologies) with 1% P/S, recombinant FLT3-L (10 ng/ml), SCF (100 ng/ml) and TPO (10 ng/ml). As for primary human AML samples from patients, they were cultured in StemSpan™ H3000 (Stemcell Technologies) supplemented with 1% P/S and StemSpan™ CC100 (Stemcell Technologies).

Mice Housing

All mice were purchased from The Jackson Laboratory. Mice were maintained in pathogen-free conditions. Experiments involving mice were approved by the Massachusetts General Hospital (MGH) Institutional Animal Care and Use Committee (IACUC).

Virus Preparation and Spinfection

HEK293FT cells were co-transfected with packaging plasmids and viral vectors (Lentivirus: Delta 8.9, VSV-G and lentiviral plasmids; Retrovirus: Ampho/Eco vector and retroviral plasmids) using Lipofectamine 2000 (Thermo Fisher Scientific), Virus containing medium was collected after 48 hours and filtered using a 0.45 μm filter.

Human plasma fibronectin (EMD Millipore) was coated on tissue culture plates (4 μg/cm²) for 30 minutes at 37° C. Virus medium was mixed with the cells supplemented with 4 μg/ml Polybrene (Sigma-Aldrich) and subjected to spinfection (1000 g for 1 hour). Thereafter, the cells were washed and fresh complete medium was added.

Inducible MCT4 Knockdown/Knockout Systems

In this study, 2 inducible systems to suppress MCT4 expression in mouse AML were adopted. The first one is an inducible CRISPR-Cas9 system under the regulation of CreERT2. B6J.129(B6N)-Gt(ROSA)26Sor^{tm1(CAG-cas9*,-EGFP)Fezh}/J (Jackson Laboratory #026175) was crossed with B6.Cg-Ndor1^{Tg(UBC-cre/ERT2)1Ejb}/1J (Jackson Laboratory #007001). The GMP from the crossed offspring was infected with MLL-AF9 retrovirus and transformed into AML. Established AML cell lines were then infected with gRNA virus using lentiGuide-Puro plasmids, which was a gift from Feng Zhang (Addgene #52963). The gRNA sequences were obtained from mouse GeCKOv2 CRISPR knockout pooled library (gift from Feng Zhang Addgene #1000000052, #1000000053). The sequences of gRNA were summarized in Supplemental Table 3. The infected cell was selected by 10 ug/ml puromycin for 6 days in complete media. Cre expression was induced by 4-hydroxytamoxifen (1 μg/ml) (Sigma-Aldrich) in vitro. Successful recombination was confirmed by the presence of GFP⁺ cells.

The second method is an inducible shRNA system activated by tetracycline/doxycycline. Either scrambled RNA (AllStars Negative Control, Qiagen) or MCT4-targeting shRNA (Supplemental Table 3) were cloned into lentiviral Tet-pLKO-puro plasmid, which was a gift from Dmitri Wiederschain (Addgene #21915). The lentivirus was produced and MLL-AF9 AML cells were infected as aforementioned. Infected cells were selected by puromycin (10 μg/ml) for 6 days and cultured in RPMI-1640 media with 10% tetracycline-free FBS (Clontech), 1% P/S, recombinant IL-3 (10 ng/ml) and SCF (100 ng/ml). shRNA was induced by adding doxycycline (2 μg/ml) into culture media in vitro. As for in vivo induction, 625 mg doxycycline hyclate per kg diet (Envigo) was fed to the mice engrafted with shRNA infected MLL-AF9 AML cells at week-3 post transplantation for 12 days.

In human AML cell lines and primary patient myeloblasts, MCT4 was knocked down by shRNA (Supplemental Table 3). The shRNA was cloned into lentiviral pLKO.1 puro, a gift from Robert Weinberg (Addgene #8453). Infected cells were selected in puromycin (5 μg/ml) for 2 days before further experimentation.

Xenotransplantation and Engraftment Examination

Normal mouse HSPC was transplanted into mice for in vivo competitive assays. For mouse cell transplantation, either C57BL/6J or B6.SJL (purchased from Jackson Laboratory #000664, #002014) as the host, were lethally irradiated (2×6 Gy) before transplantation intravenously. Competitive carrier cells of WBM (200K) from the same species of host were included according to specific experimental designs. PB was drawn retro-orbitally every 4 weeks until 16-week post transplantation. After 16 weeks, the mouse BM was harvested. Different HSPC sub-population was examined by FACS using different fluorochrome-conjugated antibodies. For the secondary transplantation experiment, 1 million whole BM cells from the primary recipients were injected intravenously into irradiated mice. PB was drawn every 4 weeks and the secondary recipients were harvested at 16-week post transplantation and different HSPC subpopulation were analyzed by FACS.

For AML transplantation experiments, 100K-2M human AML or 500K-2M mouse MLL-AF9 AML cells were injected intravenously into sub-lethally irradiated (2.5 Gy) NSG and (4.5 Gy) B6.SJL mice respectively. BM aspiration of the femur was performed at various time points according to different experimental designs. The BM aspirated cells were assayed by FACS and the level of leukemic engraftment was determined. For human AML experiment, the percentage of human AML engraftment was calculated as human CD45⁺ cells/sum of human and mouse CD45⁺ cells in BM.

Measurement of Intracellular pH

Intracellular pH was measured by a fluorescent dye, SNARF-1 (Thermo Fisher Scientific) as reported previously (Man et al., 2014). In brief, PBS washed cells were incubated with 2.5 μM SNARF-1 at 37° C. for 20 minutes and then washed with PBS twice. Calibration of pHi was achieved by an ionophore nigericin (10 μM) in 100 mM K⁺ buffer with different standard pH. The pH-dependent shifts in emission spectra exhibited by SNARF-1 were used to calibrate the pHi among samples, as determined by the ratio of fluorescence intensities measured at 580 nm and 640 nm using BD FACSARIA III. In the presence of nigericin, the pHi of the cells was equalized to that of the K⁺ buffer. According to the SNARF-1 emission spectra in different standard solutions, a standard curve between pH and emission ratio was obtained. The cellular pHi was determined from the standard curve.

Analysis of Intracellular Metabolites

Cells were cultured in the presence of either unlabeled or ¹³C-labeled substrates such as [1,2-¹³C₂]glucose and [U-¹³C₆]glucose (Cambridge Isotope Laboratories) depending on the experimental design. After 24 hours, cell was harvested into micro-centrifuge tube and short spinned at full speed for 10 s. Supernatant was removed and washed with 0.9% NaCl solution. NaCl solution was removed after short spin. Methanol with internal standard (GC-MS: Norvaline; LC-MS: ¹³C-labelles bacterial extract) was added to quench and extract the cell pellet.

For GC-MS, the protocol was performed as described previously (Dong et al., 2017). In brief, the polar phase (glycolytic intermediates, TCA cycle metabolites and amino acids) and non-polar phase (fatty acids) were separated by two-phase extraction using methanol and chloroform. Each phase was then dried and the polar metabolites were derivatized by (methoxyamine) MOX and N-(tert-butyldimethylsilyl)-N-methyl-trifluoroacetamide (TBDMS). Metabolite abundance and mass isotopomer distribution (MID) were determined by GC-MS (Agilent GC 6890N and MSD 5975B) set at electron ionization (EI) mode. The injection volume per samples was 1 μL and the helium (carrier gas) flow rate was at 1 mL/min. The MSD source and quadrature temperatures were at 230° C. and 150° C., respectively. The inlet temperature of the GC column was at 270° C. The scan mode was used to detect the mass fragments ionized with an energy of 70 eV. After the completion of data collection, an in-house software coded within MATLAB was used to correct the natural abundance and numerically integrate the peaks in the chromatogram. The processed data were then organized in EXCEL and further analyzed to yield normalized metabolite abundance (by norvaline) and MID for metabolites of interest.

As for LC-MS, the samples were dried under nitrogen and subsequently resuspended in HPLC-grade water for LC-MS analysis. LC-MS was run in the Metabolite Profiling Core Facility at Whitehead Institute. A Dionex UltiMate 3000 UPLC system (Thermo Fisher Scientific) with a ZIC-pHILIC (5 μm polymer particle) 150×2.1 mm column (EMD Millipore) coupled to a QExactive orbitrap mass spectrometer (Thermo Fisher Scientific) was used for analysis. The mobile phase was run at a flow rate of 0.150 mL/min as a linear gradient from 80% B to 20% B between 0 and 20 mins, a linear gradient from 20% B to 80% B between 20 and 20.5 mins, and 80% B held from 20.5 to 28 mins, where solvent A was 20 mM ammonium carbonate+0.1% ammonium hydroxide and solvent B was acetonitrile. Column and autosampler temperatures were held at 25° C. and 4° C., respectively. Metabolites were ionized through electrospray ionization in the mass spectrometer, which operated in polarity switching mode scanning a range of 70-1,000 m/z. With retention times determined by authenticated the standards, resulting mass spectra and chromatograms were identified and processed using MAVEN software (Clasquin et al., 2012).

For preparation of the internal standards, Saccharomyces cerevisiae cells were grown on synthetic complete (SC) media except that natural abundance glucose was replaced with [U-¹³C₆]glucose (20 g/L [U-¹³C₆]glucose, 1.7 g/L yeast nitrogen base, and 5 g/L ammonium sulfate). After cultivation at 30° C. until OD600 reached ˜1, the intracellular metabolites were extracted and prepared following a previously reported protocol (Park et al., 2016). An aliquot of the internal standard was analyzed on LC-MS and uniform labeling with ¹³C was confirmed.

High Performance Liquid Chromatography

Culture media was collected at different time points with cells being filtered using 0.22 μm filter. 10 μL of the media sample was injected into an Agilent 1200 High-Performance Liquid Chromatography (HPLC) system for quantification of glucose and lactate. A Bio-Rad HPX-87H column coupled to a G1362 Refractive Index Detector were used and the mobile phase was 14 mM sulfuric acid with a flow rate of 0.7 mL/min. Standard curves were prepared using authenticated glucose and lactate standards purchased from Sigma-Aldrich.

Library Preparation, Nextseq RNA Sequencing and Data Analysis

Total RNA was extracted and purified by RNeasy Plus Mini Kit (Qiagen). Quality of RNA was determined using Agilent RNA 6000 Nano Kit (Agilent) and only RNA with RNA integrity (RIN)≥7.0 was used further. mRNA was isolated from 500 ng total RNA using NEBNext® Poly(A) mRNA Magnetic Isolation Module (New England Biolabs). DNA library was generated from the isolated mRNA using NEBNext®Ultra™II DNA Library Prep Kit for Illumina® (New England Biolabs). Adaptor and specific primer set were added to each DNA sample using NEBNext® Multiplex Oligo for Illumina® (Index Primers Set 1 & 2) (New England Biolabs).

Quality of the DNA library was examined (Tapestation 2200 and Kapa qPCR) in The Bauer Core Facility at Harvard University. All samples were combined and sequenced by the Bauer Core Facility at Harvard University using Illumina Nextseq High Yield 1×75 bp kit. 439293612 reads passed filtering, and each sample was sequenced at an average depth of 27455851. Those reads were mapped to the mouse genome (GRCm38-vM17) by HISAT2 (Kim et al., 2015). Expression of transcripts was quantified by RSEM (Martinez-Nunez and Sanford, 2016). All the subsequent analyses were performed using R. DESeq2 was used to process the raw counts of transcripts for normalization and dispersion estimation (Love et al., 2014). DESeq2's Negative Binomial GLM fitting and Wald tests were used to call differentially expressed genes with multiple test correction (adjusted p values less than 0.05). The R package clusterProfiler was used for GO over-representation analysis on significantly differentially expressed genes. The Gene Set Enrichment Analysis (GSEA) was also conducted using the R package clusterProfiler with Broad's hallmark gene sets from MSigDB.

Western Immunoblot

Cells were lyzed in 1×RIPA lysis buffer (Cell Signaling) with protease and phosphatase inhibitors. Cell lysate was centrifuged at 13000 rpm for 10 mins. Supernatant was collected and denatured in SDS-loading buffer with boiling for 10 mins. Cell lysates were separated, transferred and blotted with primary and horseradish peroxidase (HRP)-conjugated secondary antibodies (Supplemental Table 4). Hybridization signals were visualized with Amersham ECL Western blot detection reagents (GE Healthcare Amersham) or Luminata Forte Western HRP substrate (EMD Millipore) and captured by Hyperfilm™ ECL (GE Healthcare Amersham). Densitometric analysis of the bands was performed by ImageJ 1.8.0v.

Immunoprecipitation, In Vitro Enzymatic Assay and Binding Assay

Plasmids expressing different metabolic enzymes conjugated to different protein tags were purchased from Sino-biological Lab. Plasmids were expanded in TOP10 competent E. coli (Thermo Fisher Scientific) and purified by midi-prep kit (Macherey-Nagel) according to the manufacturer's protocol. Purified plasmids were transfected and overexpressed in HEK293FT cells by Lipofectamine 2000. After 48 hours, cells were harvested and lyzed by NP-40 based lysis buffer with protease and phosphatase inhibitors. Total protein (500 μg) in 1 ml lysate was incubated with agarose-conjugated anti-FLAG antibody (Thermo Fisher Scientific) at 4° C. overnight. The agarose beads were washed twice with lysis buffer. Purified agarose-protein conjugate was then subjected to in vitro enzymatic assay.

Different enzymes were subjected to different reaction conditions as reported (Kirkman and Gaetani, 1986; Lin et al., 2015; TeSlaa and Teitell, 2014). For each enzyme, the pH of reaction buffers was tittered ranged from 7.0 to 7.8. The rate of reaction was then detected by Synergy HTX multi-mode reader (Biotek).

As for the assay examining self-binding of the enzymes, same molecular ratio of HA-/FLAG-tag protein was transfected into HEK293FT cell using the same protocol aforementioned. After immunoprecipitating the FLAG-tag protein, the agarose-antibody-protein mixture was washed by washing buffers with various pH (7.0-7.9) with shaking at room temperature for 30 minutes twice. The protein was eluted using SDS-loading buffer with boiling for 10 minutes. The eluted protein was further subjected to Western immunoblot.

Luciferase Promoter Activity Assay

Three promoter regions of MCT4 were amplified from mouse gDNA using ExTaq DNA polymerase (Takara) (Supplemental Table 3). The DNA fragments were cloned into pGL4 luciferase reporter vector (Promega). pRL Renilla luciferase reporter vector (Promega) was co-transfected with pGL4 for normalizing the transfection efficiency in HEK293T. Two days after transfection, the cells were washed with PBS and subjected to dual-luciferase reporter assay system (Promega) according to the manufacturers' protocol. The luciferase signal was detected using Synergy HTX multi-mode reader.

Chromatin-Immunoprecipitation-PCR

ChIP assays were performed in hematopoietic cells as previously described (Kim et al., 2008) using Pierce™ Protein G Agarose (Thermo Fisher Scientific). In brief, the cell suspension was fixed with 1% formaldehyde for 7 mins then neutralized by glycine. The fixed cell was washed with cold PBS. The cell pellet was then sonicated in SDS ChIP buffer. The supernatant was collected and pre-cleared with protein G agarose. After pre-clearing, a ChIP reaction using different antibodies (1:100, summarized in Supplemental Table 4) was added to the lysate and incubated at 4° C. overnight Immunoprecipitated complexes were successively washed with buffers and then eluted using SDS elution buffer at 65° C. overnight to reverse crosslink protein-DNA complex. The samples were treated with RNase A and Proteinase K, then extracted by phenol-chloroform isoamyl alcohol and precipitated. Finally, the pellet was resuspended in 20 μl TE buffer.

The DNA was then subjected to Q-PCR analysis using specific primers (Supplemental Table 3) and SYBR™ Green PCR Master Mix (Thermo Fisher Scientific). Input genomic DNA was used for the reference sample. The reaction was run and analyzed by StepOnePlus Real-Time PCR System (Thermo Fisher Scientific). Mouse or human Gfi1b was included as a negative control.

RNA Purification. Reverse Transcription and Quantitative Real-Time PCR

Cell was washed twice with PBS and subjected to RNA purification using RNeasy Plus Mini Kit (Qiagen) according to the manufacturers' protocol. Extracted total RNA was then subjected to reverse transcription using SuperScript IV VILO Master Mix (Thermo Fisher Scientific) according to the manufacturers' protocol. Synthesized cDNA was then used for the Q-PCR using gene-specific primers (Supplemental Table 3) and SYBR™ Green PCR Master Mix. The reaction was run and analyzed by StepOnePlus Real-Time PCR System.

Proliferation Assays

The proliferation rate was determined by BrdU incorporation assay both in vitro and in vivo using BD Pharmingen BrdU Flow Kit (BD) according to the manufacturers' protocol. To label cell in vitro, 1 mM BrdU dissolved in PBS was added to each mL of cell culture medium directly and the cell was harvested after 1 hour. To label cell in vivo, 50 mg of BrdU per kg animal was injected into mice intraperitoneally and the BM was harvested 1 hour after injection. The harvested cell was then fixed, permeabilized and stained as protocol suggested. 7-AAD was added to stain total DNA content and analyzed by FACS using BD FACSARIA III.

The proliferation rate was also determined by CellTrace™ Violet Cell Proliferation Kit (Thermo Fisher Scientific) according to the manufacturers' protocol. In brief, at day 2 post Cas9 induction in AML, cells were stained with the dye and cultured in either 20% or 2% O₂ conditions for 2 more days. The fluorescence intensity of CellTrace™ Violet was then examined by FACS.

Glucose Uptake Assay

Both in vitro and in vivo glucose uptake were assayed in this study. For in vitro assay, Glucose Uptake-Glo™ Assay (Promega) was used. The experiment was done according to the manufacturers' protocol. In brief, cells were incubated with 2DG. After incubation, cells were lyzed and detection reagent measuring 2DG6P was added. The signal was detected by Synergy HTX multi-mode reader (Biotek). For in vivo glucose uptake assay, 2-NBDG (2-(N-(7-nitrobenz-2-oxa-1,3-diazol-4-yl)amino)-2-deoxyglucose) (Cayman Chemical) was used. 5 mg/kg 2-NBDG diluted in PBS was injected intravenously. The mouse cells were harvested after 10 minutes. The level of glucose uptake was determined by the fluorescence intensity of 2NBDG inside the cells using FACS.

Glycolysis and Mito Stress Analysis

Glycolysis stress and Mito stress of normal Lin⁻ BM cells and AML were examined by Seahorse XF analyzer (Agilent) according to the manufacturers' protocol. In brief, the suspension cells were seeded onto a Cell-Tak (Corning) coated Seahorse XF Microplate in Seahorse XF Base Medium with appropriate supplement (Glycolysis Stress test: 1 mM glutamine; Mito stress test: 1 mM pyruvate, 2 mM glutamine and 10 mM glucose) and centrifuged at 200×g for 5 mins Different compounds were injected for the ECAR and OCR profiling (Glycolysis Stress test: 10 mM glucose, 1 μM Oligomycin and 50 mM 2DG; Mito Stress test: 1 μM Oligomycin, 0.5 μM FCCP and 0.5 μM Antimycin A). The data was eventually analyzed by Seahorse Wave Desktop 2.6 (Agilent).

In Vitro ROS Detection

For the investigation of cellular ROS level, two fluorescent dyes were used, CellROX™ Deep Red Reagent and MitoSOX™ Red Mitochondrial Superoxide Indicator (Thermo Fisher Scientific). Cells were stained according to the protocol suggested by the manufacturer. The fluorescent signal of CellROX™/MitoSOX™ were excited by 640 nm/488 nm and detected by 675 nm/575 nm using BD FACSARIA III.

Colony Forming Assay

Clonogenic activity of normal HSPC and AML cells was evaluated by methylcellulose-based culture (Mouse: MethoCult™ M3434; Human: MethoCult™ H4434, Stem Cell Technologies). Normal LKS and AML were seeded at 100 and 1000 cells/ml in triplicates respectively and colonies were examined after 10 days of culture.

Cytospins and Wright-Giemsa Staining

Cells were washed with and resuspended in 200 μl PBS at 2 million/ml. Cytospins (Thermo Scientific Shandon) were done at 1,000 rpm for 60 s and the cells were allowed to air dry. Cells were stained in 100% Wright-Giemsa (Siemens) for 2 min, and in 20% Wright-Giemsa diluted in buffer for 12 min. Stained cells were rinsed in deionized water, and coverslips were fixed using Permount prior to microscopic examination.

In Vivo Multi-Photon Fluorescence Imaging

The Olympus FVMPE-RS multiphoton imaging system was used for intravital imaging of the mouse with pH reporter. Detail to prepare the mouse for the intravital imaging is described as previous works (Lo Celso et al., 2011). In short, the calvarial BM of the mouse was accessed optically after a simple skin flap surgery and the underlying bone surface was exposed. Mouse restrainer was used to minimize the motion of the mouse during imaging. Two fluorescence detection channels were used to display SEpHluorin (green) and mCherry (red). Second harmonic generation (SHG) from the collagen fibers in the calvarial bone and two photon fluorescence from SEpHluorin were excited by 920 nm femtosecond laser (Mai-Tai HP DS-OL, Spectra Physics), and one photon fluorescence from mCherry was excited by 1095 nm femtosecond laser (Insight DS-OL, Spectra Physics). Two lasers (920 nm and 1095 nm) were excited to the BM at the same time so that all fluorescences could be acquired simultaneously. For collection of emitted fluorescence, the optical band pass filters with the wavelength range of 495-540 and 575-645 nm were used for SEpHluorin and mCherry respectively. Water immersion objective lens (XLPLN25XWM, 25×, 1.05NA, 2 mm walking distance, Olympus) was used for the multiphoton imaging.

Statistical Analysis

GraphPad PRISM 7 software was used to preform statistical analyses. Paired Student's t-test was used for analyzing pairwise comparison of experiments. Kaplan Meier survival analysis was used for the survival curves analyses. Mann-Whitney U test was used for the comparison for primary AML experiments. p-value smaller than 0.05 was considered as statistically significant. Data represented as the mean±SEM. (*: p<0.05; **: p<0.01; ***: p<0.001).

SUPPLEMENTAL TABLE 1
Clinical information of primary AML patient samples

| | | | | Genetic mutation* | | |
|---|---|---|---|---|---|---|
| | | % of | Disease | FLT3- | | DNMT |
| Patient | Cytogenetics | blast | stage | ITD | NPM1c | 3A^{R882} |
| AML1 | 48, XY, +8, | 76 | Diagnostic | 1 | 0 | 0 |
| | inv(16) | | | | | |
| | (p13.1q22), | | | | | |
| | +22[17] | | | | | |
| AML2 | 46, XX[23] | 66 | Diagnostic | 1 | 0 | 0 |
| AML3 | 49, XY, +12, | 68 | Diagnostic | 0 | 0 | 0 |
| | +21, +22[5]/ | | | | | |
| | 46, XY[3] | | | | | |
| AML4 | 46, XY, | 61 | Diagnostic | 0 | 0 | 1 |
| | add(7)(q11.2) | | | | | |
| | [17] | | | | | |
| AML5 | 46, XX, del(11) | 90.5 | Diagnostic | 0 | 1 | 0 |
| | (q23)[20] | | | | | |
| AML6 | 46, XX[17] | 88 | Diagnostic | 0 | 0 | 0 |
| AML7 | 47, XX, | 62 | Diagnostic | 0 | 0 | 1 |
| | inv(16) | | | | | |
| | (p13.1q22), | | | | | |
| | +22[9] | | | | | |
| AML8 | 46, XX[20] | 80 | Diagnostic | 0 | 1 | 0 |
| AML9 | 46, XX[13] | 92 | Diagnostic | 0 | 0 | 1 |
| AML10 | 45, XY, add(8) | 89 | Diagnostic | 0 | 0 | 0 |
| | (q24), add(16) | | | | | |
| | (q24), −17, | | | | | |
| | i(21)(q10), | | | | | |
| | i(22)(q10)[14]/ | | | | | |
| | 46, XY[1] | | | | | |
| AML11 | 46, XX, | 68 | Diagnostic | 0 | 0 | 0 |
| | t(7;11)(p15, | | | | | |
| | p15)[20] | | | | | |
| AML12 | 47, add(X) | 98 | Diagnostic | 0 | 0 | 0 |
| | (q22)(del(X) | | | | | |
| | (q22), +8[17] | | | | | |
| AML13 | 46, XX[13] | 96 | Diagnostic | 1 | NA | NA |
| AML14 | 46, XY[18] | 62 | Diagnostic | 1 | 1 | 0 |
| AML15 | poor growth | 80 | Diagnostic | 1 | NA | NA |
| AML16 | 46, XX[21] | 61 | Diagnostic | 0 | 0 | 0 |

*Genetic mutation:
1—mutated,
0—wild-type,
NA—not available

Supplemental TABLE 2
Genetics information of human leukemic cell lines

| Cell line | Cell type | Genetics |
|---|---|---|
| K562 | CML in blast crisis | BCR-ABL1 |
| KG1 | AML | FGFR10P2-FGFR1 |
| ML2 | AML | MLL-AF6 |
| Kasumi-1 | AML | AML1-ETO, c-Kit^{N822L} |
| MOLM-13 | AML | MLL-AF9, FLT3-ITD |
| MOLM-14 | AML | MLL-AF9, FLT3-ITD |
| MV4-11 | AML | MLL-AF4, FLT3-ITD |
| THP-1 | AML | MLL-AF9 |
| MONO-MAC6 | AML | MLL-AF9 |
| HL-60 | AML | MYC amplification |
| NB4 | APL | PML-RARA |
| U937 | Histocytic lymphoma | NA |
| NOMO-1 | AML | MLL-AF9 |

SUPPLEMENTAL TABLE 3
Summary of DNA oligo sequences
Q-PCR primers

| Gene | Forward primer | Reverse primer |
|---|---|---|
| Mouse | ATTCAGTGCAACGACC | CGGCTGCCGTATTT |
| MCT1 | AGTG(SEQ ID NO: 1) | ATTCAC (SEQ ID |
| | | NO: 2) |
| Mouse | CTTGTGGGTGGCCTCTT | TGGAAGTTGAGAGC |
| MCT4 | TG (SEQ ID NO: 3) | CAGACC(SEQ ID |
| | | NO: 4) |
| Mouse | TCTCCCTCTGGATTCTC | TACGATCAGCAGGC |
| NHE1 | CTG (SEQ ID NO: 5) | AGCTCT(SEQ ID |
| | | NO: 6) |
| Mouse | ACCTAACCATCCCTGTG | GAGGTACTGCTGGG |
| AE1 | ACC (SEQ ID NO: 7) | GACGTA(SEQ ID |
| | | NO: 8) |
| Mouse | CGCTAGACGGACGACA | GGAAGTCCTTGTGC |
| CA2 | ACTT(SEQ ID NO: 9) | CAGTTC (SEQ ID |
| | | NO: 10) |
| Mouse | TCTCCCTCTGGATTCTC | TACGATCAGCAGGC |
| CA9 | CTG (SEQ ID NO: 11) | AGCTCT(SEQ ID |
| | | NO: 12) |
| Mouse | CCTTAAGAAGCAGCCTT | CCTCCACACGATGG |
| CA12 | CCA (SEQ ID NO: 13) | GTACTT(SEQ ID |
| | | NO: 14) |
| Mouse | CCCACAGGGTCTGCTTA | ACGTCTACCACGAA |
| V-ATPase | CAA (SEQ ID NO: 15) | GCGTCT(SEQ ID |
| | | NO: 16) |
| Human | TGGCTGTCATGTATGGT | AGCTGCAATCAAGC |
| MCT4 | GGA (SEQ ID NO: 17) | CACAG(SEQ ID |
| | | NO: 18) |
| β-actin | AAATCTGGCACCACAC | GGGGTGTTGAAGGT |
| | CTTC(SEQ ID NO: 19) | CTCAAA (SEQ ID |
| | | NO: 20) |

| | |
|---|---|
| Gene | Sense sequence |

gRNA sequences

| | |
|---|---|
| Mouse MCT1 | 1. AGCCGTCCAGTAATGATCGC |
| | (SEQ ID NO: 21) |
| | 2. CTTCTCGTCGACATCGGTGC |
| | (SEQ ID NO: 22) |
| | 3. CCTTTGTCTACAACCTACGT |
| | (SEQ ID NO: 23) |
| | 4. TGTGTCTACGCCGGAGTCTT |
| | (SEQ ID NO: 24) |
| Mouse MCT4 | 1. AAGCGTCGCCCTATTGCCAA |
| | (SEQ ID NO: 25) |
| | 2. GTGCTCATCGGACCCCCGTC |
| | (SEQ ID NO: 26) |
| | 3. TTGGCTACAGCGACACGGCT |
| | (SEQ ID NO: 27) |
| | 4. GAAAAAGACGCTGACCGCCT |
| | (SEQ ID NO: 28) |
| Mouse NHE1 | 1. TCTCTCCGACGCCCTTGATC |
| | (SEQ ID NO: 29) |
| | 2. TCCGACTCACGCCATGATTC |
| | (SEQ ID NO: 30) |
| | 3. TTCTCCGTGAACTGCCGCAG |
| | (SEQ ID NO: 31) |
| | 4. AGGGGCCATCGCCTTCTCGC |
| | (SEQ ID NO: 32) |
| Mouse AE1 | 1. CCCATACACCATCCTCTCGA |
| | (SEQ ID NO: 33) |
| | 2. TCTAGACTGCTTCATCTACG |
| | (SEQ ID NO: 34) |
| | 3. GTCCTCACCTGACCGGAGCT |
| | (SEQ ID NO: 35) |
| | 4. AGCAGTTCTTCTCGGTCCTG |
| | (SEQ ID NO: 36) |
| Mouse CA2 | 1. GTCATCAAACTCAACGTTAA |
| | (SEQ ID NO: 37) |
| | 2. AAAGCTGTGCAGCAACCGGA |
| | (SEQ ID NO: 38) |
| | 3. CCCAAAACAGCCAATCCATC |
| | (SEQ ID NO: 39) |
| | 4. AGCCCCAGTGAAAGTGAAAC |
| | (SEQ ID NO: 40) |
| Mouse CA9 | 1. TTGCAGAGTGCGGCAGAATG |
| | (SEQ ID NO: 41) |
| | 2. GTCCCCGGTAGACATCCGCC |
| | (SEQ ID NO: 42) |
| | 3. CAGTACTGAGGTGCACCACG |
| | (SEQ ID NO: 43) |
| | 4. CCAGTGTAGATGCAACTGCA |
| | (SEQ ID NO: 44) |
| Mouse CA12 | 1. CTTACCAACATAGGTCCACT |
| | (SEQ ID NO: 45) |
| | 2. TCCAAGAAGTACCCATCGTG |
| | (SEQ ID NO: 46) |
| | 3. ACCGCCAGTGACAAGTCCGA |
| | (SEQ ID NO: 47) |
| | 4. GGGAACCGCAATGACCCCCA |
| | (SEQ ID NO: 48) |
| Mouse | 1. CGCCTTCCAGAGACGCTTCG |
| V-ATPase | (SEQ ID NO: 49) |
| | 2. CGTCTACCACGAAGCGTCTC |
| | (SEQ ID NO: 50) |
| | 3. ATGCGCAGCAGGTCTCGGGG |
| | (SEQ ID NO: 51) |
| | 4. CCGAGACCTGCTGCGCATCC |
| | (SEQ ID NO: 52) |
| Mouse LDHA | 1. CTGCTGATCGTCTCCAATCC |
| | (SEQ ID NO: 53) |
| | 2. TTTCCCAAAAACCGAGTAAT |
| | (SEQ ID NO: 54) |
| Control gRNA | 1. GCTTTCACGGAGGTTCGACG |
| | (SEQ ID NO: 55) |
| | 2. ATGTTGCAGTTCGGCTCGAT |
| | (SEQ ID NO: 56) |

shRNA sequences

| | |
|---|---|
| Mouse MCT4 | GCTGGATGCAACCAAAGTTTA |
| | (SEQ ID NO: 57) |
| | AGGAGCTTATGCATGAGTTTG |
| | (SEQ ID NO: 58) |
| Mouse MCT1 | GCAGTATCTTGGTGAATAAAT |
| | (SEQ ID NO: 59) |
| | CCAGTGAAGTATCATGGATAT |
| | (SEQ ID NO: 60) |
| Human MCT4 | TGCATTAGGAAGAAGCCCAAA |
| | (SEQ ID NO: 61) |
| | GCTCATACAGGAGTTTGGGAT |
| | (SEQ ID NO: 62) |
| MLL-AF9 | TTCTTTTCAGACTTGTTGG |
| | (SEQ ID NO: 63) |
| | GTTTTCTTTTCAGACTTGT |
| | (SEQ ID NO: 64) |
| AllStars | GGAATCTCATTCGATGCATAC |
| Control | (SEQ ID NO: 65) |

| | |
|---|---|
| Forward primer | Reverse primer |

Primers for amplifying mouse
MCT4 promoter region

| | | |
|---|---|---|
| Promoter | | |
| region | | |
| Seql | AGCTGCTGTCCTGTCCTCAT | TCTCTCCACAAAT |
| | (SEQ ID NO: 66) | GGTGTGC(SEQ |
| | | ID NO: 67) |
| Seq2 | CATGGTTCCTAGGGTCAGGA | ATGAGGACAGGAC |
| | (SEQ ID NO: 68) | AGCAGCT (SEQ |
| | | ID NO: 69) |
| Seq3 | TGGGTGCTGGAAATCTAACC | TCCTGACCCTAGG |
| | (SEQ ID NO: 70) | AACCATG (SEQ |
| | | ID NO: 71) |

ChIP-PCR primers

| | | |
|---|---|---|
| Gene | | |
| Mouse | GGAATGCTACAGCCTCCTTG | AAAGAGACCCGAG |
| MCT4 | (SEQ ID NO: 72) | GGCATAC (SEQ |
| | | ID NO: 73) |
| Mouse | GATACAGAGCGGTTCATACA | TCGCCAGTCAACA |
| HoxA9 | G (SEQ ID NO: 74) | TCAAGAG (SEQ |
| | | ID NO: 75) |
| Mouse | CGCCAGATTTTGACACAAAT | CTGCACAGACAGA |
| Gfi1b | AA (SEQ ID NO: 76) | CACTTCTCC |
| | | (SEQ ID NO: |
| | | 77) |
| Human | CTGCCTCCTTTGTGTGTGAA | GGCCACAGGAATG |
| MCT4 | (SEQ ID NO: 78) | CTTTAAC (SEQ |
| | | ID NO: 79) |
| Human | ATAGTCTGCATGGGGTCCAG | TGCAGATTGGTGG |
| Gfi1b | (SEQ ID NO: 80) | AACTGAG (SEQ |
| | | ID NO: 81) |

What is claimed is:

1. A method of treating leukemia in a subject in need thereof, comprising administering to the subject a therapeutically effective amount of an agent that inhibits the activity or expression of a proton exporter.

2. The method of claim 1, wherein the proton exporter is Monocarboxylate Transporter 4 (MCT4) or Sodium-hydrogen antiporter 1 (NHE1).

3. The method of claim 1, wherein the agent does not inhibit Monocarboxylate Transporter 1 (MCT1) activity or expression.

4. The method of claim 1, wherein the agent inhibits the growth, viability or clonogenic ability of leukemia initiating cells (LICs).

5. The method of claim 1, wherein the leukemia exhibits increased intracellular pH (pHi) as compared to non-leukemic blood cells.

6. The method of claim 1, wherein the leukemic cells exhibit increased MCT4 expression or activity or increased NHE1 expression or activity.

7. The method of claim 1, wherein the agent does not inhibit the growth, viability, or clonogenic ability of non-leukemic blood cells.

8. The method of claim 1, wherein the subject is administered a second anti-leukemic agent selected from a glycolysis inhibitor, a histone deacetylase inhibitor, or a pentose phosphate pathway (PPP) inhibitor.

9. A method of inhibiting the growth, viability, or clonogenic ability of a cancer cell, comprising contacting the cancer cell with an agent that decreases the intracellular pH (pHi) of the cancer cell.

10. The method of claim 9, wherein the cancer cell exhibits increased intracellular pH (pHi) as compared to a non-cancer cell.

11. The method of claim 9, wherein the cancer cell exhibits increased activity or expression of a proton exporter selected from Monocarboxylate Transporter 4 (MCT4) and Sodium-hydrogen antiporter 1 (NHE1) as compared to a non-cancer cell.

12. The method of claim 9, wherein the agent does not inhibit MCT1 activity or expression.

13. The method of claim 9, wherein the agent preferentially inhibits the growth, viability, or clonogenic ability of a cancer cell in a low oxygen environment.

14. The method of claim 9, wherein the agent does not inhibit the growth or viability of non-cancerous cells.

15. The method of claim 9, wherein the cancer is leukemia.

16. The method of claim 9, wherein the agent is administered to a subject having cancer.

17. The method of claim 16, wherein a second anti-cancer agent is administered to the subject.

18. A method of preventing, delaying, reducing the likelihood of relapse of, or reducing the likelihood of leukemia in a subject in need thereof, comprising administering to the patient a therapeutically effective amount of a Monocarboxylate Transporter 4 (MCT4) inhibitor or a Sodium-hydrogen antiporter 1 (NHE1) inhibitor.

19. The method of claim 18, wherein the subject has one or more risk factors associated with the development of leukemia.

20. A method of increasing the growth or proliferation of a cell comprising contacting the cell with an agent that increases the expression or activity of a proton exporter.


SEQUENCE LISTING (81 sequences)

SEQ ID NO: 1 - DNA, 20 bp, Artificial Sequence
  Synthetic construct
attcagtgcaacgaccagtg

SEQ ID NO: 2 - DNA, 20 bp, Artificial Sequence
  Synthetic construct
cggctgccgtatttattcac

SEQ ID NO: 3 - DNA, 19 bp, Artificial Sequence
  Synthetic construct
cttgtgggtggcctctttg

SEQ ID NO: 4 - DNA, 20 bp, Artificial Sequence
  Synthetic construct
tggaagttgagagccagacc

SEQ ID NO: 5 - DNA, 20 bp, Artificial Sequence
  Synthetic construct
tctccctctggattctcctg

SEQ ID NO: 6 - DNA, 20 bp, Artificial Sequence
  Synthetic construct
tacgatcagcaggcagctct

SEQ ID NO: 7 - DNA, 20 bp, Artificial Sequence
  Synthetic construct
acctaaccatccctgtgacc

SEQ ID NO: 8 - DNA, 20 bp, Artificial Sequence
  Synthetic construct
gaggtactgctggggacgta

SEQ ID NO: 9 - DNA, 20 bp, Artificial Sequence
  Synthetic construct
cgctagacggacgacaactt

SEQ ID NO: 10 - DNA, 20 bp, Artificial Sequence
  Synthetic construct
ggaagtccttgtgccagttc

SEQ ID NO: 11 - DNA, 20 bp, Artificial Sequence
  Synthetic construct
tctccctctggattctcctg

SEQ ID NO: 12 - DNA, 20 bp, Artificial Sequence
  Synthetic construct
tacgatcagcaggcagctct

SEQ ID NO: 13 - DNA, 20 bp, Artificial Sequence
  Synthetic construct
ccttaagaagcagccttcca

SEQ ID NO: 14 - DNA, 20 bp, Artificial Sequence
  Synthetic construct
cctccacacgatgggtactt

SEQ ID NO: 15 - DNA, 20 bp, Artificial Sequence
  Synthetic construct
cccacagggtctgcttacaa

SEQ ID NO: 16 - DNA, 20 bp, Artificial Sequence
  Synthetic construct
acgtctaccacgaagcgtct

SEQ ID NO: 17 - DNA, 20 bp, Artificial Sequence
  Synthetic construct
tggctgtcatgtatggtgga

SEQ ID NO: 18 - DNA, 19 bp, Artificial Sequence
  Synthetic construct
agctgcaatcaagccacag

SEQ ID NO: 19 - DNA, 20 bp, Artificial Sequence
  Synthetic construct
aaatctggcaccacaccttc

SEQ ID NO: 20 - DNA, 20 bp, Artificial Sequence
  Synthetic construct
ggggtgttgaaggtctcaaa

SEQ ID NO: 21 - DNA, 20 bp, Artificial Sequence
  Synthetic construct
agccgtccagtaatgatcgc

SEQ ID NO: 22 - DNA, 20 bp, Artificial Sequence
  Synthetic construct
cttctcgtcgacatcggtgc

SEQ ID NO: 23 - DNA, 20 bp, Artificial Sequence
  Synthetic construct
cctttgtctacaacctacgt

SEQ ID NO: 24 - DNA, 20 bp, Artificial Sequence
  Synthetic construct
tgtgtctacgccggagtctt

SEQ ID NO: 25 - DNA, 20 bp, Artificial Sequence
  Synthetic construct
aagcgtcgccctattgccaa

SEQ ID NO: 26 - DNA, 20 bp, Artificial Sequence
  Synthetic construct
gtgctcatcggacccccgtc

SEQ ID NO: 27 - DNA, 20 bp, Artificial Sequence
  Synthetic construct
ttggctacagcgacacggct

SEQ ID NO: 28 - DNA, 20 bp, Artificial Sequence
  Synthetic construct
gaaaaagacgctgaccgcct

SEQ ID NO: 29 - DNA, 20 bp, Artificial Sequence
  Synthetic construct
tctctccgacgcccttgatc

SEQ ID NO: 30 - DNA, 20 bp, Artificial Sequence
  Synthetic construct
tccgactcacgccatgattc

SEQ ID NO: 31 - DNA, 20 bp, Artificial Sequence
  Synthetic construct
ttctccgtgaactgccgcag

SEQ ID NO: 32 - DNA, 20 bp, Artificial Sequence
  Synthetic construct
aggggccatcgccttctcgc

SEQ ID NO: 33 - DNA, 20 bp, Artificial Sequence
  Synthetic construct
cccatacaccatcctctcga

SEQ ID NO: 34 - DNA, 20 bp, Artificial Sequence
  Synthetic construct
tctagactgcttcatctacg

SEQ ID NO: 35 - DNA, 20 bp, Artificial Sequence
  Synthetic construct
gtcctcacctgaccggagct

SEQ ID NO: 36 - DNA, 20 bp, Artificial Sequence
  Synthetic construct
agcagttcttctcggtcctg

SEQ ID NO: 37 - DNA, 20 bp, Artificial Sequence
  Synthetic construct
gtcatcaaactcaacgttaa

SEQ ID NO: 38 - DNA, 20 bp, Artificial Sequence
  Synthetic construct
aaagctgtgcagcaaccgga

SEQ ID NO: 39 - DNA, 20 bp, Artificial Sequence
  Synthetic construct
cccaaaacagccaatccatc

SEQ ID NO: 40 - DNA, 20 bp, Artificial Sequence
  Synthetic construct
agccccagtgaaagtgaaac

SEQ ID NO: 41 - DNA, 20 bp, Artificial Sequence
  Synthetic construct
ttgcagagtgcggcagaatg

SEQ ID NO: 42 - DNA, 20 bp, Artificial Sequence
  Synthetic construct
gtccccggtagacatccgcc

SEQ ID NO: 43 - DNA, 20 bp, Artificial Sequence
  Synthetic construct
cagtactgaggtgcaccacg

SEQ ID NO: 44 - DNA, 20 bp, Artificial Sequence
  Synthetic construct
ccagtgtagatgcaactgca

SEQ ID NO: 45 - DNA, 20 bp, Artificial Sequence
  Synthetic construct
cttaccaacataggtccact

SEQ ID NO: 46 - DNA, 20 bp, Artificial Sequence
  Synthetic construct
tccaagaagtacccatcgtg

SEQ ID NO: 47 - DNA, 20 bp, Artificial Sequence
  Synthetic construct
accgccagtgacaagtccga

SEQ ID NO: 48 - DNA, 20 bp, Artificial Sequence
  Synthetic construct
gggaaccgcaatgaccccca

SEQ ID NO: 49 - DNA, 20 bp, Artificial Sequence
  Synthetic construct
cgccttccagagacgcttcg

SEQ ID NO: 50 - DNA, 20 bp, Artificial Sequence
  Synthetic construct
cgtctaccacgaagcgtctc

SEQ ID NO: 51 - DNA, 20 bp, Artificial Sequence
  Synthetic construct
atgcgcagcaggtctcgggg

SEQ ID NO: 52 - DNA, 20 bp, Artificial Sequence
  Synthetic construct
ccgagacctgctgcgcatcc

SEQ ID NO: 53 - DNA, 20 bp, Artificial Sequence
  Synthetic construct
ctgctgatcgtctccaatcc

SEQ ID NO: 54 - DNA, 20 bp, Artificial Sequence
  Synthetic construct
tttcccaaaaaccgagtaat

SEQ ID NO: 55 - DNA, 20 bp, Artificial Sequence
  Synthetic construct
gctttcacggaggttcgacg

SEQ ID NO: 56 - DNA, 20 bp, Artificial Sequence
  Synthetic construct
atgttgcagttcggctcgat

SEQ ID NO: 57 - DNA, 21 bp, Artificial Sequence
  Synthetic construct
gctggatgcaaccaaagttta

SEQ ID NO: 58 - DNA, 21 bp, Artificial Sequence
  Synthetic construct
aggagcttatgcatgagtttg

SEQ ID NO: 59 - DNA, 21 bp, Artificial Sequence
  Synthetic construct
gcagtatcttggtgaataaat

SEQ ID NO: 60 - DNA, 21 bp, Artificial Sequence
  Synthetic construct
ccagtgaagtatcatggatat

SEQ ID NO: 61 - DNA, 21 bp, Artificial Sequence
  Synthetic construct
tgcattaggaagaagcccaaa

SEQ ID NO: 62 - DNA, 21 bp, Artificial Sequence
  Synthetic construct
gctcatacaggagtttgggat

SEQ ID NO: 63 - DNA, 19 bp, Artificial Sequence
  Synthetic construct
ttcttttcagacttgttgg

SEQ ID NO: 64 - DNA, 19 bp, Artificial Sequence
  Synthetic construct
gttttcttttcagacttgt

SEQ ID NO: 65 - DNA, 21 bp, Artificial Sequence
  Synthetic construct
ggaatctcattcgatgcatac

SEQ ID NO: 66 - DNA, 20 bp, Artificial Sequence
  Synthetic construct
agctgctgtcctgtcctcat

SEQ ID NO: 67 - DNA, 20 bp, Artificial Sequence
  Synthetic construct
tctctccacaaatggtgtgc

SEQ ID NO: 68 - DNA, 20 bp, Artificial Sequence
  Synthetic construct
catggttcctagggtcagga

SEQ ID NO: 69 - DNA, 20 bp, Artificial Sequence
  Synthetic construct
atgaggacaggacagcagct

SEQ ID NO: 70 - DNA, 20 bp, Artificial Sequence
  Synthetic construct
tgggtgctggaaatctaacc

SEQ ID NO: 71 - DNA, 20 bp, Artificial Sequence
  Synthetic construct
tcctgaccctaggaaccatg

SEQ ID NO: 72 - DNA, 20 bp, Artificial Sequence
  Synthetic construct
ggaatgctacagcctccttg

SEQ ID NO: 73 - DNA, 20 bp, Artificial Sequence
  Synthetic construct
aaagagacccgagggcatac

SEQ ID NO: 74 - DNA, 21 bp, Artificial Sequence
  Synthetic construct
gatacagagcggttcatacag

SEQ ID NO: 75 - DNA, 20 bp, Artificial Sequence
  Synthetic construct
tcgccagtcaacatcaagag

SEQ ID NO: 76 - DNA, 22 bp, Artificial Sequence
  Synthetic construct
cgccagattttgacacaaataa

SEQ ID NO: 77 - DNA, 22 bp, Artificial Sequence
  Synthetic construct
ctgcacagacagacacttctcc

SEQ ID NO: 78 - DNA, 20 bp, Artificial Sequence
  Synthetic construct
ctgcctcctttgtgtgtgaa

SEQ ID NO: 79 - DNA, 20 bp, Artificial Sequence
  Synthetic construct
ggccacaggaatgctttaac

SEQ ID NO: 80 - DNA, 20 bp, Artificial Sequence
  Synthetic construct
atagtctgcatggggtccag

SEQ ID NO: 81 - DNA, 20 bp, Artificial Sequence
  Synthetic construct
tgcagattggtggaactgag